(19) United States
(12) Patent Application Publication
(10) Pub. No.: US 2024/0124941 A1
(43) Pub. Date: Apr. 18, 2024

(54) MULTI-MODAL METHODS AND SYSTEMS OF DISEASE DIAGNOSIS

(71) Applicant: Micronoma, Inc., San Diego, CA (US)

(72) Inventors: Gregory D. POORE, San Diego, CA (US); Serena FRARACCIO, San Diego, CA (US); Stephen WANDRO, San Diego, CA (US); Akanksha SINGH-TAYLOR, San Diego, CA (US); Cameron MARTINO, San Diego, CA (US); Eddie ADAMS, San Diego, CA (US); Sandrine MILLER-MONTGOMERY, San Diego, CA (US)

(21) Appl. No.: 18/490,595

(22) Filed: Oct. 19, 2023

Related U.S. Application Data
(63) Continuation of application No. PCT/US2023/075642, filed on Sep. 29, 2023.
(60) Provisional application No. 63/412,369, filed on Sep. 30, 2022.

(51) Int. Cl. C12Q 1/6886; G16H 50/20; G16H 50/70

(52) U.S. Cl. CPC C12Q 1/6886; G16H 50/20; G16H 50/70

(57) ABSTRACT

Provided herein are multi-modal methods and/or systems of diagnosing one or more disease, as described elsewhere herein.

### CROSS-REFERENCE

This application claims the benefit of PCT/US2023/075642 filed on Sep. 29, 2023, which claims priority to U.S. Provisional Patent Application No. 63/412,369 filed Sep. 30, 2022, which applications are incorporated herein by reference in their entirety.

### BACKGROUND

Cancer was widely considered a sterile tissue until recently, and methods to diagnose it have thus relied on the detection of low-to-high plex human biomarkers. However, since tumor size limits its molecular abundances in circulation, such as circulating tumor DNA (ctDNA), these methods' sensitivities for detecting early-stage disease has been challenging. Targeting more biomarkers, sequencing deeper, and/or testing more frequently have been employed to improve early-stage detection, but they do not circumvent biologically imposed constraints that can impede clinical utility.

Prior art in a related field may include: US2018/0223338, US2018/0258495, WO 2019/191649, WO 2019/079635, WO 2022/140386, or WO 2022/212283.

### SUMMARY

Lung cancer is exemplary of this difficulty, as the leading cause of cancer-related deaths worldwide, with ˜23% of patients diagnosed at localized stage in the United States. Although lung cancer screening through low-dose computed tomography (LDCT) improves early-stage diagnosis and reduces mortality in at-risk individuals, low patient adherence has restricted its benefits. Minimally invasive liquid biopsies could improve screening compliance but have lower sensitivity in early-stage disease than LDCT, which reliably detects nodules as small as 4 millimeters in diameter. For example, in validation cohorts comparing stage I disease to predominantly healthy controls, a commercially-available cell-free DNA (cfDNA) methylation assay reported ˜25% sensitivity at 99.3% specificity (PMID: 33506766); a ctDNA-based assay reported ˜30% average sensitivity at 98% specificity (PMID: 32269342); a fragmentomic assay reported ˜50% sensitivity at 80% specificity (PMID: 34417454); and an integrated multi-omic test reported ˜40% sensitivity at 96.3% specificity (PMID: 29348365). Conversely, LDCT sensitivity is between 59-100%. These discrepancies underscore the need to approach early-stage lung cancer detection differently.

Another diagnostic challenge stems from difficult management of indeterminate pulmonary nodules (IPNs), which are non-calcified masses 6-30 millimeters in diameter with equivocal malignancy statuses. Incidental pulmonary nodules are reported on ˜30% of chest CTs, with approximately >1.5 million nodules detected annually in the United States. Patients with IPNs have higher lung cancer risk than the general population, but most IPNs are benign, complicating evaluation. Moreover, 25.8% of transthoracic needle biopsies cause clinical complications. For this reason, IPN management standard of care (SOC) is conservative monitoring of nodule size and/or PET-CT to rule-out malignancy. However, PET-CT evaluation is complicated by diabetic hyperglycemia, inconsistent standardized uptake values (SUVs), and infectious etiology false positives. Liquid biopsies could aid diagnostic adjudication of IPNs but would require high sensitivity at small nodule sizes (≤3 cm) to improve upon PET-CT SOC. To the inventors' knowledge, only one non-PET-CT test is available for IPN malignancy determination, with an AUROC of 0.76 in a validation cohort (97% sensitive, 44% specific; PMID: 29496499), demonstrating the unmet need for innovation.

The inventors and others previously characterized intracellular, cancer type-specific communities of intratumoral microbiomes, whose genomes are detectable in circulation and comprise orthogonal biomarkers to human-derived molecules (PMID: 32214244; 32467386; 36179670). However, this work identified a small fraction of the total metagenomic content, either by enriching for microbial-specific amplicons or capturing trace amounts of DNA or RNA fragments with matches in publicly-available reference databases. Even after repeated rounds of sensitive host depletion and quality filtering, 98.8% of the −4.4 billion non-human DNA or RNA reads in The Cancer Genome Atlas (TCGA) failed to map to any known organism in the RefSeq30 (release 200) multi-domain database (PMID: 36179670), suggesting that substantial cancer-associated microbial diversity remained unexplored.

Beginning with the scientific problem of unmappable microbial reads, the methods and/or systems, described elsewhere herein, in some embodiments, may produce a tumor-centric reference database through metagenome assembly on 5187 whole-genome sequenced tumor tissue and/or blood-derived cancer samples. In some cases, the metagenomic assemblies may comprise de novo metagenomic assemblies. In some cases, the tumor-centric reference data may comprise up to about 1562 metagenomic bins. In some cases, the tumor-centric reference data may comprise at least about 1562 metagenomic bins. In some embodiments, these bins increase the median mapping rate of non-human DNA in TCGA by at least about 891-fold while simultaneously reducing the median mapping rates of reagent-based contaminants by at least about 7.6-fold compared to publicly available reference genomes. The methods and/or systems of the present, as explored elsewhere herein, in some embodiments, exploit these cancer-derived metagenomic bins to create two distinct multi-omic, multi-species tests for lung cancer (e.g., early stage) detection that combine bin-derived information with other data features (e.g., plasma proteins and clinical risk scores, described elsewhere herein) via predictive (e.g., machine learning) modeling. In some cases, a first model identifies lung cancer of a subject (e.g., a subject in an otherwise healthy population), and a second model may determine a malignancy status of e.g., a detected lung cancer or nodule IPN of the subject. In some cases, each model of the first and the second model show strong predictive performances in validation subsets of stage I disease with at least about 85% accuracy, specificity, sensitivity, precision, AUPR, AUROC, or any combination thereof predictive performance of the model(s). Thus, by examining unexplored microbial diversity, the instant disclosure shows and describes the practical utility of plasma-derived metagenomes to diagnose early-stage cancer and additionally demonstrate that the metagenomic bins serve as a pan-cancer (i.e., multi-cancer type, not restricted to lung cancer) database from which cancer-associated microbial biomarkers can be identified.

Aspects of the disclosure provided herein describe a method of determining a disease of a subject, comprising: receiving a biological sample, electronic medical record information, and one or more radiologic images of a subject; sequencing one or more nucleic acid molecules isolated from the biological sample thereby generating one or more nucleic acid molecule sequencing reads; and determining a disease of the subject as an output of a predictive model when the predictive model is provided the subject's one or more nucleic acid molecule sequencing reads, electronic medical record information, and data derived from one or more radiologic images as an input. In some embodiments, the one or more nucleic acid molecule sequencing reads comprise one or more microbial nucleic acid molecule sequencing reads, and wherein the predictive model is provided the one or more microbial nucleic acid molecule sequencing reads as an input. In some embodiments, the method further comprises identifying one or more protein biomarkers from the biological sample of the subject. In some embodiments, the predictive model is provided the one or more protein biomarkers from the biological sample of the subject. In some embodiments, the one or more protein biomarkers comprise carcinoembryonic antigen, osteopontin, cancer antigen 15-3, cancer antigen 19-9, cancer antigen 125, interleukin-8, prolactin, cytokeratin 19 fragment (CYFRA 21-1), MMP-9, sTNFRII, MMP-7, Resistin, MPO, MCP-1, GRO, sVEGFR2, sKDR, sFlk-1, VEGF-A, VEGF-C, VEGF-D, HGF, CRp, MIF, PDGF, AB/bb, RANTES, SAA, TNFRII, or a combination thereof. In some embodiments, the disease comprises cancer or non-cancerous diseased. In some embodiments, the biological sample comprises a liquid biopsy, a tissue biopsy, or a combination thereof. In some embodiments, the one or more radiologic images comprise x-ray, computed tomography (CT), low dose computed tomography, magnetic resonance imaging (MRI), ultrasound, positron emission tomography, fluoroscopy, angiography, or any combination thereof images. In some embodiments, the cancer comprises a tumor mass with a diameter less than 3 centimeters. In some embodiments, sequencing comprises amplicon-based 16S rRNA sequencing. In some embodiments, the amplicon-based 16S rRNA sequencing sequences the V6 region of the one or more nucleic acid molecules. In some embodiments, the one or more nucleic acid molecules comprise mammalian RNA, mammalian DNA, mammalian cell-free DNA, mammalian cell-free RNA, mammalian exosomal DNA, mammalian exosomal RNA, non-human RNA, non-human DNA, non-human cell-free DNA, non-human cell-free RNA, non-human exosomal DNA, non-human exosomal RNA, circulating tumor DNA, circulating tumor RNA, or any combination thereof. In some embodiments, the liquid biopsy comprises plasma, serum, whole blood, feces, urine, cerebral spinal fluid, saliva, sweat, tears, exhaled breath condensate, or any combination thereof. In some embodiments, the cancer comprises lung adenocarcinoma (LUAD), lung squamous cell carcinoma (LUSC), small cell lung cancer (SCLC), or any combination thereof. In some embodiments, the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the method further comprises calculating one or more features of the one or more radiologic images, wherein the one or more features of the one or more radiologic images are provided as an input to the predictive model. In some embodiments, the one or more features comprise Brock cancer probability score, lesion diameter, lesion spiculation, lesion solidity, or any combination thereof. In some embodiments, the method further comprises mapping or aligning the one or more nucleic acid sequencing reads to a genome database to determine one or more human, non-human, or a combination thereof features of the one or more nucleic acid sequencing reads. In some embodiments, the genome database comprises a microbial genome database. In some embodiments, the microbial genome database comprises a de novo metagenomic assembly. In some embodiments, the de novo metagenomic assembly is derived from biological samples representative of a health state. In some embodiments, the biological samples are tissue samples, liquid biopsy samples, or a combination thereof. In some embodiments, the liquid biopsy samples comprise plasma, serum, whole blood, feces, urine, cerebral spinal fluid, saliva, sweat, tears, exhaled breath condensate, or any combination thereof. In some embodiments, the health state comprises cancer, a pre-cancerous state, a non-malignant disease state, or a disease-free healthy state. In some embodiments, the microbial genome database comprises the RefSeq database, the Web of Life database, the Unified Human Gastrointestinal Genome (UHGG) database, or any combination thereof databases. In some embodiments, the genome database comprises a human genome database. In some embodiments, the predictive model comprises a machine learning model. In some embodiments, the predictive model comprises a neural network, convolutional neural network, logistic regression, random forest, supper vector machines, or any combination thereof. In some embodiments, the machine learning model comprises a machine learning classifier. In some embodiments, the machine learning model comprises a stacked machine learning model, one or more machine learning models, an ensemble machine learning model, or a combination thereof. In some embodiments, the predictive model is trained with leave one out verification. In some embodiments, the predictive model is configured to determine a stage of the cancer, anatomical origin of the cancer, or a combination thereof. In some embodiments, the stage of the cancer is stage I, stage II, stage III, or stage IV. In some embodiments, the method further comprises decontaminating the one or more nucleic acid molecule sequencing reads to produce one or more decontaminated nucleic acid molecule sequencing reads. In some embodiments, decontaminating comprises in silico decontamination, experimental control decontamination, or a combination thereof. In some embodiments, the predictive model determines the disease with an accuracy of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, sequencing comprises shotgun metagenomic sequencing, next generation sequencing, long read sequencing, or any combination thereof. In some embodiments, the method further comprises determining one or more features of the one or more nucleic acid molecule sequencing reads. In some embodiments, the one or more features of the one or more nucleic acid molecules comprises non-microbial taxonomic abundance, mammalian genomic coordinates, annotated genomic loci, mammalian functional gene and/or biochemical pathway abundances, or any combination thereof features and a number of sequencing reads associated with said one or more features. In some embodiments, the predictive model is configured to differentiate cancer and a non-cancerous disease of the subject. In some embodiments, the mapping or aligning is completed with Deblur, Bowtie2, Kraken, or any combination thereof.

Another aspect of the disclosure provided herein describes a method, comprising: receiving a biological sample, electronic medical record information, data derived from one or more radiologic images, and a corresponding disease of one or more subjects; sequencing one or more nucleic acid molecules isolated from the biological sample thereby generating one or more nucleic acid molecule sequencing reads; and identifying one or more features of the one or more nucleic acid molecule sequencing reads, electronic medical record information, and the data derived from the one or more radiologic images that correspond to the disease of the one or more subjects. In some embodiments, the one or more nucleic acid molecule sequencing reads comprise one or more microbial nucleic acid molecule sequencing reads, and wherein the predictive model is provided the one or more microbial nucleic acid molecule sequencing reads as an input. In some embodiments, identifying comprises aligning the one or more sequencing reads to a genome database. In some embodiments, the method further comprises training a predictive model with the one or more features of the nucleic acid molecule sequencing reads, electronic medical record information, and the data derived from the one or more radiologic images and the corresponding disease of the one or more subjects. In some embodiments, the disease comprises cancer or non-cancerous disease. In some embodiments, the method further comprises identifying one or more features of one or more protein biomarkers of the biological sample of the subject. In some embodiments, the one or more protein biomarkers comprise carcinoembryonic antigen, osteopontin, cancer antigen 15-3, cancer antigen 19-9, cancer antigen 125, interleukin-8, prolactin, cytokeratin 19 fragment (CYFRA 21-1)), MMP-9, sTNFRII, MMP-7, Resistin, MPO, MCP-1, GRO, sVEGFR2, sKDR, sFlk-1, VEGF-A, VEGF-C, VEGF-D, HGF, CRp, MIF, PDGF, AB/bb, RANTES, SAA, TNFRII, or a combination thereof. In some embodiments, the biological sample comprises a liquid biopsy, a tissue biopsy, or a combination thereof. In some embodiments, the one or more radiologic images comprise x-ray, computed tomography (CT), low dose computed tomography, magnetic resonance imaging (MRI), ultrasound, positron emission tomography, fluoroscopy, angiography, or any combination thereof images. In some embodiments, the cancer comprises a tumor mass with a diameter less than 3 centimeters. In some embodiments, sequencing comprises amplicon-based 16S rRNA sequencing. In some embodiments, the amplicon-based 16S rRNA sequencing sequences the V6 region of the one or more nucleic acid molecules. In some embodiments, the one or more nucleic acid molecules comprise mammalian RNA, mammalian DNA, mammalian cell-free DNA, mammalian cell-free RNA, mammalian exosomal DNA, mammalian exosomal RNA, non-human RNA, non-human DNA, non-human cell-free DNA, non-human cell-free RNA, non-human exosomal DNA, non-human exosomal RNA, circulating tumor DNA, circulating tumor RNA, or any combination thereof. In some embodiments, the liquid biopsy comprises plasma, serum, whole blood, feces, urine, cerebral spinal fluid, saliva, sweat, tears, exhaled breath condensate, or any combination thereof. In some embodiments, the cancer comprises lung adenocarcinoma (LUAD, lung squamous cell carcinoma (LUSC), small cell lung cancer (SCLC), or any combination thereof. In some embodiments, the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the one or more radiologic image features comprise Brock cancer probability score, lesion diameter, lesion spiculation, lesion solidity, or any combination thereof. In some embodiments, the method further comprises mapping or aligning the one or more nucleic acid sequencing reads to a genome database to determine one or more human, non-human, or a combination thereof features of the one or more nucleic acid sequencing reads. In some embodiments, the genome database comprises a microbial genome database. In some embodiments, the microbial genome database comprises a de novo metagenomic assembly. In some embodiments, the de novo metagenomic assembly is derived from biological samples representative of a health state. In some embodiments, the biological samples are tissue samples, liquid biopsy samples, or a combination thereof. In some embodiments, the liquid biopsy samples comprise plasma, serum, whole blood, feces, urine, cerebral spinal fluid, saliva, sweat, tears, exhaled breath condensate, or any combination thereof. In some embodiments, the health state comprises cancer, a pre-cancerous state, a non-malignant disease state, or a disease-free healthy state. In some embodiments, the microbial genome database comprises the RefSeq database, the Web of Life database, the Unified Human Gastrointestinal Genome (UHGG) database, or any combination thereof databases. In some embodiments, the genome database comprises a human genome database. In some embodiments, the predictive model comprises a machine learning model. In some embodiments, the predictive model comprises a neural network, convolutional neural network, logistic regression, random forest, supper vector machines, or any combination thereof. In some embodiments, the machine learning model comprises a machine learning classifier. In some embodiments, the machine learning model comprises a stacked machine learning model, one or more machine learning models, an ensemble machine learning model, or a combination thereof. In some embodiments, the predictive model is trained with leave one out verification. In some embodiments, the predictive model is configured to determine a stage of the cancer, anatomical origin of the cancer, or a combination thereof. In some embodiments, the stage of the cancer is stage I, stage II, or stage III, or stage IV. In some embodiments, the method further comprises decontaminating the one or more nucleic acid molecule sequencing reads to produce one or more decontaminated nucleic acid molecule sequencing reads. In some embodiments, decontaminating comprises in silico decontamination, experimental control decontamination, or a combination thereof. In some embodiments, the predictive model determines the disease with an accuracy of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, sequencing comprises shotgun sequencing, next generation sequencing, long read sequencing, or any combination thereof. In some embodiments, the method further comprising determining one or more features of the one or more nucleic acid molecule sequencing reads. In some embodiments, the one or more features of the one or more nucleic acid molecules comprises non-microbial taxonomic abundance, mammalian genomic coordinates, annotated genomic loci, mammalian functional gene and/or biochemical pathway abundances, or any combination thereof features, and a number of sequencing reads associated with said one or more features. In some embodiments, the predictive model is configured to differentiate cancer and a non-cancerous disease of the subject. In some embodiments, the mapping or aligning is completed with Deblur, Bowtie2, Kraken, or any combination thereof.

Another aspect of the disclosure provided herein describes a computer system configured to determine a disease of a subject, comprising: (a) one or more processors; and (b) a non-transient computer readable storage medium including software, wherein the software comprises executable instructions that, as a result of execution, cause the one or more processors of the computer system to: (i) receive one or more sequencing reads of a biological sample, electronic medical record information, and one or more images of a subject; and (ii) determine a disease of the subject as an output of a predictive model when the predictive model is provided the subject's one or more nucleic acid molecule sequencing reads, electronic medical record information, and data derived from one or more radiologic images as an input. In some embodiments, the one or more nucleic acid sequencing reads comprise one or more microbial nucleic acid molecule sequencing reads, and wherein the predictive model is provided the one or more microbial nucleic acid molecule sequencing reads as an input. In some embodiments, the disease comprises cancer or non-cancerous disease. In some embodiments, the biological sample comprises a tissue biopsy, liquid biopsy, or a combination thereof. In some embodiments, the executable instructions comprise receiving one or more protein biomarkers from the biological sample of the subject. In some embodiments, the predictive model is provided the one or more protein biomarkers from the biological sample of the subject. In some embodiments, the one or more protein biomarkers comprise carcinoembryonic antigen, osteopontin, or a combination thereof. In some embodiments, the predictive model is trained with the one or more features of the nucleic acid molecule sequencing reads, electronic medical record information, and the data derived from the one or more radiologic images and the corresponding disease of the one or more subjects. In some embodiments, the executable instructions comprise identifying one or more features of one or more protein biomarkers of the biological sample of the subject. In some embodiments, the one or more protein biomarkers comprise carcinoembryonic antigen, osteopontin, cancer antigen 15-3, cancer antigen 19-9, cancer antigen 125, interleukin-8, prolactin, cytokeratin 19 fragment (CYFRA 21-1), MMP-9, sTNFRII, MMP-7, Resistin, MPO, MCP-1, GRO, sVEGFR2, sKDR, sFlk-1, VEGF-A, VEGF-C, VEGF-D, HGF, CRp, MIF, PDGF, AB/bb, RANTES, SAA, TNFRII, or a combination thereof. In some embodiments, the one or more radiologic images comprise x-ray, computed tomography (CT), low dose computed tomography, magnetic resonance imaging (MRI), ultrasound, positron emission tomography, fluoroscopy, angiography, or any combination thereof images. In some embodiments, the cancer comprises a tumor mass with a diameter less than 3 centimeters. In some embodiments, the one or more nucleic acid molecule sequencing reads comprises one or more amplicon-based 16S rRNA sequencing reads. In some embodiments, the amplicon-based 16S rRNA sequencing reads comprise sequencing reads of the V6 region of the one or more nucleic acid molecules. In some embodiments, the one or more nucleic acid molecule sequencing reads comprise sequencing reads of mammalian RNA, mammalian DNA, mammalian cell-free DNA, mammalian cell-free RNA, mammalian exosomal DNA, mammalian exosomal RNA, non-human RNA, non-human DNA, non-human cell-free DNA, non-human cell-free RNA, non-human exosomal DNA, non-human exosomal RNA, circulating tumor DNA, circulating tumor RNA, or any combination thereof. In some embodiments, the liquid biopsy comprises plasma, serum, whole blood, feces, urine, cerebral spinal fluid, saliva, sweat, tears, exhaled breath condensate, or any combination thereof. In some embodiments, the cancer comprises lung adenocarcinoma (LUAD, lung squamous cell carcinoma (LUSC), small cell lung cancer (SCLC), or any combination thereof. In some embodiments, the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the one or more radiologic image features comprise Brock cancer probability score, lesion diameter, lesion spiculation, lesion solidity, or any combination thereof. In some embodiments, the executable instructions further comprising mapping or aligning the one or more nucleic acid sequencing reads to a genome database to determine one or more human, non-human, or a combination thereof features of the one or more nucleic acid sequencing reads. In some embodiments, the genome database comprises a microbial genome database. In some embodiments, the microbial genome database comprises a de novo metagenomic assembly. In some embodiments, the de novo metagenomic assembly is derived from biological samples representative of a health state. In some embodiments, the biological samples are tissue samples, liquid biopsy samples, or a combination thereof. In some embodiments, the liquid biopsy samples comprise plasma, serum, whole blood, feces, urine, cerebral spinal fluid, saliva, sweat, tears, exhaled breath condensate, or any combination thereof. In some embodiments, the health state comprises cancer, a pre-cancerous state, a non-malignant disease state, or a disease-free healthy state. In some embodiments, the microbial genome database comprises the RefSeq database, the Web of Life database, the Unified Human Gastrointestinal Genome (UHGG) database, or any combination thereof databases. In some embodiments, the genome database comprises a human genome database. In some embodiments, the predictive model comprises a machine learning model. In some embodiments, the predictive model comprises a neural network, convolutional neural network, logistic regression, random forest, supper vector machines, or any combination thereof. In some embodiments, the machine learning model comprises a machine learning classifier. In some embodiments, the machine learning model comprises a stacked machine learning model, one or more machine learning models, an ensemble machine learning model, or a combination thereof. In some embodiments, the predictive model is trained with leave one out verification. In some embodiments, the predictive model is configured to determine a stage of the cancer, anatomical origin of the cancer, or a combination thereof. In some embodiments, the stage of the cancer is stage I, stage II, stage III, or stage IV. In some embodiments, the executable instructions further comprise decontaminating the one or more nucleic acid molecule sequencing reads to produce one or more decontaminated nucleic acid molecule sequencing reads. In some embodiments, decontaminating comprises in silico decontamination, experimental control decontamination, or a combination thereof. In some embodiments, the predictive model determines the disease with an accuracy of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the one or more sequencing reads are generated by shotgun sequencing, next generation sequencing, long read sequencing, or any combination thereof. In some embodiments, the executable instructions further comprise determining one or more features of the one or more nucleic acid molecule sequencing reads. In some embodiments, the one or more features of the one or more nucleic acid molecules comprises non-microbial taxonomic abundance, mammalian genomic coordinates, annotated genomic loci, mammalian functional gene and/or biochemical pathway abundances, or any combination thereof features and a number of sequencing reads associated with said one or more features. In some embodiments, the predictive model is configured to differentiate cancer and a non-cancerous disease of the subject. In some embodiments, the mapping or aligning is completed with Deblur, Bowtie2, Kraken, or any combination thereof.

Another aspect of the disclosure provided herein describes a method of determining a disease of a subject, comprising: receiving a biological sample from a subject; sequencing one or more nucleic acid molecules of the biological sample thereby generating one or more nucleic acid molecule sequencing reads; and determining a disease of the subject as an output of a predictive model when the predictive model is provided the subject's one or more nucleic acid molecule sequencing reads, wherein the predictive model is trained with one or more nucleic acid molecule sequencing reads of one or more liquid biological samples and one or more tissue biological samples and corresponding disease of one or more subjects. In some embodiments, the one or more nucleic acid molecule sequencing reads comprise one or more microbial nucleic acid molecule sequencing reads, and wherein the predictive model is provided the one or more microbial nucleic acid molecule sequencing reads as an input. In some embodiments, the disease comprises cancer, non-cancerous diseased, or a combination thereof. In some embodiments, the method further comprises identifying one or more protein biomarkers from the biological sample of the subject. In some embodiments, the predictive model is provided the one or more protein biomarkers from the biological sample of the subject. In some embodiments, the one or more protein biomarkers comprise carcinoembryonic antigen, osteopontin, cancer antigen 15-3, cancer antigen 19-9, cancer antigen 125, interleukin-8, prolactin, cytokeratin 19 fragment (CYFRA 21-1), MMP-9, sTNFRII, MMP-7, Resistin, MPO, MCP-1, GRO, sVEGFR2, sKDR, sFlk-1, VEGF-A, VEGF-C, VEGF-D, HGF, CRp, MIF, PDGF, AB/bb, RANTES, SAA, TNFRII, or a combination thereof. In some embodiments, the cancer comprises a tumor mass with a diameter less than 3 centimeters millimeters. In some embodiments, the sequencing comprises amplicon-based 16S rRNA sequencing. In some embodiments, the amplicon-based 16S rRNA sequencing sequences the V6 region of the one or more nucleic acid molecules. In some embodiments, the one or more nucleic acid molecules comprise mammalian RNA, mammalian DNA, mammalian cell-free DNA, mammalian cell-free RNA, mammalian exosomal DNA, mammalian exosomal RNA, non-human RNA, non-human DNA, non-human cell-free DNA, non-human cell-free RNA, non-human exosomal DNA, non-human exosomal RNA, circulating tumor DNA, circulating tumor RNA, or any combination thereof. In some embodiments, the liquid biopsy comprises plasma, serum, whole blood, feces, urine, cerebral spinal fluid, saliva, sweat, tears, exhaled breath condensate, or any combination thereof. In some embodiments, the cancer comprises lung adenocarcinoma (LUAD, lung squamous cell carcinoma (LUSC), small cell lung cancer (SCLC), or any combination thereof. In some embodiments, the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the method further comprises mapping or aligning the one or more nucleic acid sequencing reads to a genome database to determine one or more human, non-human, or a combination thereof features of the one or more nucleic acid sequencing reads that are provided as an input to the predictive model. In some embodiments, the genome database comprises a microbial genome database. In some embodiments, the microbial genome database comprises a de novo metagenomic assembly. In some embodiments, the de novo metagenomic assembly is derived from biological samples representative of a health state. In some embodiments, the biological samples are tissue samples, liquid biopsy samples, or a combination thereof. In some embodiments, the liquid biopsy samples comprise plasma, serum, whole blood, feces, urine, cerebral spinal fluid, saliva, sweat, tears, exhaled breath condensate, or any combination thereof. In some embodiments, the health state comprises cancer, a pre-cancerous state, a non-malignant disease state, or a disease-free healthy state. In some embodiments, the microbial genome database comprises the RefSeq database, the Web of Life database, the Unified Human Gastrointestinal Genome (UHGG) database, or any combination thereof databases. In some embodiments, the genome database comprises a human genome database. In some embodiments, the predictive model comprises a machine learning model. In some embodiments, the predictive model comprises a neural network, convolutional neural network, logistic regression, random forest, supper vector machines, or any combination thereof. In some embodiments, the machine learning model comprises a machine learning classifier. In some embodiments, the machine learning model comprises a stacked machine learning model, one or more machine learning models, an ensemble machine learning model, or a combination thereof. In some embodiments, the predictive model is trained with leave one out verification. In some embodiments, the predictive model is configured to determine a stage of the cancer, anatomical origin of the cancer, or a combination thereof. In some embodiments, the stage of the cancer is stage I, stage II, stage III, or stage IV. In some embodiments, the method further comprises decontaminating the one or more nucleic acid molecule sequencing reads to produce one or more decontaminated nucleic acid molecule sequencing reads, wherein the one or more decontaminated nucleic acid molecules are provided to the predictive model as an input. In some embodiments, decontaminating comprises in silico decontamination, experimental control decontamination, or a combination thereof. In some embodiments, the predictive model determines the disease with an accuracy of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, sequencing comprises shotgun sequencing, next generation sequencing, long read sequencing, or any combination thereof. In some embodiments, the method further comprises determining one or more features of the one or more nucleic acid molecule sequencing reads. In some embodiments, the one or more features of the one or more nucleic acid molecules comprises non-microbial taxonomic abundance, mammalian genomic coordinates, annotated genomic loci, mammalian functional gene and/or biochemical pathway abundances, or any combination thereof features, and a number of sequencing reads associated with said one or more features. In some embodiments, the predictive model is configured to differentiate cancer and a non-cancerous disease of the subject. In some embodiments, mapping or aligning is completed with Deblur, PICRUSt2, Bowtie2, Kraken, or any combination thereof.

Another aspect of the disclosure provided herein describes a method of identifying one or more non-human genomic features, comprising: receiving one or more liquid biological samples, one or more tissue biological samples, and a corresponding disease of one or more subjects; sequencing one or more nucleic acid molecules of the one or more liquid biological samples and the one or more tissue biological samples thereby generating one or more sequencing reads; and identifying one or more non-human genomic features that correspond to the disease of the one or more subjects from the one or more sequencing reads. In some embodiments, the one or more nucleic acid molecule sequencing reads comprise one or more microbial nucleic acid molecule sequencing reads, and wherein the predictive model is provided the one or more microbial nucleic acid molecule sequencing reads as an input. In some embodiments, identifying comprises aligning or mapping the one or more sequencing reads to a genome database to determine one or more human, non-human, or a combination thereof features of the one or more nucleic acid sequencing reads. In some embodiments, the genome database comprises a microbial genome database. In some embodiments, the microbial genome database comprises a de novo metagenomic assembly. In some embodiments, the de novo metagenomic assembly is derived from biological samples representative of a health state. In some embodiments, the biological samples are tissue samples, liquid biopsy samples, or a combination thereof. In some embodiments, the liquid biopsy samples comprise plasma, serum, whole blood, feces, urine, cerebral spinal fluid, saliva, sweat, tears, exhaled breath condensate, or any combination thereof. In some embodiments, the health state comprises cancer, a pre-cancerous state, a non-malignant disease state, or a disease-free healthy state. In some embodiments, the microbial genome database comprises the RefSeq database, the Web of Life database, the Unified Human Gastrointestinal Genome (UHGG) database, or any combination thereof databases. In some embodiments, the method further comprises training a predictive model with the one or more non-human genomic features and the corresponding disease of the one or more subjects. In some embodiments, the disease comprises cancer or non-cancerous disease. In some embodiments, the method further comprises identifying one or more features of one or more protein biomarkers of the one or more liquid biological sample, one or more tissue biological samples, or a combination thereof. In some embodiments, the one or more protein biomarkers comprise carcinoembryonic antigen, osteopontin, cancer antigen 15-3, cancer antigen 19-9, cancer antigen 125, interleukin-8, prolactin, cytokeratin 19 fragment (CYFRA 21-1), MMP-9, sTNFRII, MMP-7, Resistin, MPO, MCP-1, GRO, sVEGFR2, sKDR, sFlk-1, VEGF-A, VEGF-C, VEGF-D, HGF, CRp, MIF, PDGF, AB/bb, RANTES, SAA, TNFRII, or a combination thereof. In some embodiments, the cancer comprises a tumor mass with a diameter less than 3 centimeters. In some embodiments, the sequencing comprises amplicon-based 16S rRNA sequencing. In some embodiments, the amplicon-based 16S rRNA sequencing sequences the V6 region of the one or more nucleic acid molecules. In some embodiments, the one or more nucleic acid molecules comprise mammalian RNA, mammalian DNA, mammalian cell-free DNA, mammalian cell-free RNA, mammalian exosomal DNA, mammalian exosomal RNA, non-human RNA, non-human DNA, non-human cell-free DNA, non-human cell-free RNA, non-human exosomal DNA, non-human exosomal RNA, circulating tumor DNA, circulating tumor RNA, or any combination thereof. In some embodiments, the liquid biological sample comprises plasma, serum, whole blood, urine, cerebral spinal fluid, saliva, sweat, tears, exhaled breath condensate, or any combination thereof. In some embodiments, the cancer comprises lung adenocarcinoma (LUAD, lung squamous cell carcinoma (LUSC), small cell lung cancer (SCLC), or any combination thereof. In some embodiments, the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the genome database comprises a human genome database. In some embodiments, the predictive model comprises a machine learning model. In some embodiments, the predictive model comprises a neural network, convolutional neural network, logistic regression, random forest, supper vector machines, or any combination thereof. In some embodiments, the machine learning model comprises a machine learning classifier. In some embodiments, the machine learning model comprises a stacked machine learning model, one or more machine learning models, an ensemble machine learning model, or a combination thereof. In some embodiments, the predictive model is trained with leave one out verification. In some embodiments, the predictive model is configured to determine a stage of the cancer, anatomical origin of the cancer, or a combination thereof. In some embodiments, the stage of the cancer is stage I, stage II, stage III, or stage IV. In some embodiments, the method further comprises decontaminating the one or more nucleic acid molecule sequencing reads to produce one or more decontaminated nucleic acid molecule sequencing reads. In some embodiments, decontaminating comprises in silico decontamination, experimental control decontamination, or a combination thereof. In some embodiments, the predictive model determines the disease with an accuracy of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, sequencing comprises shotgun sequencing, next generation sequencing, long read sequencing, or any combination thereof. In some embodiments, the method further comprises determining one or more features of the one or more nucleic acid molecule sequencing reads. In some embodiments, the one or more features of the one or more nucleic acid molecules comprises non-microbial taxonomic abundance, mammalian genomic coordinates, annotated genomic loci, mammalian functional gene and/or biochemical pathway abundances, or any combination thereof features and a number of sequencing reads associated with said one or more features. In some embodiments, the predictive model is configured to differentiate cancer and a non-cancerous disease of the subject. In some embodiments, mapping or aligning is completed with Deblur, PICRUSt2, Bowtie2, Kraken, or any combination thereof.

Another aspect of the disclosure provided herein describes a computer system configured to determine a disease of a subject, comprising: (a) one or more processors; and (b) a non-transient computer readable storage medium including software, wherein the software comprises executable instructions that, as a result of execution, cause the one or more processors of the computer system to: (i) receive one or more sequencing reads of a biological samples of a subject; and (ii) determining a disease of the subject as an output of a predictive model when the predictive model is provided the subject's one or more nucleic acid molecule sequencing reads, wherein the predictive model is trained with one or more nucleic acid molecule sequencing reads of one or more liquid biological samples and one or more tissue biological samples and corresponding disease of one or more subjects. In some embodiments, the one or more nucleic acid molecule sequencing reads comprise one or more microbial nucleic acid molecule sequencing reads, and wherein the predictive model is provided the one or more microbial nucleic acid molecule sequencing reads as an input. In some embodiments, the disease comprises cancer or non-cancerous disease. In some embodiments, the disease comprises cancer or non-cancerous disease. In some embodiments, the executable instructions comprise receiving one or more protein biomarkers from the biological sample of the subject. In some embodiments, the predictive model is provided the one or more protein biomarkers from the biological sample of the subject. In some embodiments, the executable instructions comprise identifying one or more features of one or more protein biomarkers of the biological sample of the subject. In some embodiments, the one or more protein biomarkers comprise carcinoembryonic antigen, osteopontin, cancer antigen 15-3, cancer antigen 19-9, cancer antigen 125, interleukin-8, prolactin, cytokeratin 19 fragment (CYFRA 21-1), MMP-9, sTNFRII, MMP-7, Resistin, MPO, MCP-1, GRO, sVEGFR2, sKDR, sFlk-1, VEGF-A, VEGF-C, VEGF-D, HGF, CRp, MIF, PDGF, AB/bb, RANTES, SAA, TNFRII, or a combination thereof. In some embodiments, the cancer comprises a tumor mass with a diameter less than 3 centimeters. In some embodiments, the one or more nucleic acid molecule sequencing reads comprises one or more amplicon-based 16S rRNA sequencing reads. In some embodiments, the amplicon-based 16S rRNA sequencing reads comprise sequencing reads of the V6 region of the one or more nucleic acid molecules. In some embodiments, the one or more nucleic acid molecule sequencing reads comprise sequencing reads of mammalian RNA, mammalian DNA, mammalian cell-free DNA, mammalian cell-free RNA, mammalian exosomal DNA, mammalian exosomal RNA, non-human RNA, non-human DNA, non-human cell-free DNA, non-human cell-free RNA, non-human exosomal DNA, non-human exosomal RNA, circulating tumor DNA, circulating tumor RNA, or any combination thereof. In some embodiments, the liquid biological sample comprises plasma, serum, whole blood, urine, cerebral spinal fluid, saliva, sweat, tears, exhaled breath condensate, or any combination thereof. In some embodiments, the cancer comprises lung adenocarcinoma (LUAD, lung squamous cell carcinoma (LUSC), small cell lung cancer (SCLC), or any combination thereof. In some embodiments, the cancer comprises: acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lymphoid neoplasm diffuse large B-cell lymphoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, or any combination thereof. In some embodiments, the executable instructions further comprising mapping or aligning the one or more nucleic acid sequencing reads to a genome database to determine one or more human, non-human, or a combination thereof features of the one or more nucleic acid sequencing reads. In some embodiments, the genome database comprises a microbial genome database. In some embodiments, the microbial genome database comprises a de novo metagenomic assembly. In some embodiments, the de novo metagenomic assembly is derived from biological samples representative of a health state. In some embodiments, the biological samples are tissue samples, liquid biopsy samples, or a combination thereof. In some embodiments, the liquid biopsy samples comprise plasma, serum, whole blood, feces, urine, cerebral spinal fluid, saliva, sweat, tears, exhaled breath condensate, or any combination thereof. In some embodiments, the health state comprises cancer, a pre-cancerous state, a non-malignant disease state, or a disease-free healthy state. In some embodiments, the microbial genome database comprises the RefSeq database, the Web of Life database, the Unified Human Gastrointestinal Genome (UHGG) database, or any combination thereof databases. In some embodiments, the genome database comprises a human genome database. In some embodiments, the predictive model comprises a machine learning model. In some embodiments, the predictive model comprises a neural network, convolutional neural network, logistic regression, random forest, supper vector machines, or any combination thereof. In some embodiments, the machine learning model comprises a machine learning classifier. In some embodiments, the machine learning model comprises a stacked machine learning model, one or more machine learning models, an ensemble machine learning model, or a combination thereof. In some embodiments, the predictive model is trained with leave one out verification. In some embodiments, the predictive model is configured to determine a stage of the cancer, anatomical origin of the cancer, or a combination thereof. In some embodiments, the stage of the cancer is stage I, stage II, stage III, or stage IV. In some embodiments, the executable instructions further comprise decontaminating the one or more nucleic acid molecule sequencing reads to produce one or more decontaminated nucleic acid molecule sequencing reads. In some embodiments, decontaminating comprises in silico decontamination, experimental control decontamination, or a combination thereof. In some embodiments, the predictive model determines the disease with an accuracy of at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%. In some embodiments, the one or more sequencing reads are generated by shotgun sequencing, next generation sequencing, long read sequencing, or any combination thereof. In some embodiments, the executable instructions further comprise determining one or more features of the one or more nucleic acid molecule sequencing reads. In some embodiments, the one or more features of the one or more nucleic acid molecules comprises non-microbial taxonomic abundance, mammalian genomic coordinates, annotated genomic loci, mammalian functional gene and/or biochemical pathway abundances, or any combination thereof features and a number of sequencing reads associated with said one or more features. In some embodiments, the predictive model is configured to differentiate cancer and a non-cancerous disease of the subject. In some embodiments, the mapping or aligning is completed with Deblur, PICRUSt2, Bowtie2, Kraken, or any combination thereof.

Aspects of the disclosure described herein, in some embodiments, describe a method of determining a disease of a subject, comprising: (a) receiving a biological sample, electronic medical record information, and radiologic data of a subject; (b) sequencing a plurality of non-human nucleic acid molecules of the biological sample thereby generating a plurality of microbial sequencing reads; and (c) processing the plurality of microbial sequencing reads, electronic medical record information, and radiologic data with a trained predictive model, thereby determining the disease of the subject with at least about 80% accuracy, wherein the trained predictive model is trained with a plurality of microbial abundances and corresponding cancer type, wherein the trained predictive model comprises a first predictive model and a second predictive model, and wherein the first predictive model processes the plurality of microbial sequencing reads, electronic medical record information, and radiologic data, and wherein the second trained predictive model processes an output of the first predictive model. In some embodiments, the biological sample comprises a liquid biopsy. In some embodiments, the liquid biopsy comprises plasma, serum, whole blood, urine, feces, cerebral spinal fluid, saliva, sweat, tears, exhaled breath condensate, or any combination thereof. In some embodiments, the sequencing comprises shotgun sequencing. In some embodiments, the method further comprises receiving a concentration of one or more plasma proteins. In some embodiments, the trained predictive model comprises one or more machine learning models. In some embodiments, the method further comprises aligning a plurality of nucleic acid molecules sequencing reads of the biological sample with a human reference genome to identify a plurality of non-human nucleic acid molecule sequencing reads. In some embodiments, the method further comprises aligning the plurality of non-human nucleic acid molecule sequencing reads to a database of microbial genomes to identify the plurality of microbial sequencing reads. In some embodiments, the database comprises a de novo metagenomic assembly comprising genomic contigs. In some embodiments, the genomic contigs comprise one or more metagenomic bins. In some embodiments, aligning the plurality of non-human nucleic acid molecule sequencing reads to the de novo metagenomic assembly produces an aligned bin abundances of the plurality of non-human nucleic acid molecule sequencing reads. In some embodiments, the trained predictive model is configured to process the aligned bin abundances of the subject's plurality of non-human nucleic acid molecule sequencing reads. In some embodiments, the sequencing comprises amplicon-based 16S rRNA sequencing of a plurality of nucleic acid molecules of the biological sample. In some embodiments, the amplicon-based 16S rRNA sequencing reads comprise sequencing reads of the V6 region of the plurality of nucleic acid molecules of the biological sample. In some embodiments, the method further comprises determining one or more features of the radiologic data, wherein the one or more features of the radiologic data are processed by the trained predictive model. In some embodiments, the one or more features of the radiologic data comprise Brock cancer probability score, cancer lesion diameter, cancer lesion spiculation, cancer lesion solidity, or any combination thereof. In some embodiments, the disease comprises cancer. In some embodiments, the cancer comprises a tumor mass with a diameter less than about 3 centimeters or less than about 8 millimeters. In some embodiments, the trained predictive model is configured to determine a stage of the cancer, anatomical origin of the cancer, or a combination thereof. In some embodiments, determining the disease of the subject comprises distinguishing between cancer and a non-cancerous disease of the subject.

Aspects of disclosure described herein, in some embodiments, describe a system configured to determine a disease of a subject, comprising: (a) one or more processors; and (b) a non-transient computer readable storage medium including software, wherein the software comprises executable instructions that, as a result of execution, cause the one or more processors of the computer system to: (i) receive a plurality of non-human nucleic acid molecule sequencing reads of a biological sample, electronic medical record information, and radiologic data of a subject; and (ii) process a plurality of microbial nucleic acid molecule sequencing reads of the plurality of non-human nucleic acid molecule sequencing reads, electronic medical record information, and radiologic data of the subject with a trained predictive model, thereby determining the disease of the subject with at least about 80% accuracy, wherein the trained predictive model is trained with a plurality of microbial abundances and corresponding cancer type, wherein the trained predictive model comprises a first predictive model and a second predictive model, and wherein the first predictive model processes the plurality of microbial nucleic acid molecule sequencing reads, electronic medical record information, and radiologic data, and wherein the second predictive model processes an output of the first predictive model. In some embodiments, the biological sample comprises a liquid biopsy. In some embodiments, the liquid biopsy comprises plasma, serum, whole blood, urine, feces, cerebral spinal fluid, saliva, sweat, tears, exhaled breath condensate, or any combination thereof. In some embodiments, the plurality of microbial nucleic acid molecule sequencing reads are generated by shotgun sequencing. In some embodiments, the executable instructions comprises receiving a concentration of one or more plasma proteins. In some embodiments, the trained predictive model comprises one or more machine learning models. In some embodiments, the executable instructions cause the one or more processors to align a plurality of nucleic acid molecule sequencing reads of the biological sample with a human reference genome library to identify the plurality of non-human nucleic acid molecule sequencing reads. In some embodiments, the executable instructions cause the one or more processors to align the plurality of non-human nucleic acid molecule sequencing reads to a database of microbial genomes to identify the plurality of microbial sequencing reads. In some embodiments, the database comprises a de novo metagenomic assembly comprising genomic contigs. In some embodiments, the genomic contigs comprise one or more metagenomic bins. In some embodiments, the executable instructions cause the one or more processors to align the plurality of non-human nucleic acid molecule sequencing reads to the de novo metagenomic assembly to produce an aligned bin abundances of the plurality of non-human nucleic acid molecule sequencing reads. In some embodiments, the trained predictive model is configured to process the aligned bin abundances of the subject's plurality of non-human nucleic acid molecule sequencing reads. In some embodiments, the executable instructions cause the one or more processors to receive a plurality of amplicon-based 16S rRNA sequencing reads of the plurality of nucleic acid molecules of the biological sample. In some embodiments, the plurality of amplicon-based 16S rRNA sequencing reads comprise sequencing reads of a V6 region of the plurality of nucleic acid molecules of the biological sample. In some embodiments, the executable instructions cause the one or more processors to determine one or more features of the radiologic data, wherein the one or more features of the radiologic data are processed by the trained predictive model. In some embodiments, the one or more features of the radiologic data comprise Brock cancer probability score, cancer lesion diameter, cancer lesion spiculation, cancer lesion solidity, or any combination thereof. In some embodiments, the disease comprises cancer. In some embodiments, the cancer comprises a tumor mass with a diameter up to about 3 centimeters or up to about 8 millimeters. In some embodiments, the trained predictive model is configured to determine a stage of the cancer, anatomical origin of the cancer, or a combination thereof. In some embodiments, determining the disease of the subject comprises distinguishing between cancer and a non-cancerous disease of the subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a flow diagram of the datatype and data stream structure for stacked machine learning training, as described in some embodiments herein.

FIG. 2 shows a flow diagram of metagenomic data isolated and/or identified from human subjects, as described in some embodiments herein.

FIG. 3 shows a flow diagram for proteomic data derived from human subjects, as described in some embodiments herein.

FIG. 4 shows a flow diagram of datatypes derived from human subject samples and human subject medical records used to train and/or develop diagnostic classifiers, as described in some embodiments herein.

FIG. 5 shows a flow diagram of clinic-proteo-metagenomic features used to generate a lung cancer classifier, as described in some embodiments herein.

FIG. 6 shows a diagram of a computer system configured to implement the methods of the disclosure, as described in some embodiments herein.

FIGS. 7A-7K show experimental data and graphs of metagenomic features that broadly discriminate treatment-naïve lung cancer and healthy samples across diverse lung cancer histotypes and stages using stacked machine learning, as described in some embodiments herein.

FIGS. 8A-8H show experimental data and graphs of using metagenomic plasma biomarkers to discriminate between lung cancer and lung disease and the development of metagenomic bins to enhance discriminatory performance, as described in some embodiments herein.

FIGS. 9A-9M show experimental data and graphs of the performance of metagenomic bin-based pan-cancer classifier diagnosing the presence and type of cancer in blood and plasma of two independent cohorts, as described in some embodiments herein.

FIGS. 10A-10I show experimental data, graphs, and/or workflow diagrams for the development and validation of a clinic-proteo-metagenomic classifier for lung nodule malignancy determination, as described in some embodiments herein.

FIGS. 11A-11C show experimental data and graphs of decontamination and aggregate genomic coverage of plasma-derived microbiomes in a sample cohort, as described in some embodiments herein.

FIGS. 12A-12D show experimental data and graphs of batch correction analyses for the cancer genome atlas (TCGA) biological samples' microbiome using metagenomic bin features, as described in some embodiments herein.

FIGS. 13A-13E show experimental data and graphs of TCGA classifier performance for cancer discrimination using metagenomic bins between and among cancer types, as described in some embodiments herein.

FIGS. 14A-14B show experimental data and graphs of control analyses verifying TCGA tissue-based classifier performance by comparing machine learning models built with scrambled metadata or shuffled samples, as described in some embodiments herein.

FIGS. 15A-15C show experimental data and graphs of control analyses verifying TCGA blood-based classifiers performance and further machine learning using blood samples from low-stage cancers or comparing primary tumors from low and high clinical stages, as described in some embodiments herein.

FIGS. 16A-16F show experimental data and graphs of TCGA classifier performance using subset raw data with metagenomic bin abundances for primary tumor cancer type discrimination, as described in some embodiments herein.

FIGS. 17A-17F show experimental data and graphs of raw data control analyses to verify TCGA primary tumor classifier performances, as described in some embodiments herein.

FIGS. 18A-18D show experimental data and graphs of TCGA classifier performance using subset raw data with metagenomic bins and control analyses for primary tumor versus adjacent normal tissue discrimination, as described in some embodiments herein.

FIGS. 19A-19H show experimental data and graphs of TCGA blood classifier performance using subset, raw, metagenomic bin abundances for cancer discrimination and control analyses, as described in some embodiments herein.

FIGS. 20A-20E show experimental data and graphs of metagenomic bin alpha diversity across TCGA primary tumors, as described in some embodiments herein.

FIGS. 21A-21E show experimental data and graphs of classical metagenomic analysis of cancer type specificity in TCGA primary tumor samples when using raw metagenomic bin abundances, as described in some embodiments herein.

FIGS. 22A-22G show experimental data and graphs of differential abundance of metagenomic bins among primary tumor cancer types in TCGA, as described in some embodiments herein.

FIGS. 23A-23E show experimental data and graphs of metagenomic bin alpha diversity across TCGA blood samples, as described in some embodiments herein.

FIGS. 24A-24E show experimental data and graphs of classical metagenomic analyses showing cancer type specificity in TCGA blood samples when using raw metagenomic bin abundances, as described in some embodiments herein.

FIGS. 25A-25F show experimental data and graphs of differential abundance of metagenomic bins among blood samples and their concomitant cancer types in TCGA, as described in some embodiments herein.

FIGS. 26A-26C show experimental data and graphs of diagnostic performance of metagenomes, proteins, and amplicons in a Comprehensive Oncobiome analysis for Diagnostic Identification of Cancer in Early Stages (CODICES) cohort of subjects, as described in some embodiments herein.

FIG. 27 shows experimental data and graphs of diagnostic performance of metagenomic bins in the CODICES cohort, as described in some embodiments herein.

FIG. 28 shows a flow diagram of data types derived from human subject samples and human subject medical records used to train and/or develop diagnostic classifiers, as described in some embodiments herein.

FIGS. 29A-29N show experimental data and graphs of diagnostic performance of metagenomes and DNA fragmentomic (nucleotide frequencies) analyses derived from publicly available cell-free DNA datasets, as described in some embodiments herein.

FIGS. 30A-30F show experimental data and graphs of lung cancer vs. healthy diagnostic performances of metagenomes, plasma proteins and DNA fragmentomic (nucleotide frequencies) analyses derived from the CODICES cohort, as described in some embodiments herein.

FIGS. 31A-31D, show experimental data and graphs of lung cancer vs. lung disease performances of metagenomes, plasma proteins and DNA fragmentomic (nucleotide frequencies) analyses derived from the CODICES cohort, as described in some embodiments herein.

FIGS. 32A-32D shows improved whole genome and RNA sequencing mapping rates to the metagenomic assembly bins compared to the publicly available RefSeq206 genome database.

### DETAILED DESCRIPTION

The disclosure describes, in some embodiments, a method of determining, discrimination, and/or differentiating a malignant disease (e.g., malignant pulmonary neoplasms alternatively referred to as tumors) from a benign disease by analyzing and/or assays a combination of two or more data types of a patient sample. In some cases, the disclosure describes methods and/or systems for determining and/or diagnosing a disease. In some instances, the disease may comprise cancer. In some cases, the disease may comprise a non-cancerous disease. In some cases, the methods and/or systems described elsewhere herein may differentiate and/or distinguish a cancerous and non-cancerous disease of a subject. In some cases, the cancer may comprise a tumor mass with a diameter less than about 3 centimeters or less than about 8 millimeters. In some embodiments, the data types of the patient sample may comprise one or more analyte types of a patient sample. The one or more analyte types may comprise the presence and/or abundance of one or more nucleic acid molecules of non-human origin (e.g., microbial, bacterial, virus, and/or fungi) of a liquid based (e.g., blood-derived) patient sample, non-microbial human derived analytes (e.g., proteins, human genomic nucleic acid molecules), or a combination thereof. In some cases, the liquid biopsy may comprise plasma, serum, whole blood, urine, feces, cerebral spinal fluid, saliva, sweat, tears, exhaled breath condensate, or any combination thereof. In some cases, the data types may comprise a patient medical history and/or diagnostic data type. The description provides, in some embodiments, a method and/or system configured and/or capable of detecting and/or determining a presence of early clinical stage cancers (e.g., lung cancer of Stage I and II) in an asymptomatic subject(s) at high risk for lung cancer due to the subject's smoking history. For example, such subjects may comprise subjects who fall within the United States Preventative Services Task Force recommendations for annual low-dose computed tomography screening: adults aged 50-80 years who have a 20 pack-year smoking history and currently smoke or have quit smoking within the last 15 years. The method(s), as described elsewhere herein, may comprise training one or more predictive models (e.g., machine learning algorithms) on datasets (e.g., feature tables), where the datasets comprise the aforementioned data types and thereby identifies disease (e.g., cancer)-correlative patterns among these data types. Specifically, the methods and/or systems, as described elsewhere herein, may train one or more predictive models with analyte types of microbial abundances (relative or absolute) obtained from sequencing (e.g., determining microbial read counts with next generation sequencing) one or more microbial nucleic acid molecules alone or in combination with measured plasma protein concentrations. In some cases, sequencing may comprise shotgun sequencing. In some embodiments, the training data set may comprise microbial abundances, measured plasma protein, human genomic information (e.g., DNA fragmentation patterns or chromosomal copy number variations), cancer risk scores calculated on the basis of information obtained from the patient medical chart, or any combination thereof. In some embodiments, the methods and/or systems, described elsewhere herein may train a predictive model (e.g., a multi-modal model) with one or more data types to produce a trained diagnostic model (e.g., trained multi-modal diagnostic model).

In some cases, the methods and/or systems described elsewhere herein, achieve the at least about 70% or at least about 0.7 performance metrics (e.g., accuracy, sensitivity, specificity, NPV, PPV, AUROC, and/or AUPR) of the predictive model in determining, diagnosing, and/or detecting cancer of a subject from one or more datatypes. In some cases, the use of an analyte type of nucleic acids of non-human origin when training the one or more predictive models, described elsewhere herein, may improve performance of trained models in discriminating between a first disease states and a second disease state of the lung e.g., the presence of lung cancer nodules and non-cancer lung nodules. In some cases, the non-cancer lung nodules may arise from etiologies including sarcoidosis, interstitial pulmonary fibrosis, bronchiectasis, pneumonia, chronic obstructive pulmonary disease, and/or hamartoma. Traditionally, such nodule-bearing condition may be differentiated from bona fide pulmonary malignancies by invasive intrathoracic biopsy followed by histopathological examination. The methods and/or systems described elsewhere herein present methods and/or systems that superior to a typical pathology report in examining e.g., an intrathoracic tissue biopsy since the methods and/or systems of the disclosure do not rely upon an observed subjective measure of tissue structure, cellular atypia, or any other subjective measure traditionally used to diagnose cancer. In some embodiments, the methods and/or systems, may instead rely on a measured quantified presence and/or abundance of one or more data types. In some cases, the methods and/or systems may exhibit an increase in a performance metric, as described elsewhere herein, by narrowing features on solely on microbial sources rather than modified human (i.e., cancerous) sources, which are modified often at extremely low frequencies in a background of ‘normal’ human sources. Moreover, the methods and/or systems of the disclosure may be conducted with and/or on blood derived samples, which is minimally invasive sample compared to e.g., an invasive biopsy, and therefore poses little risk to the patient and can be repeated longitudinally and for a low cost.

In some embodiments, the methods and/or systems described elsewhere herein, utilize, analyze, and/or train predictive models with metagenomic assembly generated from the non-human sequencing reads obtained from tumor whole genome sequencing data. In some cases, the metagenomic assemblies may comprise de novo metagenomic assemblies. In some cases, the methods, by using the non-human component of the whole genome sequencing data, may determine microbial constituents of a tumor type and use these constituents as a reference database for subsequent NGS sequencing read alignments. The metagenomic assemblies utilized by the methods and/or systems, as described elsewhere herein, are derived, in part, from treatment naïve biopsied tumor samples in The Cancer Genome Atlas (TCGA) and thereby represent diverse metagenomes from more than 30 distinct cancer types and/or (sub)types In some embodiments, cell-free DNA sequencing reads from a subject (e.g., a patient) sample are computationally aligned to one or more tumor-derived metagenomes, as described elsewhere herein, to identify the presence and/or abundance of tumor-associated microbial features. Those sample-associated microbial abundances may then be joined with other data types e.g., plasma protein concentrations and/or clinical data features—to form a multi-modal feature set that is inputted to a trained diagnostic model to produce a likelihood of cancer score. This score can be used by a medical care personnel and/or clinician to determine if a subsequent more invasive biopsy, e.g., a thoracic biopsy may be required or if continued non-invasive monitoring of the lung nodule via radiomic imaging e.g., as low dose computed tomography and/or further liquid biopsy based analysis and/or assays methods, as described elsewhere herein.

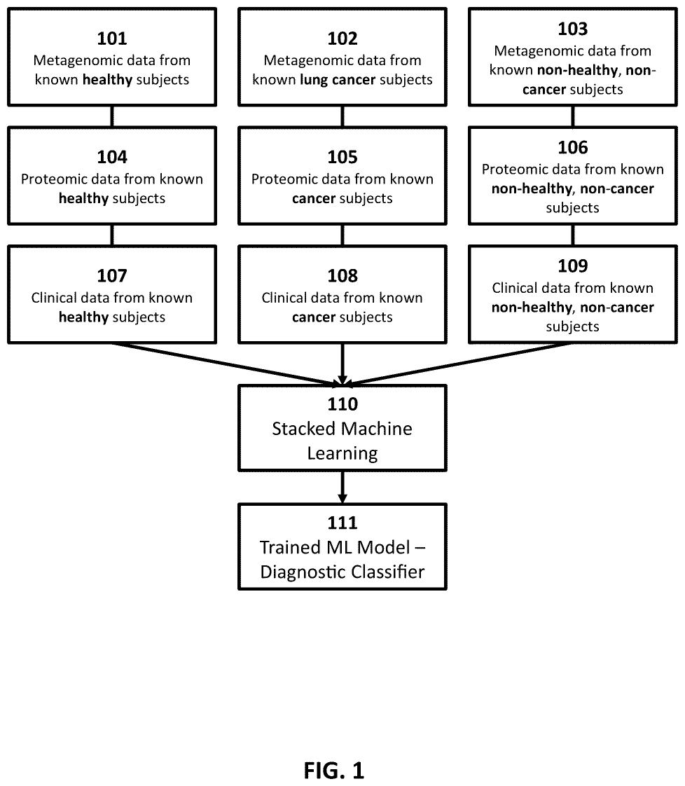

In some embodiments, metagenomic data may be combined with other data types to form a multi-modal input dataset for training a predictive model (e.g., one or more machine learning algorithms). In some embodiments a trained diagnostic model is generated using multi-modal data derived from one or more patients and/or subjects with known health histories. For example, in the case of lung cancer, metagenomic data from known healthy subjects 101, subjects with lung cancer 102, and subjects with non-cancer lung conditions (e.g., sarcoidosis, interstitial pulmonary fibrosis, bronchiectasis, pneumonia, chronic obstructive pulmonary disease, etc.) 103, may be combined with proteomic data (104-106) and clinical data (107-109) from the same subjects and used as a training dataset for an ensemble (“stacked”) predictive model (e.g., a stacked machine learning algorithm) 110 capable of learning from data containing both numerical and categorical data types. In some embodiments, predictions of a stacked predictive model from distinct model types (e.g., logistic regression, random forest, and support vector machines) may be combined to produce a single final predictive model 111, as shown in FIG. 1. The result of training this stacked predictive model may comprise a diagnostic classifier that can be used to differentiate one or more subject with cancer vs. healthy subjects and/or one or more subjects with cancer vs. non-cancer (i.e., lung disease) subjects based on analysis of one or more data types of the subjects' samples that have not been previously used in training the predictive model.

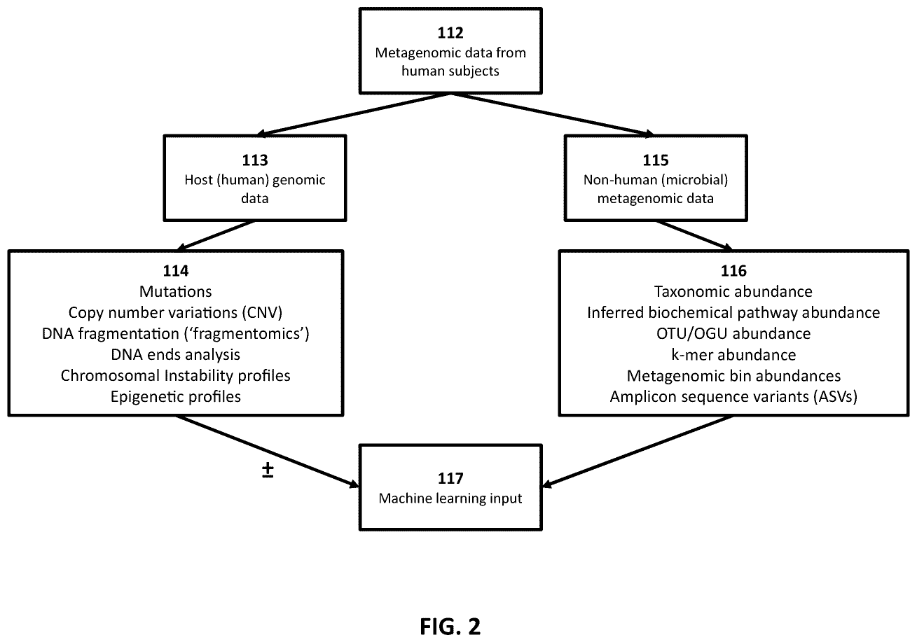

In some embodiments, the metagenomic data (101-103, FIG. 1; and/or 112, FIG. 2) may be derived from one or more training and/or test subjects' biological samples. In some embodiments the one or more training and/or test subjects' biological samples may comprise human 113 and non-human 115 nucleic acid molecules. In some cases, one or more sequences of the human 113 and/or non-human 115 nucleic acid molecules may be determined by sequencing. In some cases, sequencing may comprise next generation sequencing. In some cases, sequencing may comprise shotgun sequencing. In some cases, sequencing the one or more sequences of human 113 and/or non-human 115 nucleic acid molecules may generate and/or produce one or more sequencing reads of the human 113 and/or non-human 115 nucleic acid molecules. In some cases, one or more genomic analyses and/or assays, as described elsewhere herein, may be conducted and/or applied to the human 113 sequencing reads to yield human genomic feature sets for subsequent predictive model learning, analysis, and/or training 117. For example, human genomic 114 analyses may comprise the detection of cancer-associated DNA mutations, copy number variations, DNA fragmentation profiles, DNA ends analysis (e.g., the analysis of nucleotide frequencies at the ends of DNA fragments), chromosomal instability profiles and epigenetic profiles (e.g., DNA methylation patterns), or any combination thereof. In some embodiments, DNA fragmentation (e.g., DNA ends analysis) analysis may provide a nucleotide frequency of one or more nucleotide bases. In some instances, a 100-base pair (bp) nucleic acid molecule sequence read length, may comprise about 1 to about 30 bases analyzed for nucleotide frequency. In some cases, a 100 bp nucleic acid molecule sequence read length, may comprise about 1 to about 25 bases analyzed for nucleotide frequency. In some cases, nucleotide frequency comprises the occurrence of a given nucleotide in the base segments analyzed. In some cases, at least 3 nucleotide bases are analyzed for a nucleic acid molecule sequence. In some embodiments, one or more analysis and/or assays can be performed on the non-human component 115 of a metagenomic dataset to produce feature tables for predictive model learning, training, and/or analysis. In some cases, the non-human (e.g., microbial) genomic analysis and/or assays 116 may comprise include determining taxonomic abundance of microbes in a sample; inferring the abundance of biochemical pathways represented by the identified microbes; aligning the sequencing reads to metagenomic assemblies (‘bins’) to obtain bin abundances; determining the amplicon sequence variants presented in a targeted amplicon sequencing dataset; or any combination thereof. In some cases, a plurality of nucleic acid molecules of a biological sample (e.g., a liquid biopsy) may be aligned with a human reference genome to identify a plurality of non-human nucleic acid molecule sequencing reads. In some instances, the non-human nucleic acid molecule sequencing reads may be aligned to a database of microbial genomes to identify a plurality of microbial sequencing reads. In some cases, the database of microbial genomes may comprise a de novo metagenomic assembly comprising genomic contigs. In some cases, the genomic contigs may comprise one or more metagenomic bins. In some embodiments, the plurality of non-human nucleic acid molecule sequencing reads may be aligned to the de novo metagenomic assembly to produce an aligned bin abundance for the plurality of non-human nucleic acid molecule sequencing reads. In some cases, one or more predictive models, as described elsewhere herein, may process the aligned bin abundances of one or more subjects to determine a disease of the subject, as described elsewhere herein. In some cases, the targeted amplificon sequencing dataset may comprise an amplicon-based 16S rRNA sequencing read dataset of a plurality of nucleic acid molecules of a biological sample. In some cases, the amplicon-based 16S rRNA sequencing reads may comprise sequencing reads of a V6 region of a plurality of nucleic acid molecules of a biological sample of a subject, described elsewhere herein.

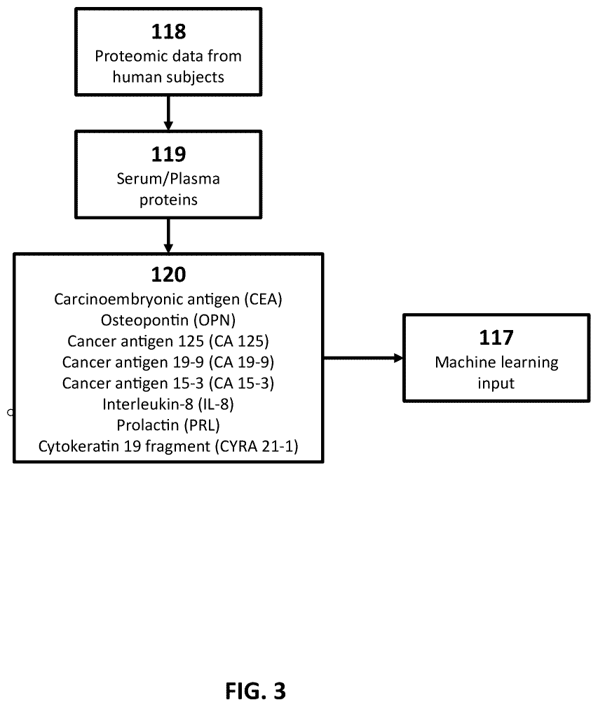

In some embodiments, the proteomic data (104-106, FIG. 1; 118, FIG. 3) derived from one or more training and/or test subjects' biological samples may comprise blood-derived serum or plasma proteins 119. In some embodiments, in the plasma proteins 120 may comprise carcinoembryonic antigen (CEA), osteopontin (OPN), cancer antigen 125 (CA 125), cancer antigen 19-9 (CA 19-9), cancer antigen 15-3 (CA 15-3), interleukin-8 (IL-8), prolactin (PRL) and cytokeratin 19 fragment (CYRA 21-1). The concentration of these plasma proteins (typically ng/mL) can be determined via immunoassay, as detailed herein, and used as input 117—alongside other data types (e.g., FIG. 1)—to train a predictive model (e.g., a machine learning algorithm) and/or to be analyzed by a previously trained predictive model.

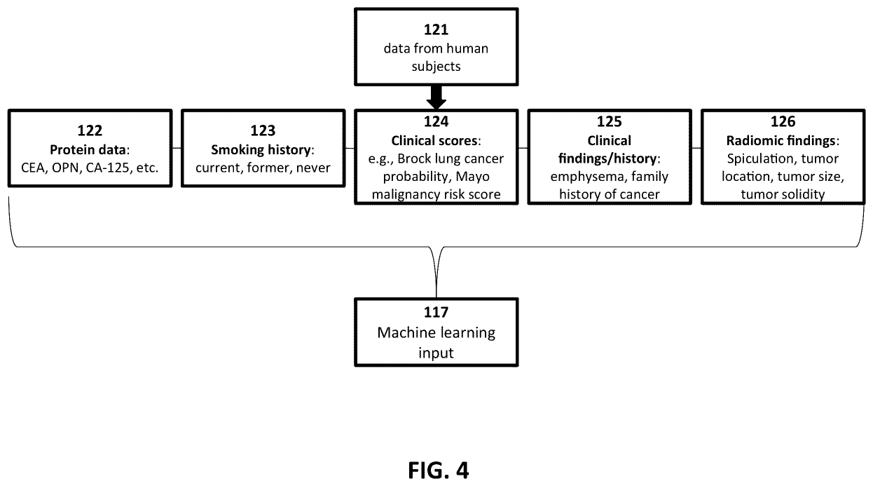

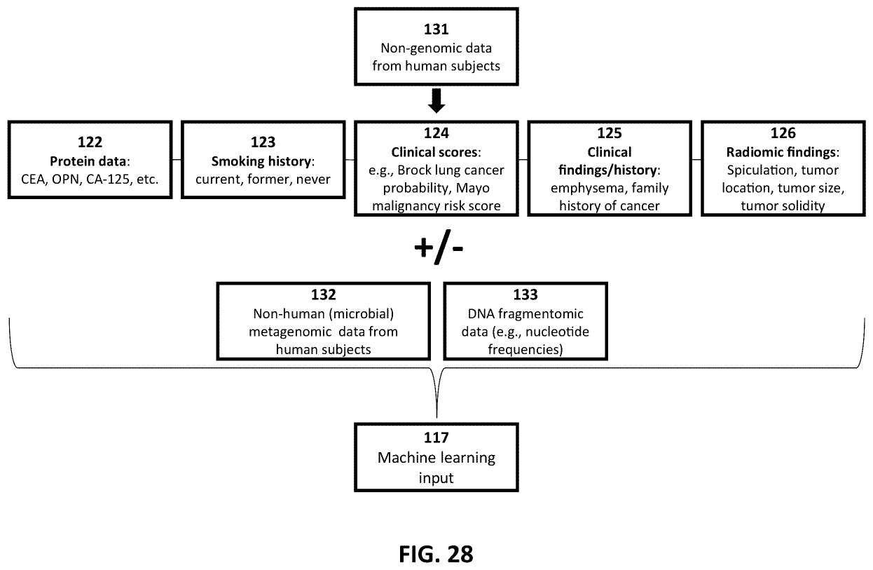

In some embodiments, the data from human subjects 121 may comprise proteomic data 122 in combination non-genomic data 131, e.g., comprising various data features drawn from the subjects' medical histories, as shown in FIG. 4. In some embodiments, the data features may comprise the subjects' smoking histories 123, clinical scores used to assess cancer risk 124, clinical findings and family health histories 125, and data derived from medical imaging 126. These data types may be analyzed alone—in the absence of metagenomic data (FIG. 4)—or may be combined, as shown in FIG. 28, with metagenomic data features (132, 133) to train a predictive model and/or to be analyzed and/or inputted 117 into a previously trained predictive model to determine a diagnostic output, as described elsewhere herein.

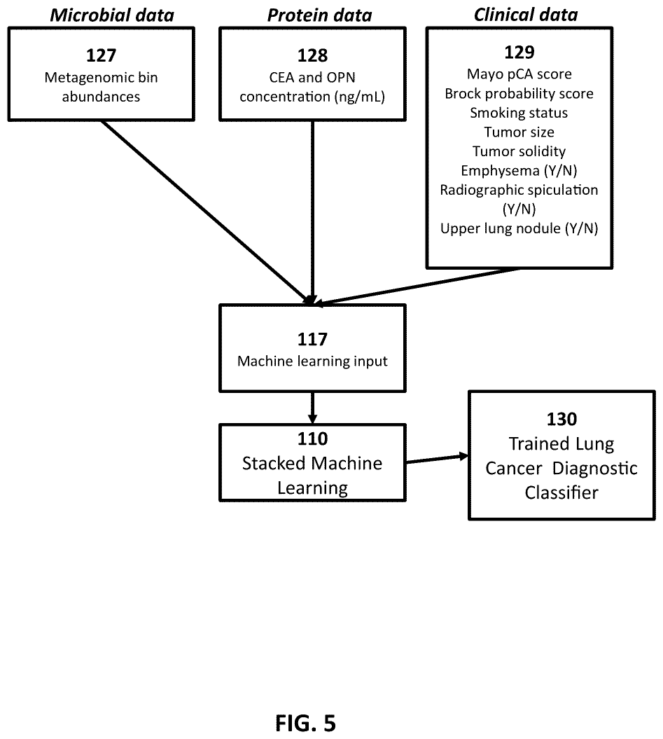

In some embodiments, a trained predictive model (e.g., a trained machine learning classifier) capable of discriminating between malignant cancerous (e.g., lung cancer) nodules and benign cancer nodules (e.g., benign lung cancer nodules) (130, FIG. 5) may be determined and/or obtained as an output of a stacked predictive model 110 using the predictive model input feature tables 117 comprised of metagenomic bin abundances 127 derived from plasma cell-free DNA sequencing data, protein concentrations for the plasma proteins CEA and OPN 128, and/or clinical data obtained from subjects' medical charts 129. In some cases, the clinical data may comprise radiologic data. In some cases, the radiologic data may comprise radiologic images. In some cases, the radiologic images may be generated by X-ray, computed tomography (CT), magnetic resonance imaging (MRI), positron emission tomography (PET), or any combination thereof. In some cases, one or more features of the radiologic data may be determined and utilized to diagnose and/or determine disease of a subject and/or to train one or more predictive model alone in combination with other data types, as described elsewhere herein. In some cases, one or more features of the radiologic data may be analyzed, determined, and/or identified. In some instances, the one or more features of the radiologic data may comprise Brock cancer probability score, cancer lesion diameter, cancer lesion spiculation, cancer lesion solidity, or any combination thereof. In some embodiments, the clinical data features may comprise the Mayo lung cancer probability score, the Brock lung cancer probability score, subject smoking status, or any combination thereof. In some cases, the clinical data features may comprise features determined from medical imaging e.g., lung tumor/nodule size, tumor solidity, evidence of spiculation, lung nodule location, presence of emphysema, or any combination thereof.

### Predictive Models

The methods and/or systems of the present disclosure may utilize and/or access external capabilities of artificial intelligence, predictive models, and/or machine learning techniques to identify one or more microbial features of enriched (e.g., hybridization enriched) biological samples of one or more subjects. In some cases, the microbial features determined from the hybridization enriched biological samples of subjects may predict a cancer and/or a non-cancerous disease of one or more subjects. In some cases, the features may be used to train one or more predictive models (e.g., one or more machine learning algorithms), described elsewhere herein. These features may be used to predict diseases e.g., cancer, non-cancerous diseases, disorders, or any combination thereof. Using such a trained predictive model, health care providers (e.g., physicians) may make informed, accurate risk-based decisions, thereby improving quality of care and monitoring provided to patients with cancer, non-cancerous diseased, disorders, or any combination thereof patient. In some cases, the one or more predictive models may comprise a first predictive model configured to process and/or receive as in input one or more data types, as described elsewhere herein, and a second predictive model configured to receive and/or process an output of the first predictive model, where the second predictive model's output determines and/or diagnoses a disease of the subject. In some cases, at least three predictive models may provide an output to a single predictive model (e.g., a logistic regression model) that may then output a determination, detection, and/or diagnosis of a disease of a subject. In some instances, the at least three predictive models may be trained with one or more data types, as described elsewhere herein. In some cases, the single predictive model receiving input from at least three predictive models may weigh the output of the at least three predictive models with a different weight for each of the models.

The methods and/or systems, described elsewhere herein, may analyze the presence and/or abundance of a microbes (e.g., abundance of microbes of a particular genera and/or taxonomy) of biological sample enriched by hybridization probes where the hybridization probes may bind non-specifically to microbial nucleic acids, as described elsewhere. The presence and/or abundance of microbes may be used to determine one or more microbial features and/or non-microbial features that may predict cancer and/or non-cancerous diseases of one or more subjects. In some cases, the methods and/or systems, described elsewhere herein, may train a predictive model with the one or more microbial features and/or non-microbial features indicative of cancer and/or a non-cancerous disease of a subject. In some cases, the trained predictive model may be used to generate a likelihood (e.g., a prediction) of cancer and/or a non-cancerous disease of one or more subjects that differ from the one or more subjects utilized to train the predictive model. The trained predictive model may comprise an artificial intelligence-based model, such as a machine learning based classifier, configured to process one or more microbial nucleic acid molecule sequencing reads obtained from hybridization enriched biological samples to generate the likelihood of the subject having the disease or disorder. The model may be trained using presence and/or abundance of the microbes of the hybridization enriched biological samples from one or more cohorts of patients, e.g., cancer patients, patients with non-cancerous diseases, patients with no disease and no cancer, cancer patients receiving a treatment for a cancer, patients receiving treatment for a non-cancerous disease, or any combination thereof. In some cases, the predictive model may be trained to provide a treatment prediction to treat a cancer of one or more patients that are not part of the training dataset of the predictive model. Such a predictive model may output a treatment recommendation for the one or more patients that are not part of the training dataset when provided an input of the patient's presence and abundance of one or more microbes of a hybridization enriched biological sample.

The predictive model may comprise one or more predictive models. The model may comprise one or more machine learning algorithms. Machine learning algorithms may comprise a support vector machine (SVM), a naïve Bayes classification, a random forest, a neural network (such as a deep neural network (DNN)), a recurrent neural network (RNN), a deep RNN, a long short-term memory (LSTM) recurrent neural network (RNN), a gated recurrent unit (GRU), a gradient boosting machine, a random forest, or other supervised learning algorithm or unsupervised machine learning, statistical, linear regression, k-nearest neighbors, k-means, decision tree, logistic regression, or any combination thereof model(s). The model(s) may be used for classification or regression. The model may provide an estimate output of an of ensemble of one or more models, comprised of multiple predictive models, and utilize techniques such as gradient boosting, for example in the construction of gradient-boosting decision trees. The model may be trained using one or more training datasets comprising one or more microbial features, patient data e.g., patient medical history, patient's family medical history, patient vitals (e.g., blood pressure, pulse, temperature, oxygen saturation), or any combination thereof.

The predictive model may comprise any number of machine learning algorithms. In some embodiments, the random forest machine learning algorithm may be an ensemble of bagged decision trees. The ensemble may be at least about 1, 2, 3, 4, 5, 10, 20, 30, 40, 50, 60, 70, 80, 90, 100, 120, 140, 160, 180, 200, 250, 500, 1000 or more bagged decision trees. The ensemble may be at most about 1000, 500, 250, 200, 180, 160, 140, 120, 100, 90, 80, 70, 60, 50, 40, 30, 20, 10, 5, 4, 3, 2 or less bagged decision trees. The ensemble may be from about 1 to 1000, 1 to 500, 1 to 200, 1 to 100, or 1 to 10 bagged decision trees.

In some embodiments, the machine learning algorithms may have a variety of parameters. The variety of parameters may be, for example, learning rate, minibatch size, number of epochs to train for, momentum, learning weight decay, or neural network layers etc.

In some embodiments, the learning rate may be between about 0.00001 to 0.1.

In some embodiments, the minibatch size may be at between about 16 to 128.

In some embodiments, the neural network may comprise neural network layers. The neural network may have at least about 2 to 1000 or more neural network layers.

In some embodiments, the number of epochs to train for may be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 500, 1000, 10000, or more.

In some embodiments, the momentum may be at least about 0.1, 0.2, 0.3, 0.4, 0.5, 0.6, 0.7, 0.8, 0.9 or more. In some embodiments, the momentum may be at most about 0.9, 0.8, 0.7, 0.6, 0.5, 0.4, 0.3, 0.2, 0.1, or less.

In some embodiments, learning weight decay may be at least about 0.00001, 0.0001, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, or more. In some embodiments, the learning weight decay may be at most about 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.0001, 0.00001, or less.

In some embodiments, the machine learning algorithm may use a loss function. The loss function may be, for example, regression losses, mean absolute error, mean bias error, hinge loss, Adam optimizer and/or cross entropy.

In some embodiments, the parameters of the machine learning algorithm may be adjusted with the aid of a human and/or computer system.

In some embodiments, the machine learning algorithm may prioritize certain features. The machine learning algorithm may prioritize features that may be more relevant for detecting cancer, non-cancerous disease, disorder, or any combination thereof. The feature may be more relevant for detecting cancer, non-cancerous disease, and/or disorders, if the feature is classified more often than another feature in determining cancer, non-cancerous disease, and/or disorders. In some cases, the features may be prioritized using a weighting system. In some cases, the features may be prioritized on probability statistics based on the frequency and/or quantity of occurrence of the feature. The machine learning algorithm may prioritize features with the aid of a human and/or computer system.

In some cases, the predictive model may prioritize certain features to reduce calculation costs, save processing power, save processing time, increase reliability, and/or decrease random access memory usage, etc.

Training datasets may be generated from, for example, one or more cohorts of patients having common cancer, non-cancerous disease, or disorder diagnosis. Training datasets may comprise one or more microbial features in the form of presence and/or abundance of microbes of an enriched biological sample (e.g., hybridization enriched biological sample) of one or more subjects. Features may comprise a corresponding cancer diagnosis of one or more subjects to microbial features. In some cases, features may comprise patient information such as patient age, patient medical history, other medical conditions, current or past medications, clinical risk scores, and time since the last observation. For example, a set of features collected from a given patient at a given time point may collectively serve as a signature, which may be indicative of a health state or status of the patient at the given time point.

Labels may comprise clinical outcomes such as, for example, a presence, absence, diagnosis, and/or prognosis of cancer, non-cancerous disease, disorder, or a combination thereof, in the subject (e.g., patient). Clinical outcomes may comprise treatment efficacy (e.g., whether a subject is a positive or a negative responder to a cancer and/or disease-based treatment).

Input features may be structured by aggregating the data into bins or alternatively using a one-hot encoding. Inputs may also include feature values or vectors derived from the previously mentioned inputs, such as cross-correlations.

Training datasets may be constructed from presence and/or abundance features of the one or more microbes in the hybridization enriched biological sample or a combination of the presence and/or abundance features of the one or more microbes and the one or more somatic nucleic acid molecule of the enriched biological sample indicative of cancer, non-cancerous diseases, disorders, or any combination thereof.

The model may process the input features to generate output values comprising one or more classifications, one or more predictions, or a combination thereof. For example, such classifications or predictions may include a binary classification of a cancer or no cancer present; presence of a non-cancerous disease; presence of a disorder; or any combination thereof classifications of a subject. In some cases, the one or more predictive models (e.g., machine learning algorithms) may classify subjects between a group of categorical labels (e.g., ‘no cancer, non-cancer disease and/or disorder’, ‘apparent cancer, non-cancer disease and/or disorder’, and ‘likely cancer, non-cancer disease and/or disorder’); a likelihood (e.g., relative likelihood or probability) of developing a particular cancer, non-cancerous disease, and/or disorder; a score indicative of a presence of cancer, non-cancer disease and/or disorder, a ‘risk factor’ for the likelihood of mortality of the patient, a confidence interval for any numeric predictions; or any combination thereof. Various machine learning techniques may be cascaded such that the output of a machine learning technique may also be used as input features to subsequent layers or subsections of the model.

In order to train the model (e.g., by determining weights and correlations of the model) to generate real-time classifications or predictions), the model can be trained using training datasets and/or one or more training features, described elsewhere herein. Such datasets and/or features may be sufficiently large to generate statistically significant classifications or predictions. For example, datasets may comprise databases of data including fungal, viral, archaeal, microbial, bacterial, or any combination thereof microbe presence and/or abundance of one or more subjects' biological samples.

Datasets may be split into subsets (e.g., discrete or overlapping), such as a training dataset, a development dataset, and a test dataset. For example, a dataset may be split into a training dataset comprising 80% of the dataset, a development dataset comprising 10% of the dataset, and a test dataset comprising 10% of the dataset. The training dataset may comprise about 10%, about 20%, about 30%, about 40%, about 50%, about 60%, about 70%, about 80%, or about 90% of the dataset. The development dataset may comprise about 10%, about 20%, about 30%, about 40%, about 50%, about 60%, about 70%, about 80%, or about 90% of the dataset. The test dataset may comprise about 10%, about 20%, about 30%, about 40%, about 50%, about 60%, about 70%, about 80%, or about 90% of the dataset. In some embodiments, leave one out cross validation may be employed. Training sets (e.g., training datasets) may be selected by random sampling of a set of data corresponding to one or more patient cohorts to ensure independence of sampling. Alternatively, training sets (e.g., training datasets) may be selected by proportionate sampling of a set of data corresponding to one or more patient cohorts to ensure independence of sampling.

To improve the accuracy of model predictions and reduce overfitting of the model, the datasets may be augmented to increase the number of samples within the training set. For example, data augmentation may comprise rearranging the order of observations in a training record. To accommodate datasets having missing observations, methods to impute missing data may be used, such as forward-filling, back-filling, linear interpolation, and/or multi-task Gaussian processes. Datasets may be filtered and/or batch corrected to remove or mitigate confounding factors. For example, within a database, a subset of patients may be excluded.

The model may comprise one or more neural networks, such as a neural network, a convolutional neural network (CNN), a deep neural network (DNN), a recurrent neural network (RNN), and/or a deep RNN. The recurrent neural network may comprise units which can be long short-term memory (LSTM) units or gated recurrent units (GRU). For example, the model may comprise an algorithm architecture comprising a neural network with a set of input features, as described elsewhere herein, e.g., microbial features, patient and/or subject vital measurements, patient and/or subject medical history, patient and/or subject demographics, or any combination thereof. Neural network techniques, such as dropout or regularization, may be used during training the model to prevent overfitting. The neural network may comprise a plurality of sub-networks, each of which is configured to generate a classification or prediction of a different type of output information, which may be combined to form an overall output of the neural network. The machine learning model may alternatively utilize statistical or related algorithms including random forest, classification and regression trees, support vector machines, discriminant analyses, regression techniques, any combination thereof, and/or ensemble and gradient-boosted variations thereof.

When the model generates a classification or a prediction of cancer, non-cancerous disease, disorder, or a combination thereof, a notification (e.g., alert or alarm) may be generated and transmitted to a health care provider, such as a physician, nurse, and/or other member of the patient's treatment team within a hospital. Notifications may be transmitted via an automated phone call, a short message service (SMS), multimedia message service (MMS) message, an e-mail, and/or an alert within a dashboard. The notification may comprise output information such as a prediction of cancer, non-cancerous disease, and/or disorder; a likelihood of the predicted cancer, non-cancerous disease and/or disorder; a time until an expected onset of the cancer, non-cancerous disease and/or disorder; a confidence interval of the likelihood or time, a recommended course of treatment for the cancer, non-cancerous disease and/or disorder, or any combination thereof information. In some cases, the time until an expected onset of the cancer may comprise a time of at least 1 year, at least 2 years, or at least 3 years from a detection of a pre-cancerous lesion.

To validate the performance of the model, different performance metrics may be generated. For example, an area under the receiver-operating characteristic curve (AUROC) may be used to determine the diagnostic, prognostic, screening, or any combination thereof capability of the model. For example, the model may use classification thresholds which are adjustable, such that specificity and sensitivity are tunable, and the receiver-operating characteristic curve (ROC) can be used to identify the different operating points corresponding to different values of specificity and sensitivity.

In some cases, such as when datasets are not sufficiently large, cross-validation may be performed to assess the robustness of a model across different training and testing datasets.

To calculate performance metrics such as sensitivity, specificity, accuracy, positive predictive value (PPV), negative predictive value (NPV), area under the precision-recall curve (AUPR), AUROC, or any combination thereof, the following definitions may be used. A “false positive” may refer to an outcome in which a positive outcome or result has been incorrectly or prematurely generated (e.g., before the actual onset of, or without any onset of, the cancer, non-cancerous disease and/or disorder). A “true positive” may refer to an outcome in which positive outcome or result has been correctly generated, when the patient has the cancer, non-cancerous disease and/or disorder (e.g., the patient shows symptoms of the cancer, non-cancerous disease and/or disorder, or the patient's record indicates the cancer, non-cancerous disease and/or disorder). A “false negative” may refer to an outcome in which a negative outcome and/or result has been generated, but the patient has the cancer, non-cancerous disease and/or disorder (e.g., the patient shows symptoms of the cancer, non-cancerous disease and/or disorder, or the patient's record indicates the cancer, non-cancerous disease and/or disorder). A “true negative” may refer to an outcome in which a negative outcome or result has been generated (e.g., before the actual onset of, or without any onset of, the cancer, non-cancerous disease and/or disorder).

The model may be trained until certain pre-determined conditions for accuracy or performance are satisfied, such as having minimum desired values corresponding to diagnostic accuracy measures. For example, the diagnostic accuracy measure may correspond to prediction of a likelihood of occurrence of a cancer, non-cancerous disease and/or disorder in the subject. As another example, the diagnostic accuracy measure may correspond to prediction of a likelihood of deterioration and/or recurrence of a cancer, non-cancerous disease and/or disorder for which the subject has previously been treated. Examples of diagnostic accuracy measures may include sensitivity, specificity, positive predictive value (PPV), negative predictive value (NPV), accuracy, AUPR, and AUROC corresponding to the diagnostic accuracy of detecting or predicting a cancer, non-cancerous disease and/or disorder.

For example, such a pre-determined condition may be that the sensitivity of predicting the cancer, non-cancerous disease and/or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the specificity of predicting the cancer, non-cancerous disease and/or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the positive predictive value (PPV) of predicting the cancer, non-cancerous disease and/or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the negative predictive value (NPV) of predicting the cancer, non-cancerous disease and/or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a pre-determined condition may be that the area under the curve (AUC) of a Receiver Operating Characteristic (ROC) curve (AUROC) of predicting the cancer, non-cancerous disease and/or disorder comprises a value of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

As another example, such a pre-determined condition may be that the area under the precision-recall curve (AUPR) of predicting the cancer, non-cancerous disease and/or disorder comprises a value of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the cancer, non-cancerous disease and/or disorder with a sensitivity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the cancer, non-cancerous disease and/or disorder with a specificity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the cancer, non-cancerous disease and/or disorder with a positive predictive value (PPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the cancer, non-cancerous disease and/or disorder with a negative predictive value (NPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained model may be trained or configured to predict the cancer, non-cancerous disease and/or disorder with an area under the curve (AUC) of a Receiver Operating Characteristic (ROC) curve (AUROC) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained model may be trained or configured to predict the cancer, non-cancerous disease and/or disorder with an area under the precision-recall curve (AUPR) of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

The training data sets may be collected from training subjects (e.g., humans). Each training data set of the training data sets may comprise a corresponding diagnostic status for a given training data of a subject indicating that the subject has either been diagnosed with the disease or have not been diagnosed with disease (e.g., cancer, non-cancerous disease and/or disorder).

In some embodiments, the model is a neural network or a convolutional neural network. See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference.

In some embodiments, independent component analysis (ICA) is used to de-dimensionalize the data, such as that described in Lee, T.-W. (1998): Independent component analysis: Theory and applications, Boston, Mass: Kluwer Academic Publishers, ISBN 0-7923-8261-7, and Hyvarinen, A.; Karhunen, J.; Oja, E. (2001): Independent Component Analysis, New York: Wiley, ISBN 978-0-471-40540-5, which is hereby incorporated by reference in its entirety.

In some embodiments, principal component analysis (PCA) is used to de-dimensionalize the data, such as that described in Jolliffe, I. T. (2002). Principal Component Analysis. Springer Series in Statistics. New York: Springer-Verlag. doi:10.1007/b98835. ISBN 978-0-387-95442-4, which is hereby incorporated by reference in its entirety.

SVMs are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of “kernels,” which automatically realizes a non-linear mapping to a feature space. The hyper-plane found by the SVM in feature space corresponds to a non-linear decision boundary in the input space.

Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests-Random Features,” Technical Report 567, Statistics Department, U.C. Berkeley, September 1999, which is hereby incorporated by reference in its entirety.

Clustering (e.g., unsupervised clustering model algorithms and supervised clustering model algorithms) is described on pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined. Similarity measures are discussed in Section 6.7 of Duda 1973, where it is stated that one way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 218 of Duda 1973. Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda 1973. Criterion functions are discussed in Section 6.8 of Duda 1973. More recently, Duda et al., Pattern Classification, 2nd edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, New Jersey, each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering, where no preconceived notion of what clusters should form when the training set is clustered, are imposed.

Regression models, such as that of the multi-category logit models, are described in Agresti, An Introduction to Categorical Data Analysis, 1996, John Wiley & Sons, Inc., New York, Chapter 8, which is hereby incorporated by reference in its entirety. In some embodiments, the model makes use of a regression model disclosed in Hastie et al., 2001, The Elements of Statistical Learning, Springer-Verlag, New York, which is hereby incorporated by reference in its entirety. In some embodiments, gradient-boosting models are used toward, for example, the classification algorithms described herein; these gradient-boosting models are described in Boehmke, Bradley; Greenwell, Brandon (2019). “Gradient Boosting”. Hands-On Machine Learning with R. Chapman & Hall. pp. 221-245. ISBN 978-1-138-49568-5., which is hereby incorporated by reference in its entirety. In some embodiments, ensemble modeling techniques are used; these ensemble modeling techniques are described in the implementation of classification models herein, and are described in Zhou Zhihua (2012). Ensemble Methods: Foundations and Algorithms. Chapman and Hall/CRC. ISBN 978-1-439-83003-1, which is hereby incorporated by reference in its entirety.

In some embodiments, the machine learning analysis is performed by a device executing one or more programs (e.g., one or more programs stored in the Non-Persistent Memory or in Persistent Memory) including instructions to perform the data analysis. In some embodiments, the data analysis is performed by a system comprising at least one processor (e.g., a processing core) and memory (e.g., one or more programs stored in Non-Persistent Memory or in the Persistent Memory) comprising instructions to perform the data analysis. In some embodiments, the predictive model may be stored on a computer memory and executed by one or more processors of a system, as described elsewhere herein.

### Systems

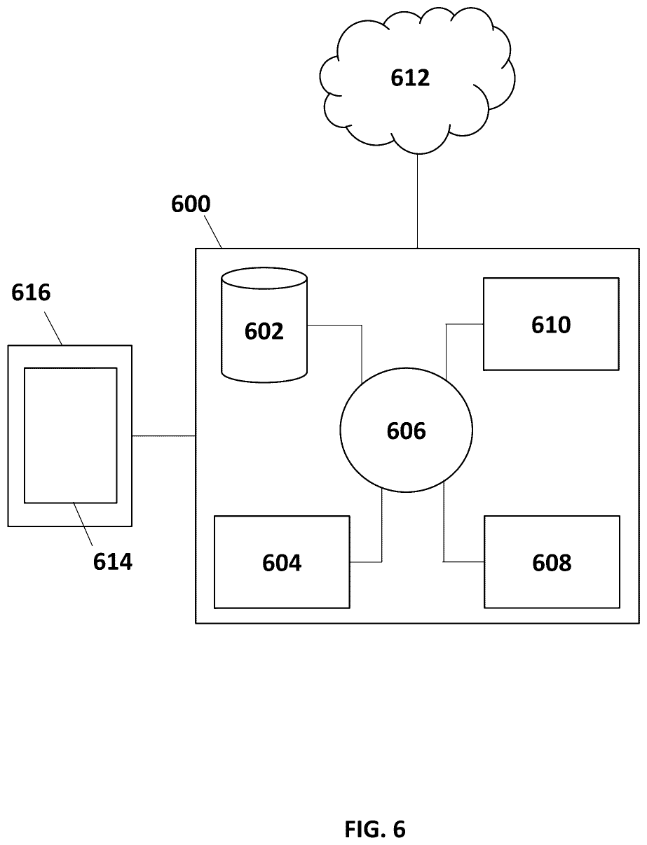

The disclosure, in some embodiments, describes computer systems that may be programmed to implement one or more methods of the disclosure, as described elsewhere herein. FIG. 6 shows a computer system 600 that may be programmed or otherwise configured to predict cancer, non-cancerous disease, or a combination thereof; train a predictive model; generate a recommended therapeutic; or any combination thereof methods, described elsewhere herein. The computer system 600 can be an electronic device of a user or a computer system that is remotely located with respect to the electronic device. The electronic device can be a mobile electronic device.

In some embodiments, the computer system 600 may comprise one or more central processing unit (CPU, also “processor” and “computer processor” herein) 606, which can be a single core or multi core processor, or a plurality of processors for parallel processing. In some embodiments, the computer system 600 may comprise memory and/or memory location 604 (e.g., random-access memory, read-only memory, flash memory), electronic storage unit 602 (e.g., hard disk), communication interface 608 (e.g., network adapter) for communicating with one or more other systems, and/or peripheral devices 610, such as cache, other memory, data storage and/or electronic display adapters. The memory 604, storage unit 602, interface 608 and peripheral devices 610 may be in communication with the CPU 606 through a communication bus (solid lines), such as a motherboard. The storage unit 602 can be a data storage unit (or data repository) for storing data. The computer system 600 can be operatively coupled to a computer network (“network”) 612 with the aid of the communication interface 608. The network 612 can be the Internet, an internet and/or extranet, or an intranet and/or extranet that is in communication with the Internet. The network 612 in some cases may comprise a telecommunication and/or data network. The network 612 can include one or more computer servers, which can enable distributed computing, such as cloud computing. The network 612, in some cases with the aid of the computer system 600, can implement a peer-to-peer network, which may enable devices coupled to the computer system 600 to behave as a client or a server.

The CPU 606 can execute a sequence of machine-readable instructions, e.g., the one or more steps of the methods described elsewhere herein, which can be embodied in a program or software. The instructions may be stored in a memory location, such as the memory 604. The instructions can be directed to the CPU 606, which can subsequently program or otherwise configure the CPU 606 to implement methods of the present disclosure, described elsewhere herein. Examples of operations performed by the CPU 606 can include fetch, decode, execute, and/or writeback.

The CPU 606 can be part of a circuit, such as an integrated circuit. One or more other components of the system 600 can be included in the circuit. In some cases, the circuit may comprise an application specific integrated circuit (ASIC).

The storage unit 602 can store files, such as drivers, libraries, and/or saved programs (e.g., comprising one or more steps of the methods described elsewhere herein). The storage unit 602 can store user data, e.g., classification and/or predictions provided by the one or more models, training user data, test user data, user preferences and user programs, or any combination thereof. The computer system 600, in some cases, can include one or more additional data storage units that are external to the computer system 600, such as located on a remote server that is in communication with the computer system 600 through an intranet or the Internet.

The computer system 600 can communicate with one or more remote computer systems through the network 612. For instance, the computer system 600 can communicate with a remote computer system of a user. Examples of remote computer systems may include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 600 via the network 612.

Methods as described herein can be implemented by way of machine (e.g., one or more processors) executable code stored on an electronic storage location of the computer system 600, such as, for example, on the memory 604 or electronic storage unit 602. The machine executable or machine-readable code can be provided in the form of software. During use, the code can be executed by the one or more processors 606. In some cases, the code can be retrieved from the storage unit 602 and stored on the memory 604 for ready access by the processor 606. In some situations, the electronic storage unit 602 can be precluded, and machine-executable instructions are stored on memory 604.

The code can be pre-compiled and configured for use with a machine having a processer adapted to execute the code or can be compiled during runtime. The code can be supplied in a programming language that can be selected to enable the code to execute in a pre-compiled or as-compiled fashion.

In some embodiments, a system, as described elsewhere herein, may comprise: one or more processors; and a non-transient computer readable storage medium comprising software, wherein the software comprises executable instructions that, as a result of execution, cause the one or more processors of a computer system to: receive one or more nucleic acid molecule sequencing reads of a subject's biological sample, where the subject has a disease, and where the one or more nucleic acid molecule sequencing reads are obtained from one or more nucleic acid molecules enriched by one or more probes exposed to the subject's biological sample; map the one or more nucleic acid molecule sequencing reads to a genome database, thereby identifying one or more non-human sequencing reads of the one or more nucleic acid molecule sequencing reads; and identify one or more microbial features of the one or more non-human sequencing reads to classify the subject's disease.

Aspects of the systems and methods described elsewhere herein, such as the computer system 600, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media may comprise dynamic memory, such as main memory of such a computer platform. Tangible transmission media may comprise coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 600 can include or be in communication with an electronic display 614 that comprises a user interface (UI) 616 for providing, for example, a display for visualization of prediction results may comprise one or more interfaces and/or panels, for training a predictive model, managing and/or manipulating subject and/or patient data, or any combination thereof. Examples of UI's include, without limitation, a graphical user interface (GUI) and web-based user interface.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Although the steps of the methods as described and claimed herein show and/or describe each of the methods or sets of operations in accordance with embodiments, a person of ordinary skill in the art will recognize many variations based on the teaching described herein. The steps may be completed in a different order. Steps may be added or omitted. Some of the steps may comprise sub-steps. Many of the steps may be repeated as often as beneficial. One or more steps may be conducted simultaneously.

One or more of the steps of each of the methods or sets of operations may be performed with circuitry as described herein, for example, one or more of the processor or logic circuitry such as programmable array logic for a field programmable gate array. The circuitry may be programmed to provide one or more of the steps of each of the methods or sets of operations and the program may comprise program instructions stored on a computer readable memory or programmed steps of the logic circuitry such as the programmable array logic or the field programmable gate array, for example.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo”, as used herein, is used to describe an event that takes place in a subject's body.

The term “ex vivo”, as used herein, is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro”, as used herein, is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

The terms “metagenome,” “metagenomes,” and “metagenomic”, as used herein, are used to refer to the sum total of all genomic information represented in a sample, regardless of the species of origin and therefore include both human and non-human genomic information.

The term “metagenomic assembly”, as used herein, is used to refer to the process of reconstructing microbial genomes from metagenomic sequencing data and “metagenomic assemblies” is used to refer to the product of the metagenomic assembly process.

The term “contigs”, as used herein, is used to refer to a non-redundant nucleic acid genome sequence formed by joining, based on sequence overlap, one or more smaller sequences. Contigs, in some cases, may have a length of about a few kilobases (kb)- to about a few hundred kb.

The term “bins”, as used herein, is used to refer to a collection of one or more contigs that have been grouped together due to a likelihood of originating from the same parent genome.

The terms “bin abundance” or “bin abundances”, as used herein, are used to refer to the number of nucleic acid sequencing reads that aligned to one or more bins.

The terms “nodule,” “neoplasm,” and “tumor”, as used herein, are often used interchangeably herein and are intended to denote an abnormal growth of tissue, independent of its malignancy status.

As used herein, the term “about”, as used herein, a number refers to that number plus or minus 10% of that number. The term “about” a range refers to that range minus 10% of its lowest value and plus 10% of its greatest value.

Use of absolute or sequential terms, for example, “will,” “will not,” “shall,” “shall not,” “must,” “must not,” “first,” “initially,” “next,” “subsequently,” “before,” “after,” “lastly,” and “finally,”, as used herein, are not meant to limit scope of the present embodiments disclosed herein but as exemplary.

Any systems, methods, software, compositions, and platforms described herein are modular and not limited to sequential steps. Accordingly, terms such as “first” and “second” do not necessarily imply priority, order of importance, or order of acts.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### EXAMPLES

### Example 1: Early-Stage Detection of Lung Cancer Using Blood and Tissue Metagenomes

Most patients with stage I lung cancer have cell-free tumor DNA fractions of less than 0.1%, precluding sensitive assessments with host-based diagnostics that rely on separating normal from tumor-derived molecules. As described in Example 1, an alternative and orthogonal approach centered on cell-free metagenomes that sidesteps the limitations of host-centric methods was investigated, providing state-of-the-art detection of lung cancers as small as 8 millimeters in diameter. De novo metagenome assembly in 5187 whole-genome sequenced blood and tissue samples were performed, identifying 1562 pan-cancer bins, and demonstrate their diagnostic capacity and cancer type-specificity in 17,079 samples across four independent cohorts. It was also found that amplicon-based (16S rRNA) plasma sequencing approximates the diagnostic performance of shotgun-sequenced metagenomes, suggesting cost-efficient opportunities while validating the approach. Furthermore, since this strategy is independent of and complements host-centric methods, a clinico-proteo-metagenomic diagnostic test, which exceeds the diagnostic performance of PET-CT and clinical risk scores in a blinded validation cohort of 106 stage I cancers and lung diseases of diverse etiologies (Avg±SD=1.91±0.84 cm) was developed and validated. Collectively, the findings described herein, firmly establish the utility of cell-free metagenomes as a generalizable and sensitive strategy for early cancer detection, warranting applications in additional cancer types.

Existing liquid biopsy methods for lung cancer have size-dependent sensitivities that preclude optimal detection of early-stage disease. Orthogonal, cancer-associated, microbial biomarkers can overcome some of these limitations. The study described by Example 1: (i) describes a metagenome-centric lung cancer diagnostic test, (ii) performs metagenomic assemblies in cancer blood and tissues, (iii) evaluates amplicon-based plasma sequencing for cancer detection, and (iv) demonstrates the superiority of this approach to PET-CT in the setting of stage I disease.

Until recently, cancer was predominantly considered a sterile disease of the human genome, and methods to diagnose its presence and/or type have thus relied on the detection of human-centric biomarkers. Although various diagnostic strategies have been developed, ranging from low-plex, PCR-based mutation panels to high-plex assays of millions of DNA fragments, the size of the cancer lesion inherently limits the availability and abundance of cancer-derived molecules in circulation along with the concomitant tests' sensitivities. These challenges can be mitigated, in part, by measuring more biomarkers, deeper sequencing, and/or testing more frequently (e.g., monthly), but there ultimately exists a hard limit imposed by biology on how much cancer-derived material exists in a vial of blood taken during early-stage disease that has precluded widespread diagnostics for those tumors.

Among difficult to detect cancers, lung cancer is the leading cause of cancer-related deaths worldwide and is frequently detected in late-stage disease. Despite recent advancements in liquid biopsies, blood tests capable of detecting stage I lung cancer lesions, particularly those ≤3 centimeters in diameter (i.e., T1 clinical stage), have proven difficult. For example, in this context, the validation cohort of a recent state-of-the-art circulating tumor DNA (ctDNA)-based test, Lung-CLiP, achieved an area under the receiver operating characteristic (AUROC) curve of just 0.69 among stage I cancers versus risk-matched controls. Another recent fragmentomic diagnostic showed an AUROC of 0.76 among stage I cancers in its discovery cohort, although the performance in its validation cohort was not reported other than being approximately 50% sensitive at 80% specificity. Other than PET-CT scans, the only clinically available blood test for early-stage malignancy determination of small nodules (≤3 centimeters) is an integrated clinico-proteomic panel with an AUROC of 0.76 in a validation cohort. These results highlight a need to approach the problem of early lung cancer detection differently.

Based on intracellular, cancer type-specific communities of intratumoral fungi, bacteria, and viruses whose genomic content is detectable in plasma-derived cell-free DNA (15,16), a method is described, as described in example 1 and described elsewhere herein, addressing the problem of early lung cancer detection through microbial biomarkers that are independent of and complementary to host-centric approaches. First, the ability of a parsimonious signature comprising 300 plasma-derived, cancer-associated, fungal, and bacterial biomarkers to diagnose early-stage disease in a low-risk clinical setting of 808 treatment-naive patients, showing state-of-the-art performance and validating the approach with amplicon-based plasma sequencing was evaluated. For the high-risk patient setting, metagenome-assembled bins of cancer-associated microbial DNA, utilizing 5187 whole-genome sequenced blood and tissue samples were developed. These metagenomic bins provided superior diagnostic performance against 222 treatment-naive lung disease samples of diverse etiologies than the 300 known, cancer-associated, microbial biomarkers; moreover, through analyzing their abundances in 17,079 samples from four independent cohorts. The metagenomic bins were found to be broadly cancer type-specific and useful for early-stage lung cancer identification. Finally, using a 454-patient subset of the cohort with matched clinical and imaging metadata a clinico-proteo-metagenomic diagnostic was built and validated providing state-of-the-art diagnostic performance in a 106-patient blinded validation cohort having nodule sizes as small as 6 millimeters. Through this study, as described in Example 1, the utility and generalizability of plasma-derived metagenomes for early-stage cancer detection was demonstrated.

### Subjects

All subject samples were obtained from biobanked, retrospective cohorts from New York University, the University of California, San Francisco, the Miami Cancer Center, University of North Carolina at Chapel Hill, and two commercial sources. Sample collections were performed according to the individual institute's Institutional Review Board approved protocol; all subjects signed written informed consent to provide samples for investigation. Subjects with lung cancer or pulmonary nodules included subjects who had suspicious nodules on chest imaging (low dose computed tomography or chest X-ray) and who underwent diagnostic biopsy or surgical resection. Histopathological diagnosis was performed on all biopsied/resected lung neoplasms and malignant neoplasms were assigned a lung cancer subtype and stage on the basis of histopathology. Disease severity in subjects with sarcoidosis or COPD were scored according to the Scadding staging system and GOLD criteria, respectively. Patients with a prior history of cancer or recent (within the last 2 months) antibiotic use were excluded from the study.

454 (44%, 342 lung cancer, 112 non-cancer) samples were originally collected at the NYU Lung Cancer Biomarker Center from pulmonary nodule-bearing subjects who underwent diagnostic biopsy or tissue resection in the period between 2006 and 2021. 89 subject plasma samples were obtained from the SubPopulations and InteRmediate Outcome Measures. In COPD Study, SPIROMICS; all 89 subjects were current smokers, 47 were diagnosed with GOLD Stage II (mild) COPD and the remaining 42 had GOLD Stage III (severe) COPD. 25 subject samples with pulmonary sarcoidosis were obtained from the UCSF Sarcoidosis Research Program; 12/25 had Scadding Stage 2 sarcoidosis and 13/25 had Scadding Stage 4. Of the 402 cancer samples, the two major histological subtypes of lung cancer—lung adenocarcinoma (LUAD) and squamous cell carcinoma (LUSC) comprised 57.2% (230) and 29.8% of the cancer samples, respectively. The number (percentage) of cancers represented by pathological stage is as follows: Stage I, 131 (32.59%); Stage II, 110 (27.36%); Stage III, 98 (24.38%); Stage IV, 57 (14.18%); unknown, 6 (1.49%). Smoking status (current/former/never) was known for 708 (68.73%) of the cohort subjects. All CODICES cohort samples (1030) had shotgun metagenomic sequencing and a subset of 335 samples (142 lung cancer, 97 lung disease, and 96 healthy) had targeted 16S rRNA gene sequencing. All cancer plasma samples were obtained from treatment naive patients and all disease state samples were age (±5 years) and gender matched with healthy donor plasma samples from two commercial sources.

### Sample Processing and Sequencing Library Preparation

Total circulating DNA was extracted from a volume of 400 μL plasma from each sample using the QIAamp Circulating Nucleic Acid Kit (QIAGEN 55114) according to the manufacturer's instructions and purified with Agencourt AMPure XP beads (Beckman Coulter). Sequencing libraries were prepared from the cfDNA using the KAPA HyperPlus Kit (Roche Diagnostics) and unique dual-indexed primers. The final libraries were analyzed using the Agilent 4200 TapeStation System (High Sensitivity DNA Kit) and quantified by qPCR using the NEBNext Library Quant Kit for Illumina (New England Biolabs). Paired-end 2×150-bp sequencing was performed on a NovaSeq 6000 instrument, S4 flow cell (Illumina).

### 16S Sequencing

The circulating V6 region of the 16S ribosomal RNA was targeted with the primers 967F (0.3 μM, 5′-CNACGCGAAGAACCTTANC-3′) and 1064R (0.3 μM, 5′-CGACRRCCATGCANCACCT-3′), and initially amplified by PCR using 2×KAPA HiFi HotStart ReadyMix (Kapa Biosystems, Boston, MA, USA) and 10 ng of input DNA. The cycling program was set as follows: 5 minutes at 95° C., 20 cycles of 20 seconds at 98° C., 15 seconds at 52° C., 15 seconds at 72° C., and a final extension of 5 minutes at 72° C. These PCR products were subjected to two subsequent rounds of 10-cycles PCR following the approach described by Glenn et al. (39) to introduce universal adapters suitable for Illumina sequencing platforms and unique sample-identifying dual indices. PCR conditions were the same as before except for the annealing temperature, brought to 60° C., and the primers concentration, increased to 0.5 μM. All the PCR products were purified using AMPure XP Beads (Beckman Coulter #A63881) following manufacturer's instructions. Final libraries were quantified using Qubit DNA assay (Thermo Fisher Scientific) and via qPCR using the NEBNext Library Quant Kit for Illumina (New England Biolabs), and their quality checked on an Agilent 4200 TapeStation System. The sequencing analysis was performed on an Illumina MiSeq instrument using the Reagent Kit v2 (Illumina, #MS-102-2002).

### Shotgun Metagenomic Sequencing Analysis

Reads were quality filtered using fastp to remove adapter sequences and low-quality reads. Reads aligning to the human genome were separated from non-human reads by alignment to the complete human reference genome T2T-CHM13 (v2.0) using Bowtie2 with the sensitive parameter set. Reads not aligning to the human genome were de-replicated using vsearch. Non-human reads were aligned to the RefSeq database release 206 using Bowtie2 with the sensitive parameter set. Abundances of each microbe were totaled from the alignments and used for downstream Machine Learning analyses.

### Plasma Proteome Analysis

The Bio-Plex 200 platform (Bio-Rad, Hercules, CA) was used to assess levels of target proteins in human plasma samples. Briefly, plasma samples were centrifuged, diluted 1:2, and subjected to Milliplex bead-based immunoassays (Millipore Sigma, Burlington, MA), following manufacturer's protocol. The Milliplex HCCBP1MAG-58K (Millipore Sigma, Burlington, MA) panel was used to detect the following protein analytes: cancer antigen 15-3 (CA15-3), cancer antigen 19-9 (CA19-9), carcinoembryonic antigen (CEA), cancer antigen 125 (CA 125), interleukin-8 (IL-8), prolactin (PRL), cytokeratin 19 fragment (CYFRA 21-1), and osteopontin (OPN). Concentration of each protein analyte was determined using 5 parameter logistic curve fit available in the Bio-Plex Manager 6.2 software (Bio-Rad, Hercules, CA) and protein standards provided with the Milliplex assay.

### TCGA and Plasma Cell-Free Microbial DNA Metagenomic Assembly, Binning, and Analysis

First, preprocessed (i.e., trimmed, quality controlled, and human read filtered) whole genome sequenced (WGS) samples were coassembled by cancer and sample type. Coassembly by sample and cancer type was motivated by past findings that microbes are distributed differently across cancer types and that most individual samples had insufficient read depths to assemble microbial genomes. Co-assemblies were performed through metaSPAdes (v. 3.13.1) with an allocated memory of 1 TB RAM limit, across ten threads, and k-mer sizes 21, 33, 55, 77, 99, and 127. The resulting contigs from each co-assembly were filtered for a length of greater than 1500 and separated as being either prokaryotic or eukaryotic in origin with EukRep (v. 0.6.6). Each set of prokaryotic or eukaryotic contigs were binned using Vamb (v. 3.0.3) with default parameters on contig abundance profiles estimated independently per sample through the MetaBAT2 (v. 2.12.1) jgi_summarize_bam_contig_depths function. Abundance profiles for each sample were estimated by mapping reads against binned contigs using the Salmon (v. 0.13.1) quant function with the—meta flag enabled. Quality metrics for the resulting prokaryotic refined bin sets were calculated using CheckM (v. 1.0.13). All prokaryotic bins were filtered based on CheckM statistics completeness greater than 10 percent and contamination less than 5 percent.

### Microbial Diversity Analysis

Alpha and beta diversity was calculated using Qiime2 on microbial abundance tables of Rep206 species filtered to the overlap with the WIS dataset. For alpha diversity, samples were first rarefied to the minimum number that would retain the upper quartile of samples. Statistical differences between groups were calculated using Mann-Whitney-Wilcoxon test two-sided test. Beta diversity distances and ordination was calculated using DEICODE's Robust Aitchison PCA metric. Statistical differences between groups based on beta diversity was determined using a PERMANOVA test.

### 16S Sequencing Analysis

Raw 16S reads were quality filtered using fastp to remove adapter sequences and low-quality reads. Unique 16S sequences were identified and differentiated from sequencing noise using Deblur within Qiime 2. As a secondary method, sequences were also clustered at 90% OTUs using vsearch. Deblur sub-operational-taxonomic-units (sOTU) and 90% OTUs were taxonomically identified using a trained Greengenes 13_8 99% OTU classifier and the classify-sklearn method in Qiime 2. The resulting feature tables were used for downstream machine learning analyses.

PICRUSt2 was also run on the 90% OTU sequences. First, the 90% OTU table was filtered to a minimum frequency of 10 sequences and minimum prevalence of 10 samples. Then PICRUSt2 was run on the filtered table with the output data types of KEGG Orthologs, Enzymes, and Pathways. These data tables were used for downstream machine learning analysis.

### Genome Coverage Analysis

Genomic coverage of each genome in Rep206 was calculated. Briefly, for each microbial genome, the total number of unique covered bases in the genome was calculated by aggregating the alignments from all plasma samples in the dataset. The same process was applied to all sequencing blank samples. The portion of covered bases over the total bases in the genome was then calculated for each Rep206 genome in plasma samples and blank samples.

### Stacked Machine Learning Approach

Different ML architectures can provide different performances on the same feature set. It was found that the observation was pertinent when attempting to concatenate multi-omic, multi-species (host and microbial) data together, particularly when concatenating categorical data (e.g., smoker status) and quantitative data (e.g., metagenomic abundances) on the same samples. In general, logistic regression-like classifiers performed better on categorical data and protein abundances but worse on metagenomic data. This suggested the need to either heavily optimize the hyperparameters of a given singular ML model to reach a compromise in model performance on multiple kinds of data, or to identify a different mechanism by multiple models could be integrated.

Drawing inspiration from a recent multi-omic model for predicting breast cancer therapy response (PMID: 34875674) and modem convolutional neural networks (PMID: 31631918), stacked ensemble modeling was implemented to integrate predictions from multiple models, each of which would ideally ‘pick up’ different aspects or distributions in the data. Combinations using two or more of the following models was explored: random forests, gradient boosting machines, logistic regression, elastic net, support vector machines (linear, radial, and radialSigma). Internal testing (data not shown) revealed that optimal performance was often reached using three models in tandem: random forests, elastic net, and gradient boosting machines. Thus, these three base algorithms were used for the ensemble modeling, followed by a meta learner comprising a logistic regression to weigh the probability outputs of the base algorithms while calculating a final prediction based on the weighting. To ensure that the base algorithms were aligned and simultaneously providing predictions on the same holdout folds, standardized 10-fold cross-validation indices were predefined and fed into the ensemble modeling. The caretEnsemble (https://github.com/zachmayer/caretEnsemble) R package was employed to perform the model tuning, CV-fold synchronization, hyperparameter tuning of the base algorithms, and weight tuning of the meta-learner.

For the base algorithms, the number of hyperparameter combinations was modified using caretEnsemble's “tuneLength” argument in the caretListo function and was set equal to 20. Since this value denotes the maximum number of iterations to try for each hyperparameter in the model, which may have several hyperparameters, the total number of hyperparameters evaluated is a combinatorial and additive value. For example, the random forests base algorithm has a single tunable hyperparameter, ‘mtry’, which means up to 20 mtry values would be attempted, as follows: {2, 3, 4, 7, 10, 16, 25, 39, 60, 91, 140, 215, 328, 503, 770, 1178, 1802, 2757, 4219, 6454}. Distinctly, gradient boosting machines have two tunable hyperparameters (‘n.trees’ and ‘interaction.depth’), meaning that up to 20*20 values for each would be attempted including combinations, or up to 400 total. These hyperparameters ranged as follows: n.trees, 50 to 1000 by 50; interaction.depth, 1 to 20 by 1. Similarly, elastic net models have two tunable hyperparameters (‘alpha’, ‘lambda’), leading to 20*20 combinations. The automatically generated possible alpha parameters included: {0.1, 0.147368421052632, 0.194736842105263, 0.242105263157895, 0.289473684210526, 0.336842105263158, 0.384210526315789, 0.431578947368421, 0.478947368421053, 0.526315789473684, 0.573684210526316, 0.621052631578947, 0.668421052631579, 0.71578947368421, 0.763157894736842, 0.810526315789474, 0.857894736842105, 0.905263157894737, 0.952631578947368, 1}. The automatically generated possible lambda parameters included: {0.00662229466399123, 0.00824606200985824, 0.0102679723752198, 0.0127856492677635, 0.0159206531946638, 0.0198243509450728, 0.0246852240035688, 0.0307379689652752, 0.0382748293462366, 0.0476597059206647, 0.0593457268717406, 0.0738971260921729, 0.0920164859802709, 0.114578660090195, 0.142673013517157, 0.177656020501926, 0.221216758814626, 0.275458463170505, 0.343000075305506, 0.427102693834312}. In cases where the number of features was less than the hyperparameters permitted, then those respective hyperparameters were excluded from the grid search. Unless the feature set was subsetted (e.g., PET-CT SUVs only), the hyperparameter search for the base algorithms occurred step-wise (within each model) across up to 820 distinct combinations.

For model comparisons with minimal feature sets, such as only CEA or OPN or PET-CT SUVs, stacked ML architecture was used and matched CV-folds to ensure comparable performance values. The only item that varied was that, for singular features, it was not possible to include elastic net in the stacked ML since it cannot regularize a single feature; as a substitute in these situations, elastic net for a plain logistic regression was swapped and used alongside the random forest and gradient boosting machine base algorithms and meta-learner.

To (i) ensure approximately equal representation of samples across sequencing runs and diagnoses within individual CV-folds, (ii) ensure that CV-folds were matched during base algorithm learning, and (iii) provide reproducibility and comparability across feature sets, per-CV-fold indices were calculated and saved within the metadata for each comparison type after subsetting but prior to stacked ML. For example, for the lung cancer versus healthy comparisons using metagenomic data (FIGS. 7C-1 and 7C-2), the following was done: (a) starting with metadata for the whole CODICES cohort, samples were subset to the 808 healthy or cancer-bearing subjects; (b) using the data subset, predefined 10-fold CV indices were calculated based on stratified random sampling across sequencing runs and diagnoses in the metadata to ensure equal representation, and these indices were saved as a new metadata column; (c) during stacked ML, the predefined CV-folds were used for the base algorithms and meta-learner. Furthermore, since these CV-folds were decided on a sample metadata basis, it meant that all feature sets were trained and evaluated on the same CV-folds. Thus, the different feature set performances observed (FIGS. 7C-1 and 7C-2) are directly comparable to one another, as well as the AUROC confidence intervals calculated by using performance on the individual CV-folds.

For metagenomic data normalization, unsupervised batch-corrected metagenomic count data (see above) were transformed using the center log-ratio transform prior to stacked ML. This was done on using the respective subset just prior to stacked ML, meaning that, for example, it would occur on the 808 healthy and lung cancer samples during respective stacked ML (FIGS. 7C-1 and 7C-2) rather than on the full CODICES cohort (n=1030).

Since protein concentrations were already corrected on a plate-by-plate basis using multidimensional normalization (see above) (PMID: 27570895), they were not transformed further. If included, normalized CEA and OPN values were simply concatenated as features to the larger feature table prior to stacked ML.

For clinical metadata normalization, distinct approaches were applied depending on the class of data. For numeric data (e.g., tumor sizes in centimeters), the available data was coerced to be numeric and any resultant “NA” values were assigned “0” (i.e., in a logistic regression, they would ‘drop out’, since any weighted coefficient multiplied by 0 is 0). For categorical data, the features were factored. For boolean data, the data were coerced to logicals (i.e., binary data), except in the case where data were incomplete, in which a third category (“UNKNOWN”) was added. Categorical and boolean columns, post-normalization, were then converted into dummy variable columns using caret's dummyVars( ) function. For example, a single column with categorical data having three levels (“levelA”, “levelB”, “levelC”) would be converted into a three-column data frame of binary (1 or 0) entries, with column names of “levelA”, “levelB”, and “levelC”. When multiple metadata variables were used, such as a numerical variable with a categorical variable, the resultant normalized columns with dummy variables were concatenated together. Of note, the log-odds and probabilities of the Mayo clinical risk score for nodule malignancy (“pCA”) were calculated using existing clinical metadata variables in our data, when possible, as follows: {logOdds=0.0391*age+0.1274*tumor_size_mm+0.7917*smoker_binary+0.7838*upper_lung_nodule_binary+1.0407*spiculation_binary−6.8272}. Of note, all of the CODICES cohort samples did not have a prior history of cancer, so this was excluded from the logOdds calculation; additionally, since the diagnostic model is designed to be independent but complementary to PET-CT imaging, PET-CT, which is optional in the pCA calculation, was also excluded from the logOdds equation. The logOdds were then converted into pCA probabilities, as follows: {pCA_probability=100*exp(logOdds)/(1+exp(logOdds))}.

As an important note, attention was provided to and mitigated situations in which missing data artificially inflated ML performance. This issue was identified when originally including ‘smoker status’ in the low-risk clinical setting models (FIGS. 7C-1 and 7C-2); although including it led to much higher AUROCs between lung cancer-bearing subjects and healthy controls, inspection of the trained feature importance showed that “UNKNOWN” smokers was one of the highest-ranking variables. Further inspection of the metadata then revealed the issue, wherein most patients with lung cancer had known smoking statuses (394/401 with valid entries) but healthy subjects did not (115/408 with valid entries). Hence, the missing smoking status, which was transformed into “UNKNOWN” during metadata normalization was contributing to artificial performance gains. This is, in part, why for healthy versus lung cancer comparisons (FIGS. 7C-1 and 7C-2) and the initial lung disease versus lung cancer comparisons (FIGS. 8G-1 and 8G-2), only metagenomic and/or proteomic data was used, which was available for 100% (1030/1030) or 99.22% (1022/1030), respectively, of the total CODICES cohort. Furthermore, the subsequent clinico-proteo-metagenomic models that utilized clinical metadata were trained and evaluated only using a subset of the CODICES cohort (n=454) where most samples contained that information (e.g., tumor sizes were available for 431/454 samples).

To calculate ROC curves, the predictions on the holdout folds (using the tuned stacked ML model) were concatenated to produce a single prediction set with the same number of original samples. For example, data for 808 samples were input into the stacked ML for lung cancer versus healthy comparisons (FIGS. 7C-1 and 7C-2), and the predictions list had 808 rows associated with it, one for every sample. Of note, this table also included information about which samples belonged to which holdout folds, enabling calculation of the AUROC on each CV-fold, which were then aggregated to estimate the 95% confidence interval of performance. Additionally, the full-length, concatenated prediction list was subset to lung cancers of individual clinical stages and histological subtypes, plus their control samples (i.e., healthy controls or lung disease), followed by regeneration of the ROC curve (e.g., FIGS. 7C-1 and 7C-2). This process has been done elsewhere by Mathios et al. (7). In these cases, the CV-fold information after the sample subsetting was used to estimate the confidence intervals of AUROC performance in each stage and histological subtype comparison. To be clear, this means that in FIGS. 7C-1 and 7C-2, for a given feature set, a single stacked model was built and evaluated as a single diagnostic test.

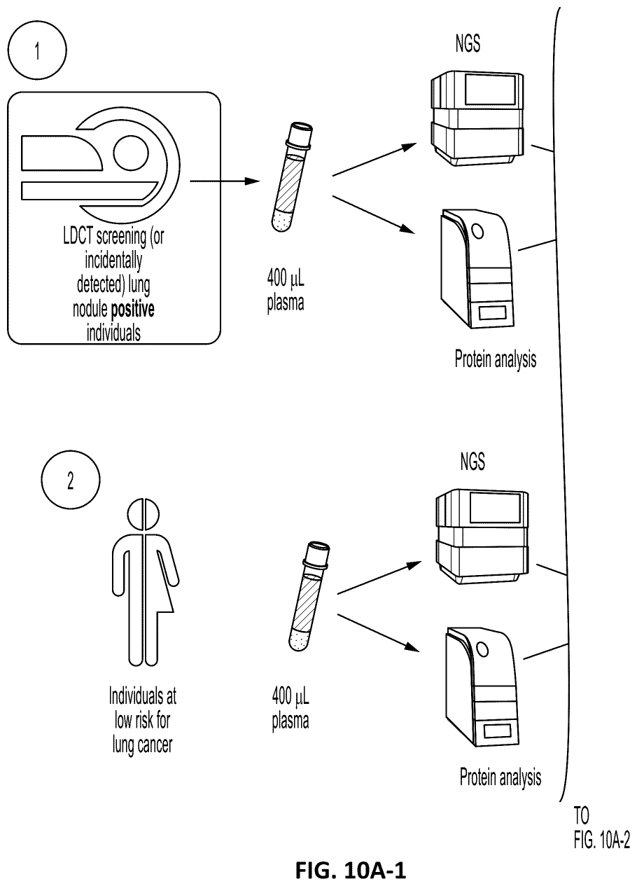

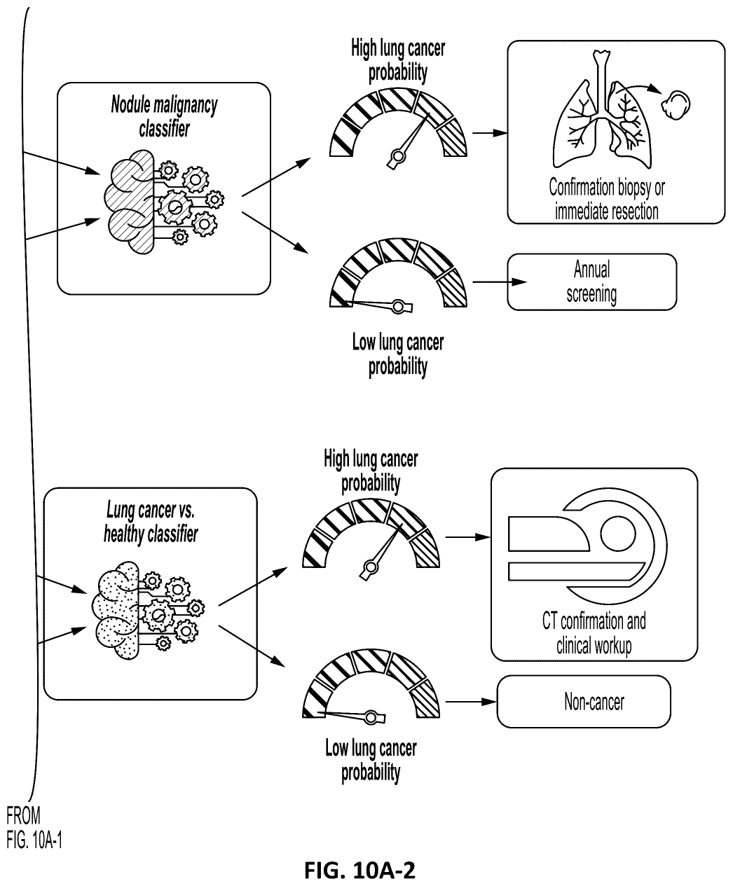

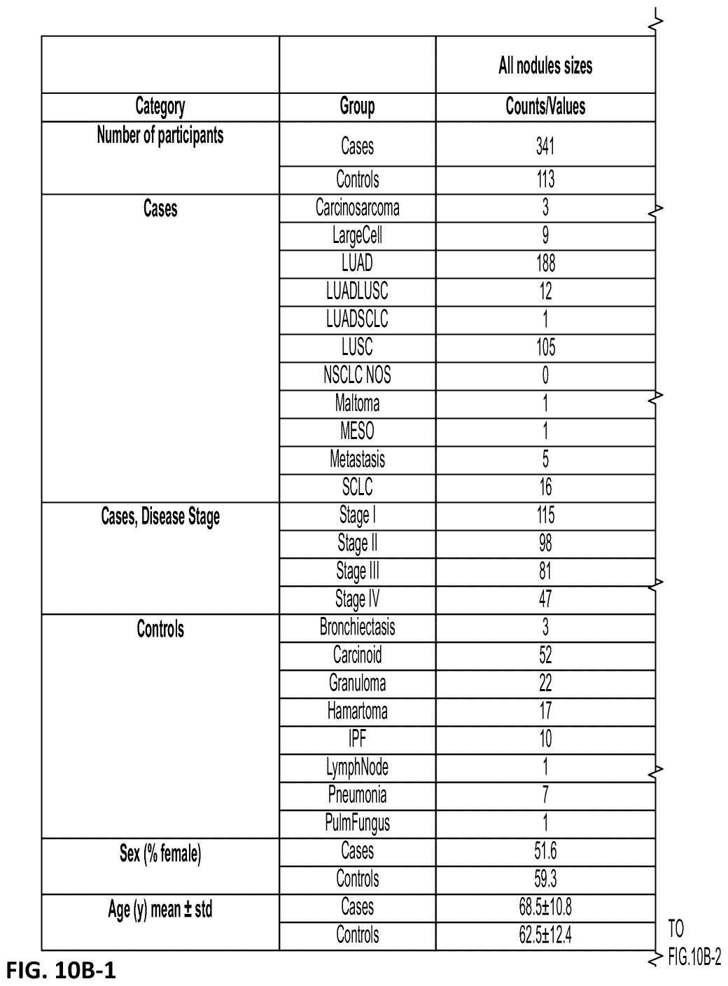

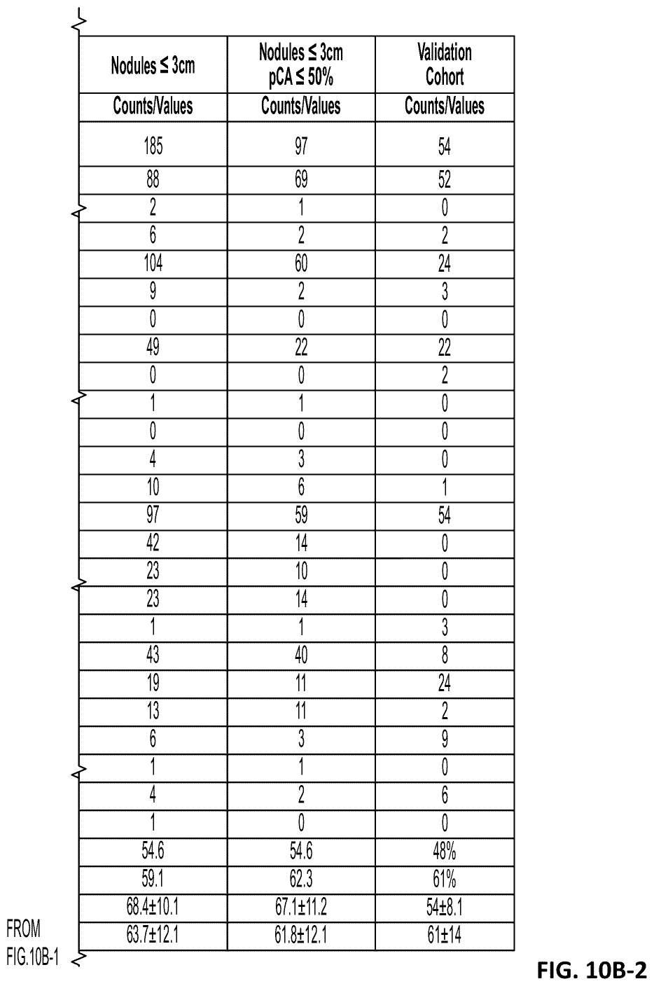

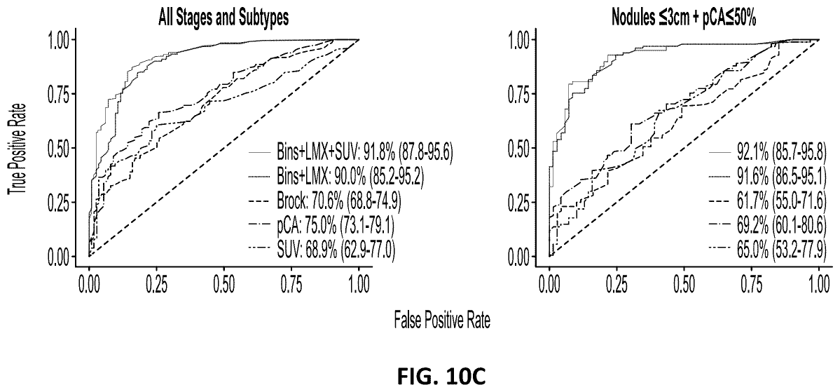

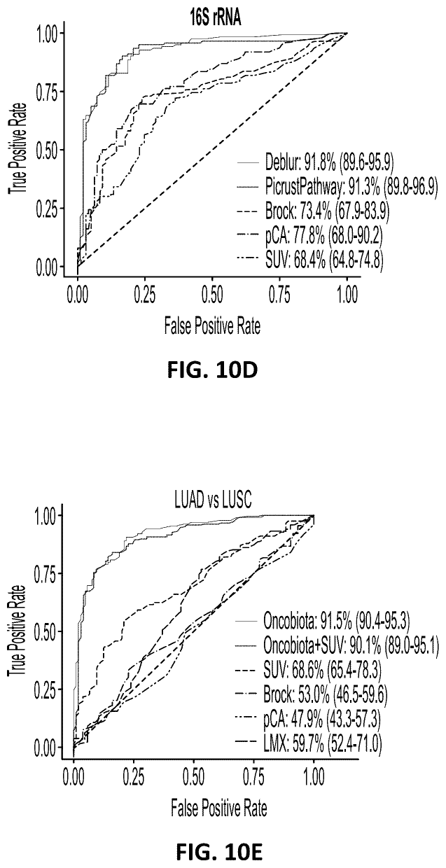

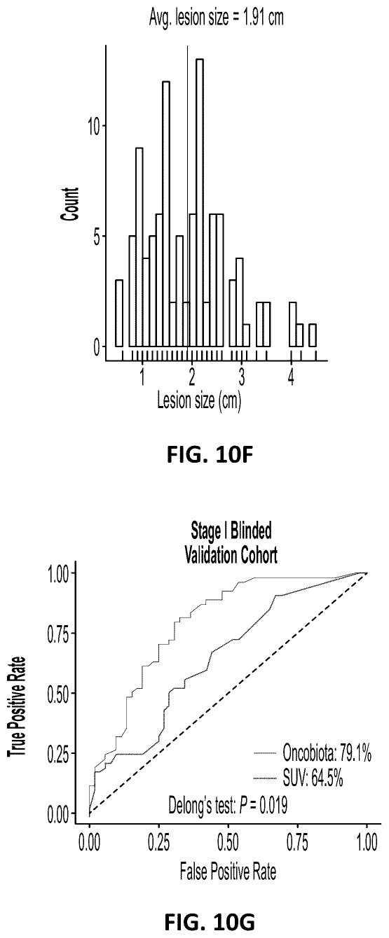

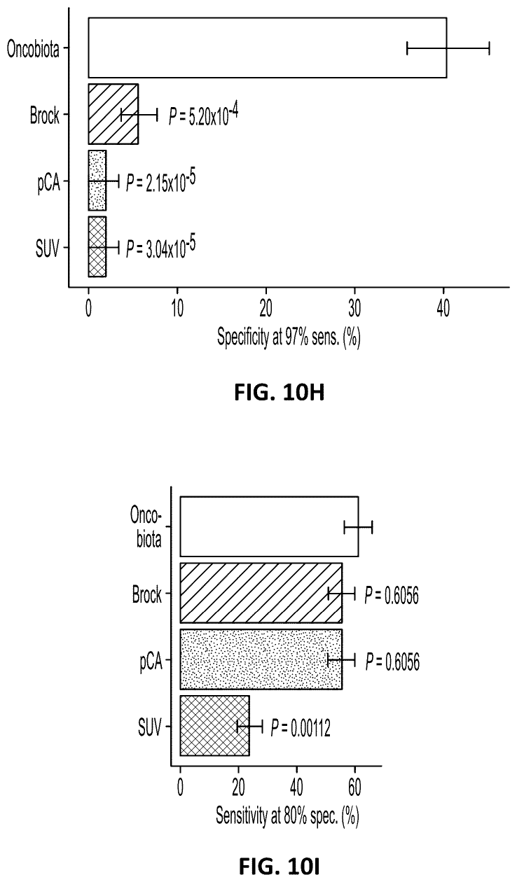

For the 16S rRNA cohort, which was a subset of the CODICES cohort processed independently for amplicon-based sequencing, the above stacked ML architecture was used identically by swapping out the microbial feature table for 16S rRNA taxonomic or PICRUSt2-predicted functional pathway abundances. Moreover, since CEA, OPN, and clinical metadata were available on the overlapping patients in the CODICES cohort, it permitted clinico-proteo-metagenomic evaluation with the amplicon-derived abundances (FIG. 10D).

Another way to integrate the predictions of multiple models is ‘multimodal’ learning, in which multiple classifiers are independently developed on different features of the same samples, and predictions from those classifiers are integrated using a meta-learner. This is distinct from the stacked ensemble learning used, wherein a single feature table is being evaluated by multiple classifiers, followed by a meta-learner. Although such ‘multimodal’ learning approaches (data not shown) have been evaluated, they were not found to perform better than stacked ensemble models while also finding them more complicated to optimize.

### CODICES Cohort: Shotgun Metagenomic Alignments, Decontamination, and Normalization

Reads were quality filtered using fastp to remove adapter sequences and low-quality reads. Reads aligning to the human genome were separated from non-human reads by alignment to the human reference genome GRCh38 using Bowtie2 with the sensitive parameter set followed by alignment to GRCh38 using SNAP. Reads not aligning to the human genome were de-replicated using vsearch. Non-human reads were aligned to the RefSeq database release 206 (“rep206”) using Bowtie2 with the sensitive parameter set. Abundances of each microbe were totaled from the alignments and used for downstream analyses.

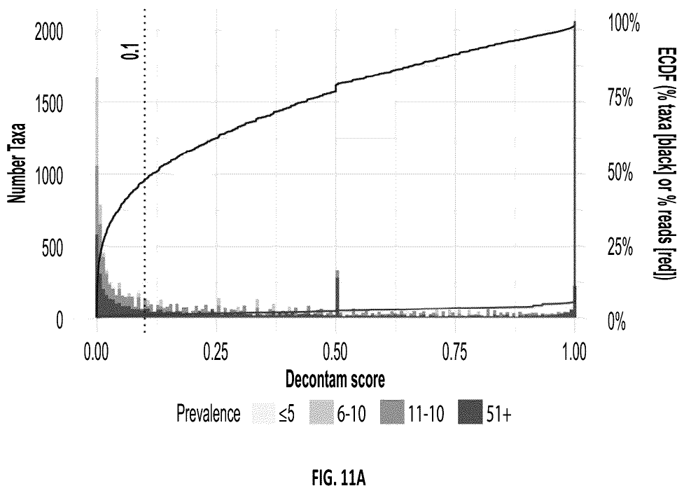

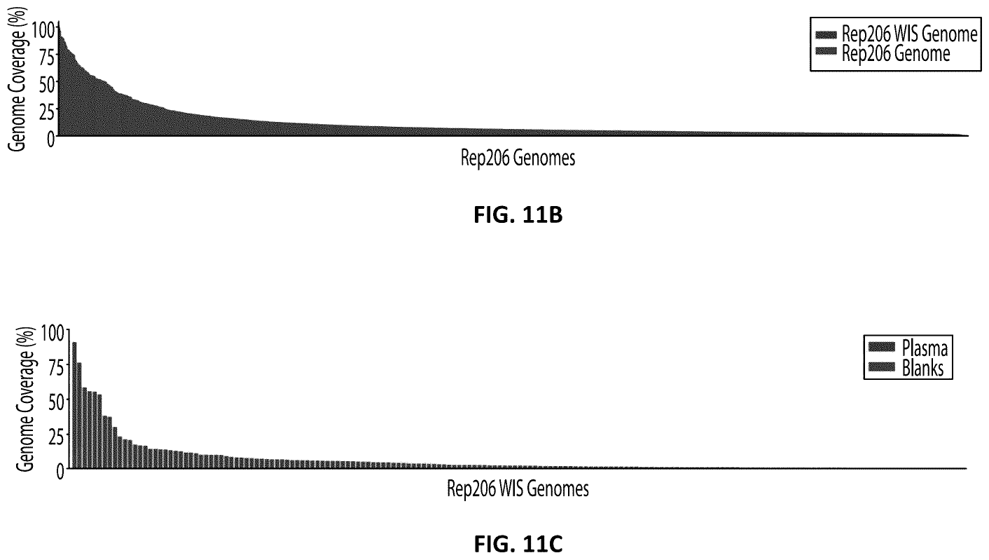

For in silico decontamination, decontam was applied in prevalence mode using all 98 extraction blanks that were processed in parallel with the CODICES cohort plasma samples, using default parameters. After reviewing the histogram of decontam's calculated prevalence p-statistics (FIG. 11A), the default cutoff of P*=0.1 was kept. it was noted that in prevalence mode, the p-statistic is set equal to a p-value from the chi-square or Fisher's exact tests, although it is not directly treated as a p-value to make decisions about particular taxa. It was also noted that prevalence-based decontamination remains valid in extremely low biomass environments. In total, 5630 taxa in the raw rep206 data in the CODICES cohort were flagged as putative contaminants and 6455 taxa were retained (53.41% of total); the 5630 removed taxa accounted for 0.88% of the total read counts.

For feature set intersections with Tsay et al. (“Zebra: Static and Dynamic Genome Cover Thresholds with Overlapping References.” mSystems 2022; e0075822; hereinafter “Tsay”), 16S rRNA results were shared by the respective first author and used to identify overlapping microbes in the rep206 feature set at the genus level. Specifically, since the shotgun metagenomics data for the CODICES cohort utilized species elsewhere (e.g., “Decontaminated” and “WIS” feature sets), all species that had corresponding genus-level overlap with the 16S rRNA data collected from bronchoalveolar lavage samples by Tsay was retained.

For feature set intersection with the Weizmann Institute of Science (WIS)'s catalog of cancer-associated bacteria and fungi, the first author of Narunsky-Haziza et al (“Pan-cancer analyses reveal cancer type-specific fungal ecologies and bacteriome interactions. Cell” hereinafter “Narunsky”). shared a “hit” list of bacteria and fungi across all tissue samples (tumors, NATs, or true normals [breast only]) from the two studies. This “hit” list was filtered to microbes that had species-level results using their multi-region amplicon sequencing approach for bacteria or ITS2 sequencing for fungi, which in turn were intersected with the CODICES cohort's rep206 data, with 300 remaining overlapping features.

Due to their low-biomass nature, investigations of cancer-associated bacteria have previously shown batch effects that often need to be accounted for prior to downstream analyses. Past analyses in TCGA showed this especially to be particularly necessary when comparing data across sequencing centers, sequencing platforms, and experimental strategies (WGS vs. RNA-Seq). In the CODICES cohort, it was also observed that sequencing run-to-run effects in the raw data required correction prior to downstream analyses to ensure that results were not explained by sequencing run-to-run variation (despite additional sample type randomization across sequencing runs that mitigates this effect). Moreover, although batch effect correction using a combination of Voom and SNM in TCGA cancer microbiome data were previously addressed, with useful results, the Voom-SNM strategy is inherently a supervised one that is difficult to use in a diagnostic approach. More specifically, biological information cannot be used to supervise the batch correction if the goal of the test is to obtain such information, unless the supervising information is readily obtainable in some other manner (e.g., using age and/or sex as biological variables). After evaluating various methods (data not shown), it was found that ComBat-Seq, which is designed to work with discrete counts based on a negative binomial model, to completely remove the sequencing run effect on the CODICES cohort's raw rep206 data when applied in an unsupervised manner across sequencing runs. Specifically, ComBat-Seq (in R's sva package v. 3.35.2) was run using default parameters with a single vector indicating which sequencing run each sample belonged to (the “batch” variable) without any other additional information (i.e., unsupervised correction). One additional advantage of the ComBat-Seq approach is that the input and output are both discrete counts, unlike Voom-SNM, which involves log-transformations and concomitant pseudocounts to enable those log-transformations. This means that the ComBat-Seq-corrected counts are in the same units as the original counts and can be directly used for downstream analyses that require discrete count data inputs. For the CODICES cohort data subsets (FIGS. 7C-1 and 7C-2), the taxonomic features first (e.g., down to the 300 WIS-overlapping features, or the 6530 Tsay overlapping features) was pruned prior to applying the unsupervised ComBat-Seq normalization on the entire 1030-sample dataset for that feature set. Metagenomic bin abundances were treated the same, performing unsupervised ComBat-Seq on the whole CODICES cohort, again with default parameters and a single “batch” vector denoting the sequencing run of each sample.

All downstream analyses then used a single, unsupervised batch-corrected, normalized dataset-one for each feature set. For analyses that subsequently used a subset of the CODICES cohort (e.g., lung cancer samples versus healthy controls), the unsupervised batch-corrected, normalized dataset was subset to those samples of interest prior to additional analyses. This meant that a single version of the data, once normalized, was used for all downstream analyses for each feature set; in other words, subsetting after batch correction avoided creating a multiplicity of similar-but-slightly-different datasets for every subset.

### CODICES Cohort: Plasma Proteome Data Generation and Normalization

The Bio-Plex 200 platform (Bio-Rad, Hercules, CA) was used to assess levels of target proteins in human plasma samples. A total of 99.22% (1022/1030) of samples in the CODICES cohort had sufficient plasma to obtain protein concentrations. Briefly, plasma samples were centrifuged, diluted 1:2, and subjected to Milliplex bead-based immunoassays (Millipore Sigma, Burlington, MA), following manufacturer's protocol. The Milliplex HCCBP1MAG-58K (Millipore Sigma, Burlington, MA) panel was used to detect carcinoembryonic antigen (CEA) and osteopontin (OPN). Concentration of each protein analyte was determined using 5 parameter logistic curve fit available in the Bio-Plex Manager 6.2 software (Bio-Rad, Hercules, CA) and protein standards provided with the Milliplex assay.

Samples having protein concentrations with out-of-range values (“OOR<” or “OOR>”) were assigned concentrations either 10% greater than (for “OOR>”) or 10% less than (for “OOR<”) the upper or lower limits, respectively, provided by the protein standards. For CEA, the standard's upper limit was 18,556 pg/mL, and the standard's lower limit was 25.45 pg/mL; for OPN, the standard's upper limit was 400,000 pg/mL and the standard's lower limit was 548.7 pg/mL. A total of 32 plates were used to process the CODICES cohort samples for protein analytes. Plate-specific effects were removed among CODICES cohort samples in an unsupervised manner using multidimensional normalization, as described by Hong et al. (43), with their concomitant R package, MDimNormn (v. 0.8.0), using default settings. After these steps, any samples with missing values were assigned “0” concentrations to avoid sample dropout in downstream machine learning analyses, which could not utilize missing data.

### Sensitivity and Specificity Analyses and ROC Comparisons

Sensitivities at fixed specificity and specificities at fixed sensitivity were analyzed using 1000 bootstrap resamplings, using the approach described by Chabon et al. (“Integrating genomic features for non-invasive early lung cancer detection” Nature. 2020; 580:245-51). When plotting, the median and interquartile range across the bootstrapping resamplings were plotted. It was noted that two other diagnostics classifiers in the field have reported their performances as either ‘specificity at 97% sensitivity’ or ‘sensitivity at 80% specificity’, which is why the model test is also evaluated at those cutoffs. ROC comparisons were performed using Delong's method with the pROC (v. 1.18.0) R package. Since predictions were made on the same samples using models with different feature sets, meaning that the predictions are related to each other, sensitivity and specificity comparisons were calculated on non-bootstrapped data using McNemar's test.

### TCGA Analyses

Bioinformatic alignments of 15,512 strictly host-, base-quality-, and length-filtered (>45 bp) TCGA samples against the metagenomic bins showed that 14,809 samples (95.47%) had ≥1 alignment against 1545 of the bins. Normal adjacent tissues (NATs), which were not part of the metagenome assembly, comprised 73.26% (515/703) of the dropped samples. The remaining samples were then quality controlled for metadata; specifically, (i) 38 samples were removed from Johns Hopkins, solely composed of acute myeloid leukemia, since it would not be possible to distinguish possible sequencing center batch effects from biological effects, and (ii) samples missing sequencing center information were removed (n=22). The remaining 14,749 samples were then subset to those sequenced on Illumina HiSeq machines, which accounted for 98.00% (14454/14479) of the samples, leaving 14454 samples for downstream analyses. Metagenomic bin abundances in TCGA were then analyzed using methods described in detail by Narunsky.

Briefly, batch-correction, when applied, was done using Voom to transform discrete counts to pseudo-normally distributed data, followed by SNM to remove the batch effect(s) in a supervised manner. The only supervised information used was “sample type” (e.g., “blood derived normal”, “primary tumor”, “solid tissue normal”), and batch correction factor(s) comprised sequencing center (“data_submitting_center_label”) and experimental strategy (“experimental_strategy”) when applicable.

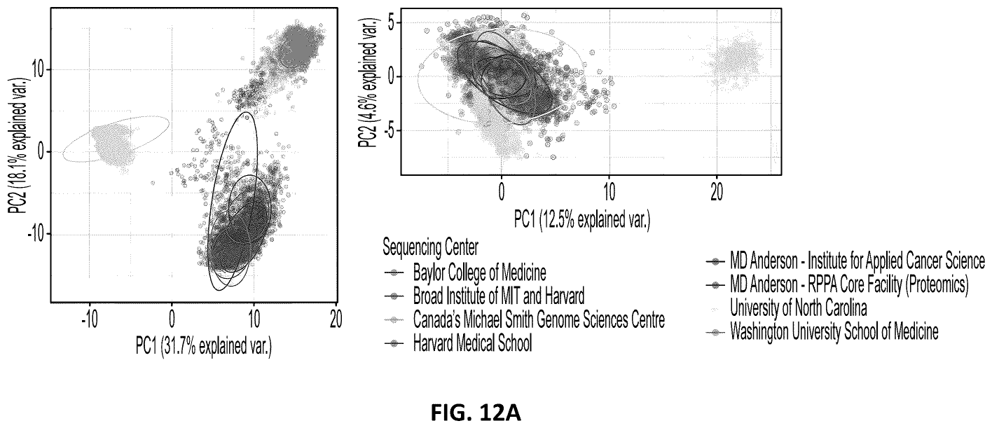

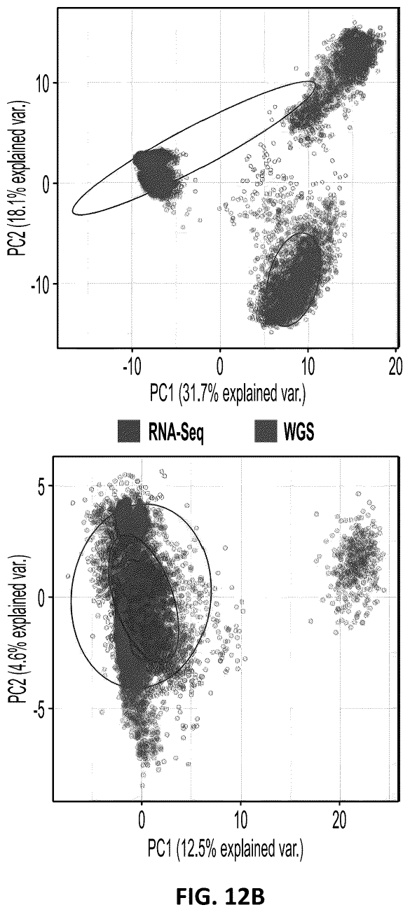

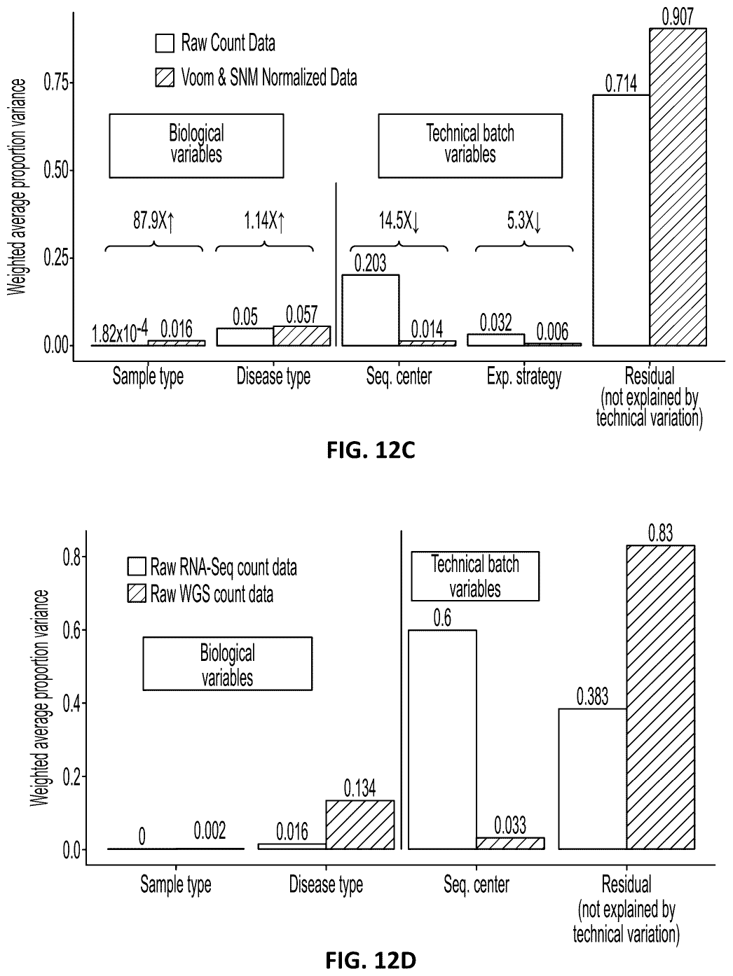

Raw count data were separately tested after either (i) subsetting to individual sequencing centers and experimental strategies or (ii) subsetting only to WGS data since it had lower technical batch effects than disease type (FIG. 12D).

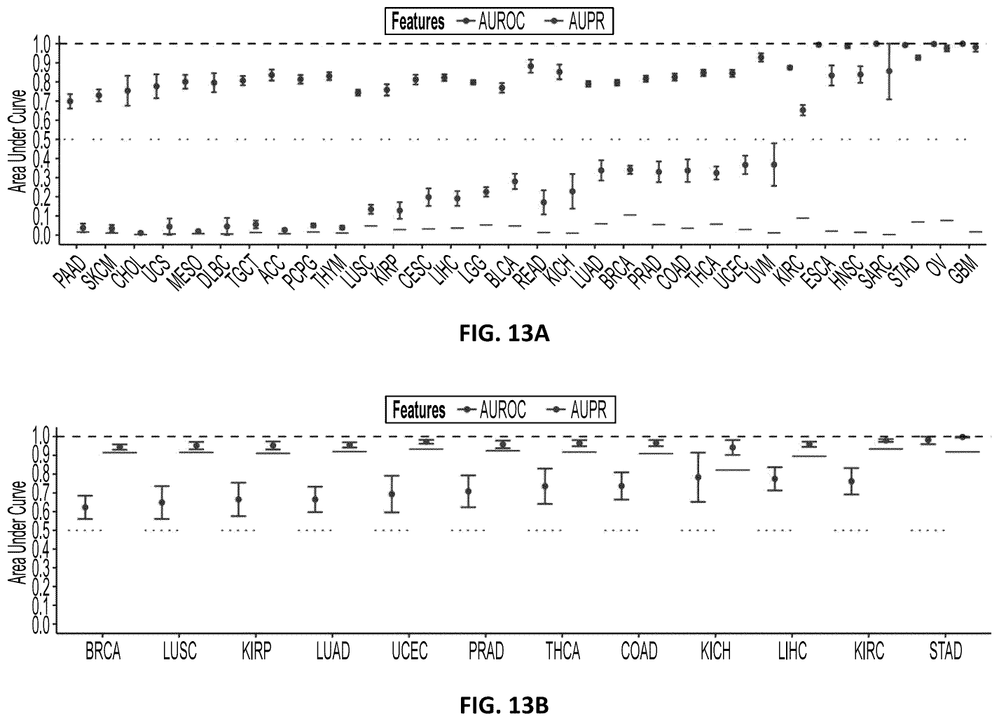

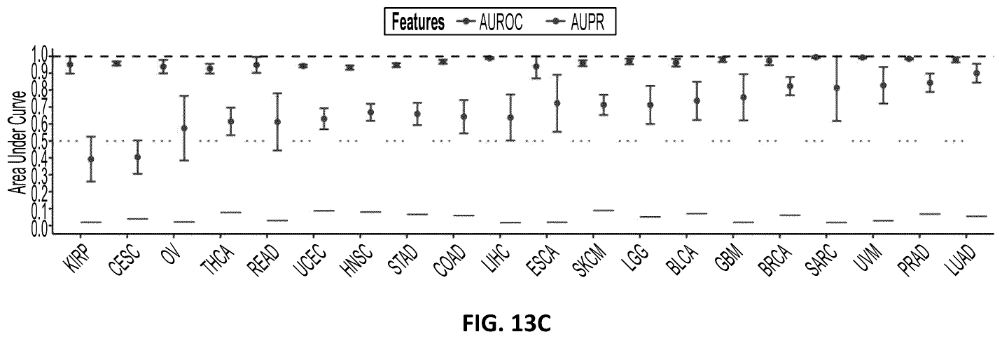

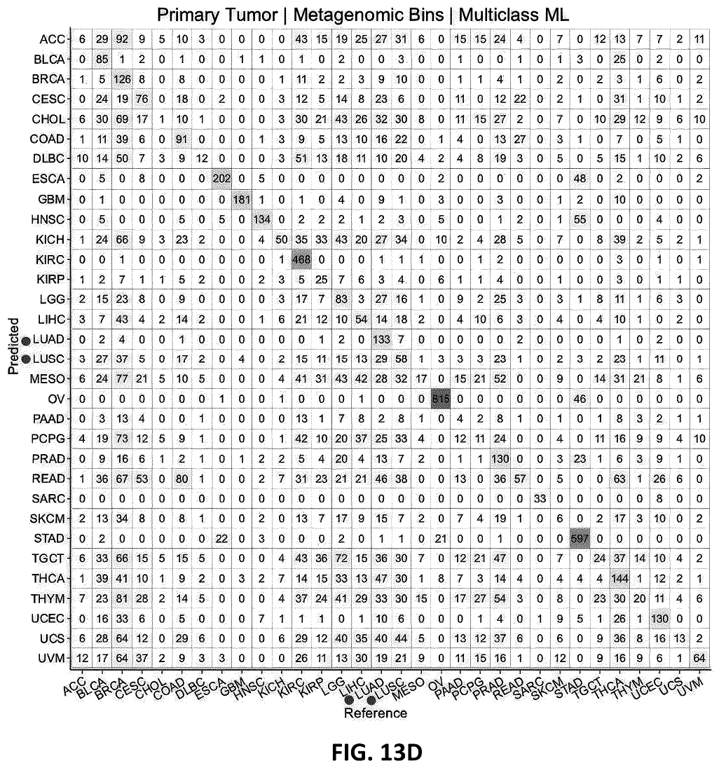

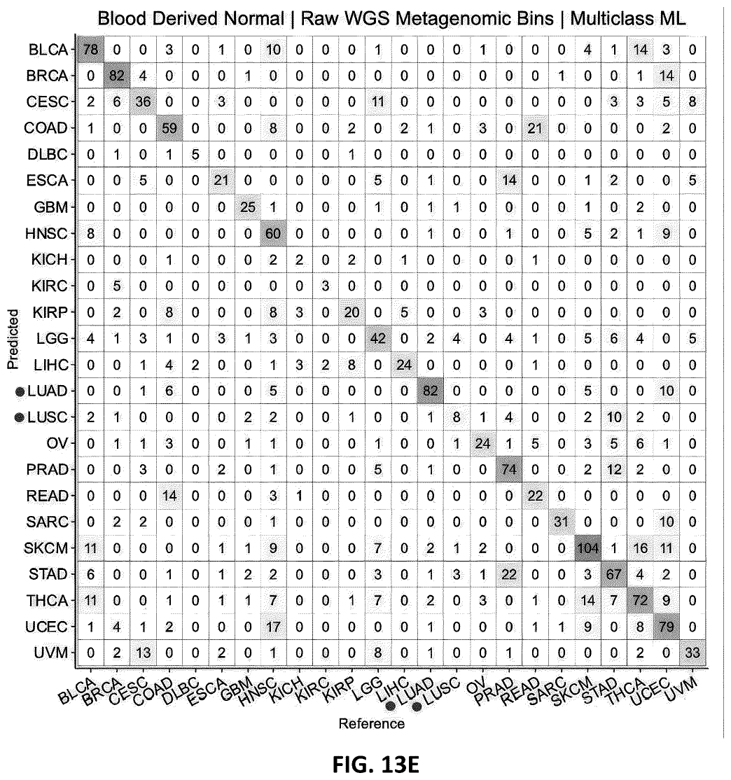

Machine learning was performed with gradient boosting models (GBMs) using 10-fold cross-validation with ten independent, stratified 10% holdouts. ROC and PR curves and areas were calculated for each independent 10% holdout test set, such that ten sets of two-class discriminatory performance—effectively ten sets of 90% training-10% testing—were obtained for each model. The hyperparameters for the GBMs were fixed: {n.trees=150, interaction.depth=3, shrinkage=0.1, n.minobsinnode=1}. These performance estimates on the ten folds were then aggregated for each model to estimate the 95% confidence intervals of performance. In cases of class imbalance, upsampling of the minority class was used; however, ≥20 samples per class were required to run any two-class ML comparisons. Two class comparisons involved (i) one cancer type versus all others for primary tumor and blood analyses and (ii) unpaired primary tumor versus adjacent normal. When using Voom-SNM normalized data, the features were centered and scaled prior to ML; when using raw count data, only zero variance features were removed prior to ML building. No explicit feature selection or data transformations were otherwise performed. For multiclass ML that used either batch-corrected data (FIGS. 9I-1 and 9I-2; and FIG. 13D) or raw WGS data (FIG. 13E), Extreme Gradient Boosting (XGBoost) machines were employed with 10-fold cross-validation, removal of zero variance features, and the following fixed hyperparameters: {nrounds=10, max_depth=4, eta=0.1, gamma=0, colsample_bytree=0.7, min_child_weight=1, subsample=0.8}.

Negative control machine learning analyses were run the same as above but either (i) scrambled metadata of prediction labels or (ii) shuffled the sample IDs in the count data dynamically just prior to ML model building. The differences between global scrambling and shuffling (i.e., once before all ML models are built and tested) versus dynamic scrambling and shuffling (i.e., just prior to ML model building but after data subsetting and labeling) were previously tested, and found that dynamic scrambling and shuffling yielded more consistent results (less variance) and showed greater agreement with known null values (i.e., 50% AUROC and positive class prevalence for AUPR). Hence, dynamic scrambling and shuffling as negative controls was used when comparing performance to actual samples. Since the same random number seeds were used for both ML on actual analyses and negative control analyses, they were evaluated on the same cross-validation folds. Downstream statistical analyses compared the performances of actual analyses versus negative control analyses, finding statistically better performances of the former.

Briefly, alpha and beta diversity analyses were run using Qiime 2 (v. 2021.11) and respective plugins on sample subsets comprising individual sequencing centers, WGS, and sequencing platform (Illumina HiSeq). Alpha diversities were calculated using Qiime 2's non-phylogenetic core-metrics function, rarefying to 15,000 reads/sample (approximately 1st quartile of sample read distribution among primary tumors and blood samples). Beta diversity analyses were run using DEICODE's RPCA (robust Aitchison distance), which did not need rarefied data by design, followed by Qiime 2's implementation of adonis to calculate concomitant PERMANOVA statistics.

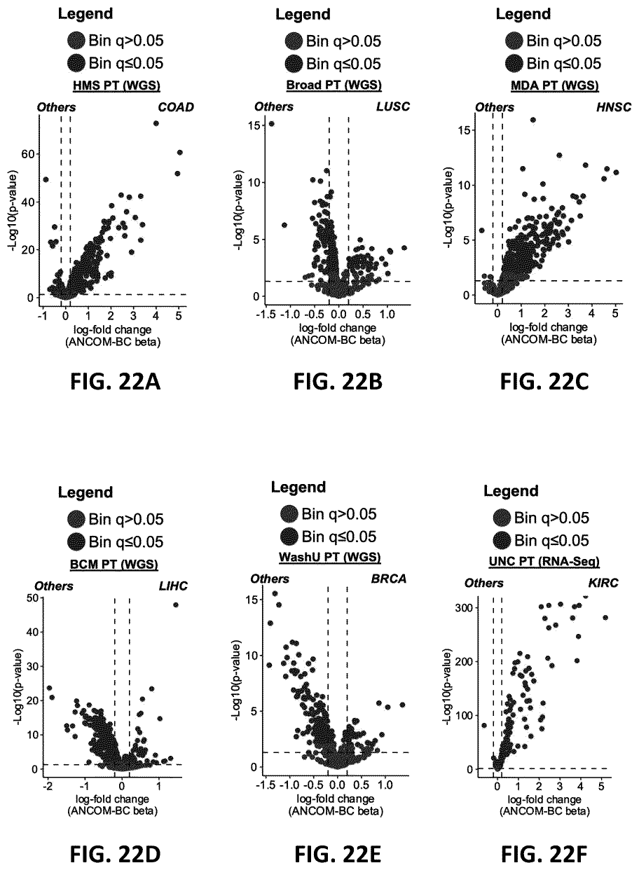

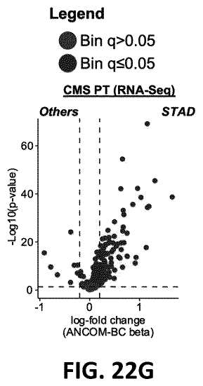

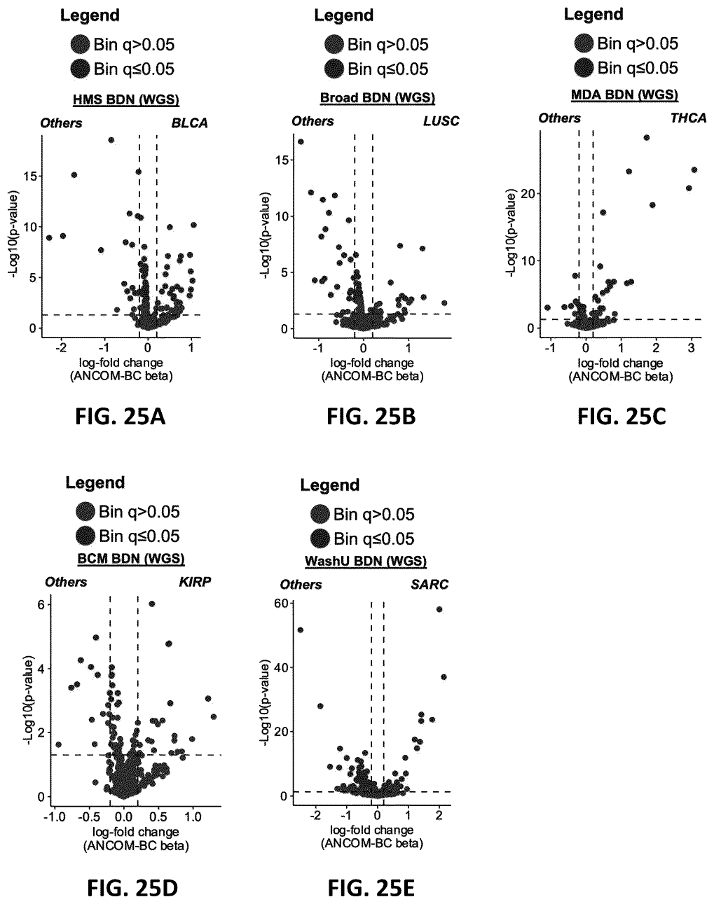

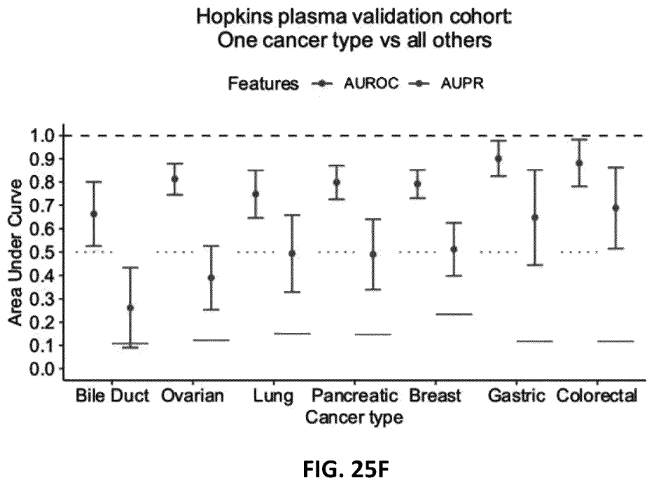

Briefly, for differential abundance testing, ANCOM-BC was iteratively applied within WGS sequencing center subsets to evaluate one-versus-all-others comparisons among cancer types using metagenomic bin abundances in primary tumors (FIGS. 22A-G) or blood (FIGS. 25A-25E). The following parameters were used: {p_adj_method=“BH”, zero_cut=0.999, lib_cut=1000, tol=1e-5, max_iter=100, conserve=FALSE, alpha=0.05, global=FALSE}. A minimum number of 10 samples in each class was enforced before computing differentially abundant taxa; otherwise, the comparison was skipped. Statistical discrimination was done per cancer type versus all others within each subset The calculated beta values, p-values, and BH adjusted q- were then used values to make volcano plots.

### Hopkins Cohort Analyses

Bioinformatic alignments of 537 strictly host-, base-quality-, and length-filtered (>45 bp) samples from Cristiano et al. (“Genome-wide cell-free DNA fragmentation in patients with Cancer.” Nature. 2019; 570:385-9; hereinafter “Hopkins” or “Cristiano”) against the metagenomic bins gave ≥1 alignment for all 537 samples against 770 of the bins. As done previously only treatment-naive samples were used for downstream analyses, and if a patient had ≥1 sample, then the earliest time point sample was selected, collectively leaving 491 samples (91.43% of total) for downstream analyses. Metagenomic bin abundances in the Hopkins cohort were then analyzed using methods described above and in detail by Narunsky.

Briefly, for machine learning on individual cancer versus healthy or one-cancer-type-versus-all-other-types comparisons, GBMs on raw metagenomic bin abundances were applied using 10-fold cross-validation with the following fixed hyperparameter set (same as TCGA): {n.trees=150, interaction.depth=3, shrinkage=0.1, n.minobsinnode=1}. Zero variance features were removed prior to ML and upsampling occurred in cases of class imbalance. Performances on individual, independent holdout folds were used to estimate 95% confidence intervals of AUROC and AUPR.

Briefly, for machine learning comparisons between grouped cancer samples and healthy controls (FIGS. 9M-1 and 9M-2), the same machine learning architecture was employed, but 10 repeats were used of 10-fold cross validation to identically match the approach used by Cristiano et al. In that case, confidence intervals were estimated using the repeats rather than the individual folds. To plot, a scikit-leam approach (https://scikit-leam.org/stable/auto_examples/model_selection/plot_roc_crossval.html) for R was adapted to estimate the average AUROC and AUPR curves among their 10 repeated iterations. This can be a challenging task because the specificity breaks of the ten model iterations are not always equivalent to each other, requiring interpolation. Specifically, to obtain the average performance lines, linear interpolation using the approx( ) base R function of each ROC and PR curve across 1000 equally spaced points between 0 and 1 was performed, also ensuring that each average curve begins and ends at the corners of the plots. The 1000 interpolated y-values between x=0 and x=1 was then used to calculate the average ROC and PR curve and its concomitant 95% confidence interval at each point. Overlaying these average performance lines with 95% confidence interval ribbons showed good concordance

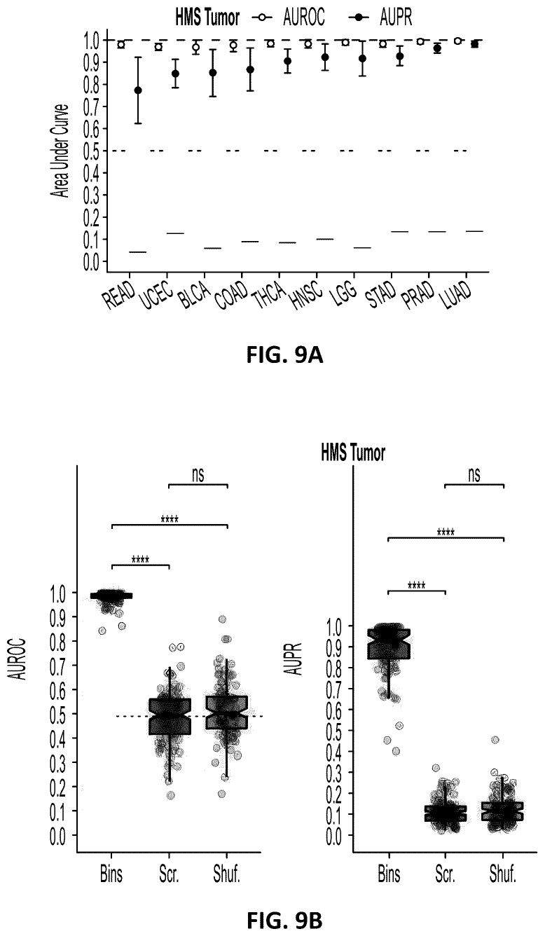

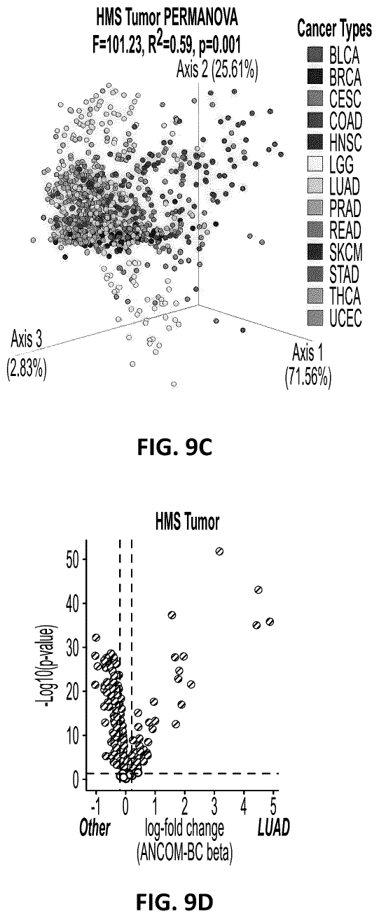

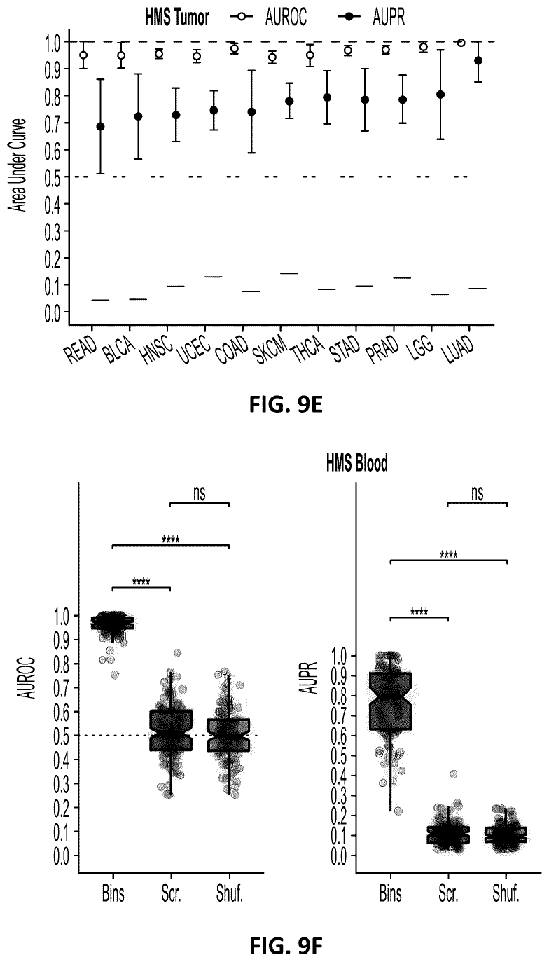

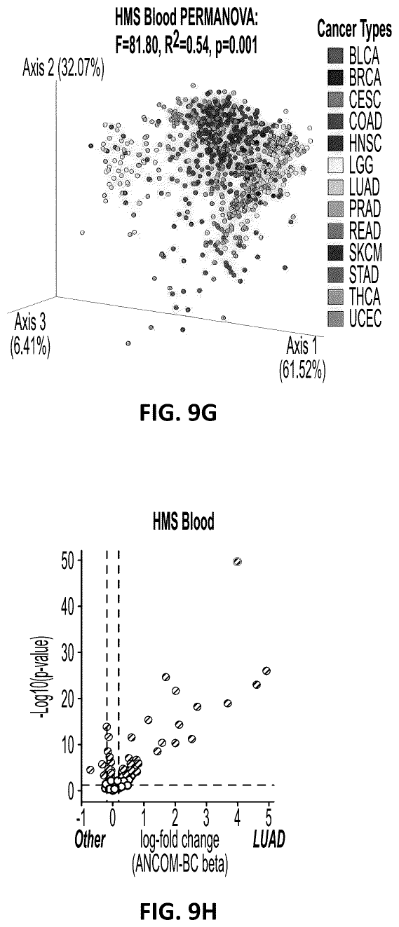

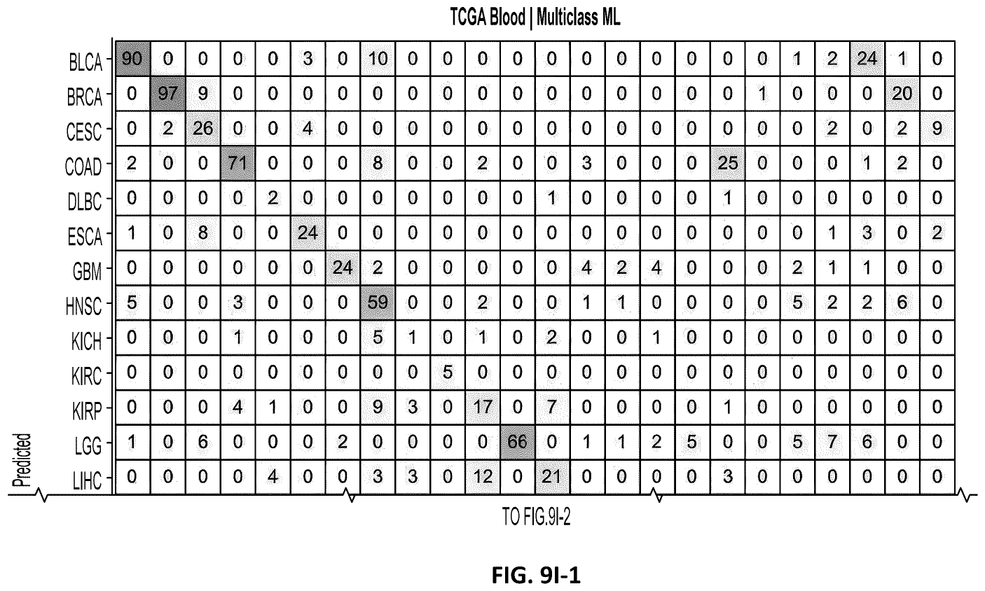

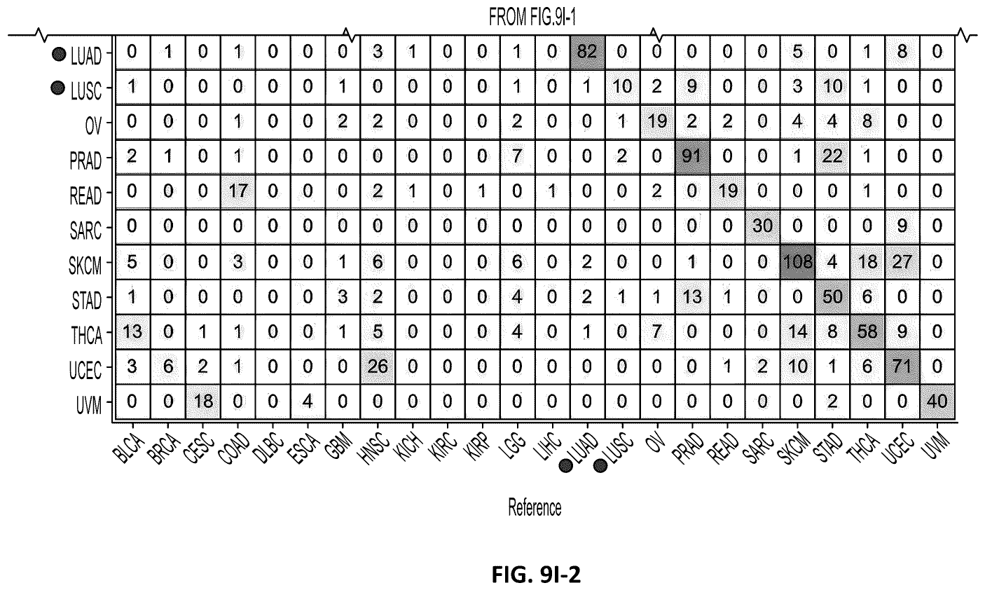

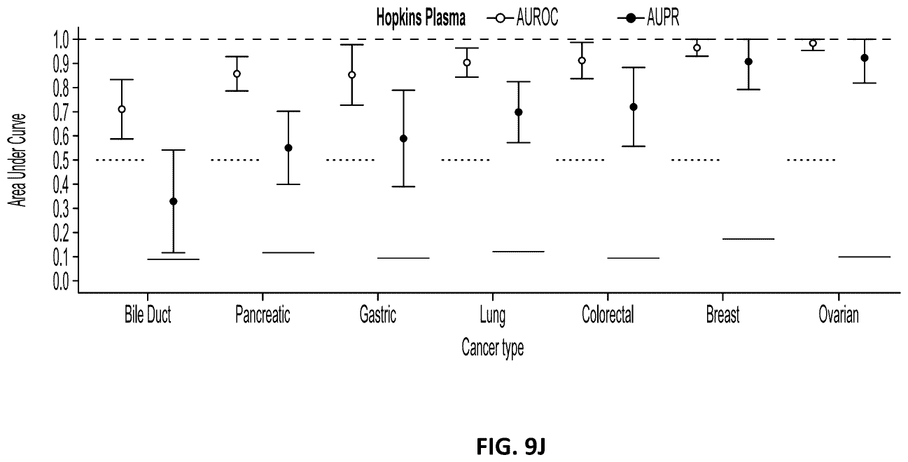

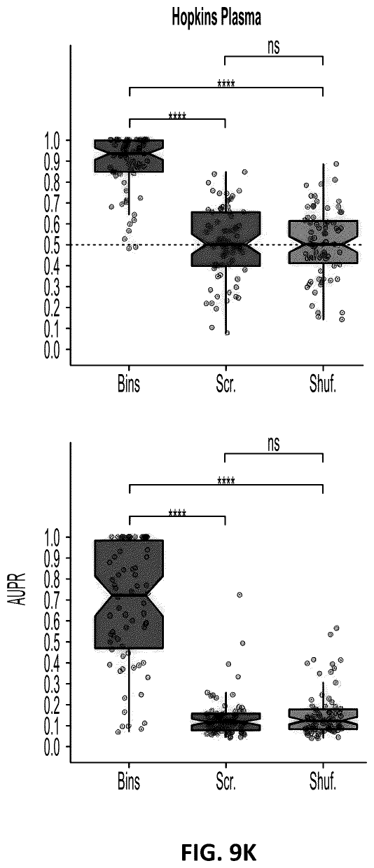

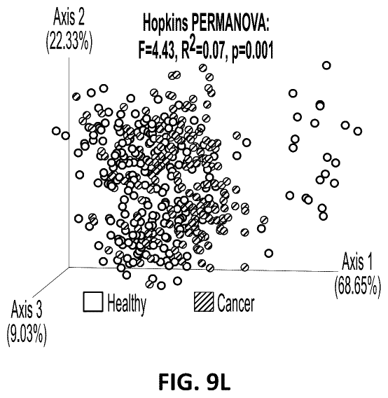

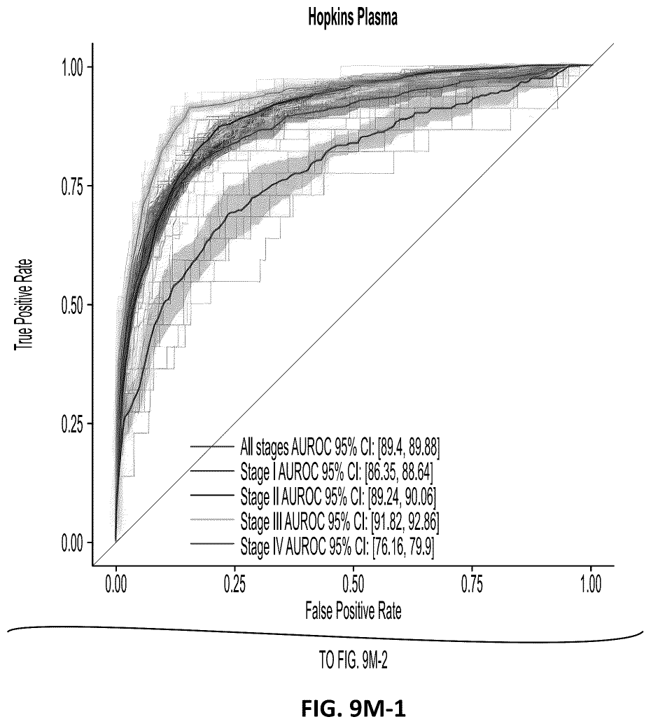

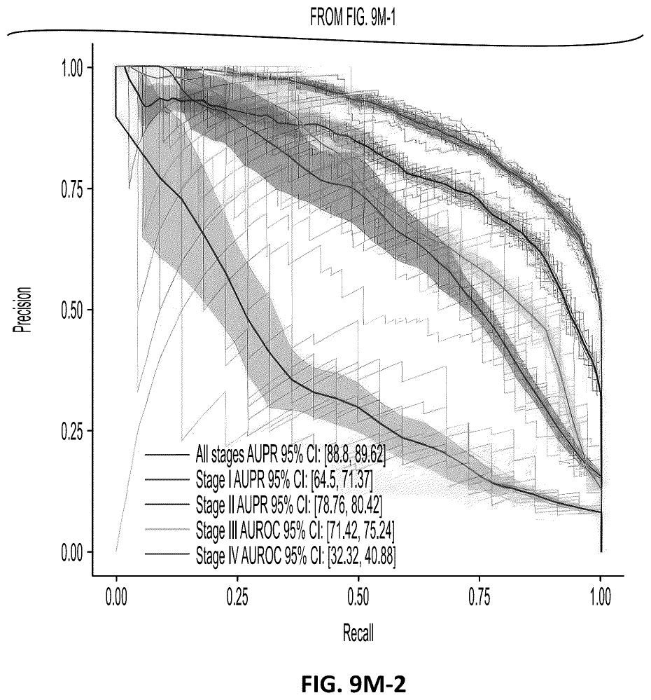

For negative control ML analyses, samples either had their metadata dynamically scrambled or their count data dynamically shuffled, as done in TCGA, using the same random number seed to ensure matched cross-validation fold comparisons with non-scrambled/shuffled analyses. Statistical comparisons between actual and control analysis performances were then performed, showing significantly better performance for actual samples (FIG. 9K).

For beta diversity, DEICODE's RPCA (robust Aitchison distances) was applied (same as TCGA and CODICES cohorts), using Qiime 2's DEICODE plugin, followed by calculating PERMANOVA statistics with Qiime 2's implementation of adonis.

### Hong Kong Liver Cancer (HCC) Cohort: Data Accession and Processing

Sequence data of plasma from liver cancer patients and healthy individuals was downloaded from the European Genome-Phenome Archive (EGA) accession EGAS00001001024.42 Reads were quality filtered by fastp, host depleted against GRCh38, and then aligned to the metagenomic bins using Salmon. From the metagenomic bins, alpha diversity, beta diversity by Jaccard and RPCA, 51 and differential bin abundance by ANCOM-BC.44 Statistical differences in alpha diversity were calculated by Wilcoxon test and beta diversity metrics by PERMANOVA. Nucleotide frequency (NTF) data were generated by Budhraja et al. (PMID: 36630480) and their combination with bin abundances were used for downstream machine learning analyses.

### Fragment-End Motif Nine Nucleotide Frequencies (NTFs) Analysis

Nucleotide frequency information around fragment ends were calculated using quality filtered sequencing reads before human read removal. The nucleotide frequency of nine relative positions around the read fragment ends, which were previously identified to be most informative for cancer determination (PMID: 36630480) were calculated. This resulted in a table with the frequency of each base at each of the nine relative positions for each sample. This information was used alongside microbial, proteomic, and/or clinical data for downstream machine learning analyses.

### Lung-Gut Cohort: Data Accession and Processing

16S rRNA sequence data of stool from LUAD-bearing patients and healthy individuals was downloaded from the European Nucleotide Archive (ENA) accessions PRJEB44169 (LUAD samples) and PRJEB33905 (healthy samples, PMID: 34586729). Reads were quality filtering by fastp, host depleted against GRCh38 (for consistency, it was not expected that human reads would be in 16S data), and then aligned to the metagenomic bins using Salmon. From the metagenomic bins abundances, we calculated alpha diversity, beta diversity by Jaccard and RPCA (PMID: 30801021) and differential bin abundance by ANCOM-BC (PMID: 32665548). Statistical differences in alpha diversity were calculated by Wilcoxon test and beta diversity metrics by PERMANOVA.

### Blinded Validation Cohort Analysis

The blinded validation cohort comprised 108 samples that were sent with plasma aliquots ≤1 mL and paired clinical metadata without labels or diagnoses. Plasma was processed using identical methods described above for the CODICES cohort to obtain shotgun metagenomic data on an independent sequencing run. Small aliquots of plasma were also processed for proteomic information (CEA, OPN). Clinical metadata was normalized identically to that in the CODICES cohort (described above), including calculation of the Mayo clinical risk score, pCA. After reviewing the metadata, two samples were discarded on the basis of metadata quality, specifically for having a missing age (“0”) or an unverifiable age (year ending in “00”, implying either 22 years old or 122 years old, either the youngest or oldest outlier in the cohort), which would have affected the pCA calculation. This left 106 blinded samples on which to evaluate the clinico-proteo-metagenomic test.

Prior to performing predictions on the blinded cohort, possible sequencing run variation and protein-plate variation using an unsupervised normalization approach was taken into account. Specifically, for the metagenomics data, the following steps were taken: (i) raw metagenomic bin abundances from the 454 samples in the CODICES cohort with available clinico-proteo-metagenomic information were row-binded with raw data from the blinded validation cohort; (ii) unsupervised ComBat-Seq was run on the merged table with the “batch” vector comprising sequencing runs for the 454 CODICES samples and the independent validation run, using no other information and default settings; (iii) the metagenomic bin normalized 454 CODICES samples with available clinico-proteo-metagenomic information were then set aside for model training; (iv) the normalized metagenomic bin data for blinded validation cohort samples were set aside for model predictions; and (v) the set-aside normalized metagenomic bin data for the blinded validation cohort samples were independently center-log-ratio transformed.

For the proteomic data, following steps were taken: (i) blinded validation cohort samples having protein concentrations with out-of-range values (“OOR<” or “OOR>”) were assigned concentrations either 10% greater than (for “OOR>”) or 10% less than (for “OOR<”) the upper or lower limits, respectively, provided by the protein standards (as done for the CODICES cohort); (ii) protein concentrations from the blinded validation cohort were then row-binded to raw protein concentrations from the 454 CODICES samples; (iii) plate-specific effects were then removed from the combined protein data in an unsupervised manner using multidimensional normalization; (iv) any samples in the CODICES cohort with missing values were assigned “0” concentrations to avoid sample dropout (Note: all blinded validation cohort samples had available protein concentrations); (v) normalized protein concentrations for the 454 CODICES samples were set aside for model training; (vi) normalized protein concentrations for the blinded validation cohort samples were set aside for model predictions.

For the blinded validation cohort's clinical metadata, the data was normalized identically to the CODICES cohort. For any clinical variable instances found in the CODICES cohort that were not found in the blinded validation cohort (e.g., tumor_solidity=“part solid and ground glass” was an instance only in the CODICES cohort), then an empty, zero-valued dummy variable column was added to the blinded validation cohort clinical metadata. This was necessary specifically since (a) the stacked ML approach used dummy variables for categorical and boolean features, and (b) the stacked ML tuned model requires the same features to be present to make predictions as originally used for training.

With the CODICES cohort's metagenomic and proteomic data normalized (n=454 samples), the final model was tuned using the 820 stepwise hyperparameter grid search described above using these data in tandem with clinical metadata. Specifically, the following features were included: metagenomic bins, CEA, OPN, pCA probability, Brock probability, smoker status, emphysema status, tumor size (cm), spiculation, tumor solidity, upper lung nodule. As also done before, the metagenomic data were center-log-ratio transformed immediately prior to the stacked ML. Additionally, equivalent stacked ML models on the same samples using only PET-CT SUVs, pCA probabilities, or Brock probabilities was built.

The final trained CODICES cohort stacked ML model, was then applied on the normalized, blinded validation cohort's clinico-proteo-metagenomic data to obtain probability predictions on each sample. this process for the CODICES cohort stacked ML models built using only PET-CT SUVs, pCA probabilities, or Brock probabilities was then repeated. The probability predictions were then sent to the provider of the blinded cohort, and the ROC curve and respective AUROC were returned.

CODICES, 16S rRNA, TCGA, Hopkins, and Validation Cohorts: Statistical Analyses

Downstream analyses and plots were generated with either R version 4.03 or 4.1.1. Common R packages used include phyloseq (v. 1.38.0), vegan (v.2.5-7), doMC (1.3.7), dplyr (v. 1.0.7), reshape2 (v. 1.4.4), ggpubr (0.4.0), ggsci (v. 2.9), rstatix (v. 0.7.0), tibble (v. 3.1.6), caret (v. 6.0-90), caretEnsemble (v. 2.0.1), gbm (v. 2.1.8), xgboost (v. 1.5.0.1), randomForest (v. 4.6-14), glmnet (v. 4.1-3), MLmetrics (v. 1.1.1), PRROC (v. 1.3.1), pROC (v. 1.18.0), e1071 (v. 1.7-9), gmodels (v. 2.18.1), ANCOM-BC (v. 1.4.0), decontam (v. 1.14.0), limma (v. 3.50.0), edgeR (v. 3.36.0), snm (v. 1.42.0), sva (3.35.2), biomformat (v. 1.22.0), and Rhdf5lib (v. 1.16.0). The rstatix package corrected for multiple hypothesis testing where applicable. Sample sizes were not estimated in advance and power calculations were not performed. The gbm, randomForest, and glmnet packages were used for two-class ML; the xgboost package was used for multi-class gradient boosting ML. AUROC and AUPR were calculated using the PRROC package for all but the validation analyses, which used pROC. It was noted that the R programming language has two numerical limits when it comes to calculating small numbers, including p-values: (1) double eps, or smallest positive floating-point number x such that 1+x !=1, which is 2.220446×10⁻¹⁶; (ii) double xₘᵢₙ, or the smallest non-zero normalized floating-point number, which is 2.225074×10⁻³⁰⁸ (although this limit may be even lower depending on the computing environment). Some R packages, notably ggpubr, do not report p-values less than double eps, so they are denoted in our data as p<2.2×10⁻¹⁶; conversely, other R packages, notably rstatix (listed below), report p-values as low as double xₘᵢₙ, and p-values that were less than double xₘᵢₙ in our data are reported as p<2.2×10⁻³⁰⁸. They are not a range of p-values.

### Plasma Microbiome Discovery Cohort

To more comprehensively capture the distribution of the plasma microbiome and evaluate its diagnostic usefulness, age- and sex-matched CODICES cohort (Comprehensive Oncobiome analysis for Diagnostic Identification of Cancer in Early Stages), comprising 1030 treatment-naive plasma samples across 11 pathological subtypes of lung cancer (38.93%), 11 diverse etiologies of lung disease (21.55%), and healthy controls (39.51%) were constructed. Among lung cancers, nearly two thirds of them were clinical stage I (n=131, 32.59%) and stage II (n=110, 27.36%), with stage III (n=98, 24.38%) and stage IV (n=57, 14.18%) comprising smaller subsets. Smoking status (i.e., current, former, never) was known for 708 (68.73%) subjects, enabling sub-analyses within smokers, who carry the most common risk factor for lung cancer. Ethnically diverse populations, with 15.63% (n=161) of the samples stemming from subjects with self-defined Black or African American, American Indian, Asian, or Pacific Islander ethnicities were also captured. Additionally, 428 (41.55%) of the subjects had radiological-informed lesion sizes, both for lung cancer and lung disease, with a median diameter of 2.5 centimeters (avg±SD=2.96±1.91 cm). To complement the CODICES discovery cohort, a blinded validation cohort comprising 106 patients with either clinical stage I cancer or lung disease was separately acquired.

400 μL of plasma from each subject in the CODICES cohort, followed by extracting cell-free DNA and performing shallow shotgun metagenomic sequencing (˜20 million reads/sample) was then isolated. Approximately a third of the samples (n=335) were additionally processed for 16S rRNA amplicon sequencing using the shorter V6 region, due to the highly fragmented nature of cell-free DNA, on a single sequencing run as validation of the approach. To control for contamination, every sequencing run for both shotgun metagenomic and amplicon data contained positive (mock communities) and negative (experimental blank) controls, in conformity with other low-biomass microbial sequencing protocols. Sample types were also randomized on every sequencing run to prevent confounding by batch. Furthermore, since multiple host-centric diagnostic approaches have described utility of protein-based markers in lung cancer, the plasma-derived concentrations, when volumes permitted, of carcinoembryonic antigen (CEA) and osteopontin (OPN), covering 99.22% (n=1022) of the CODICES cohort were also measured.

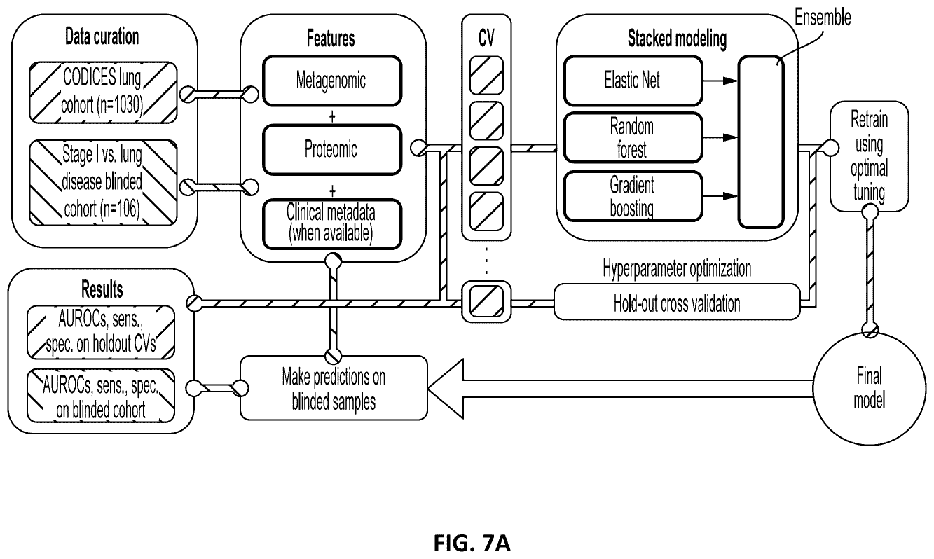

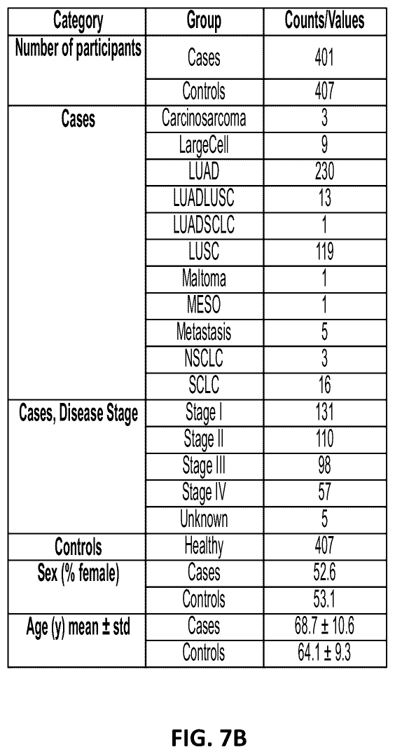

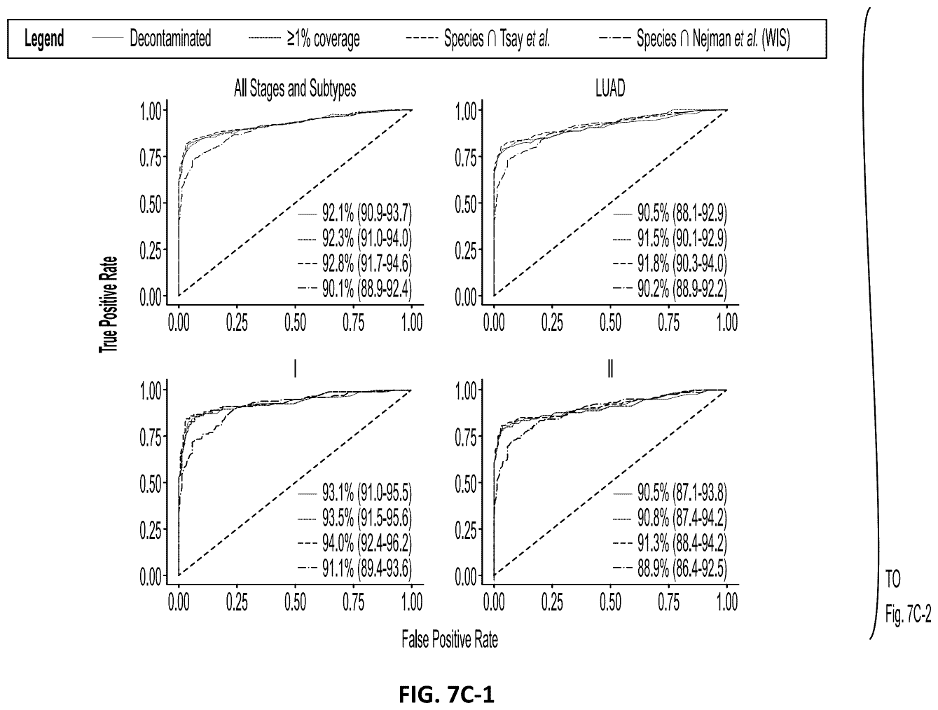

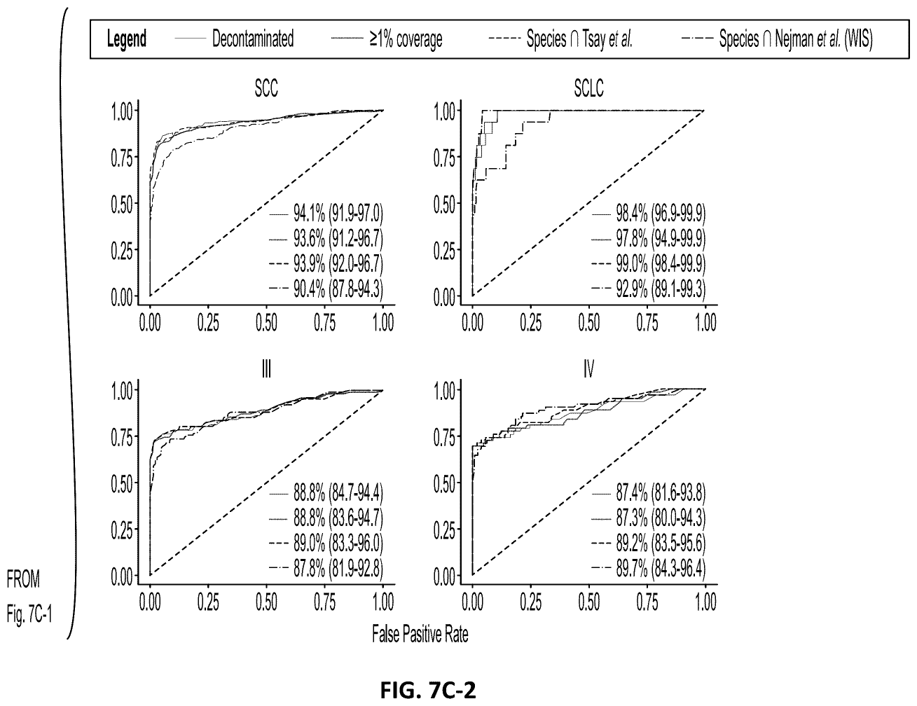

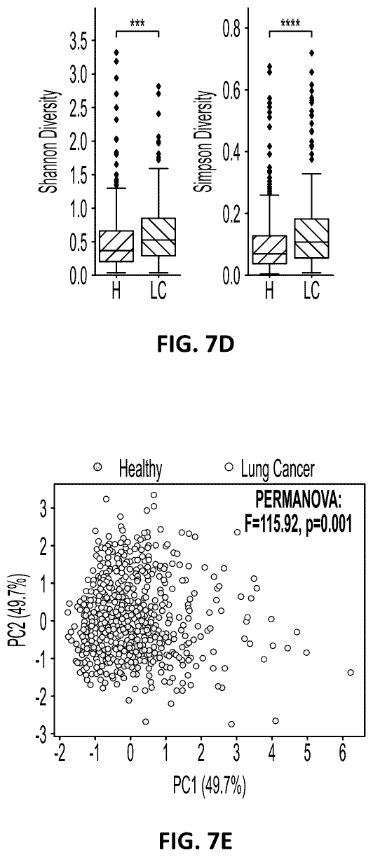

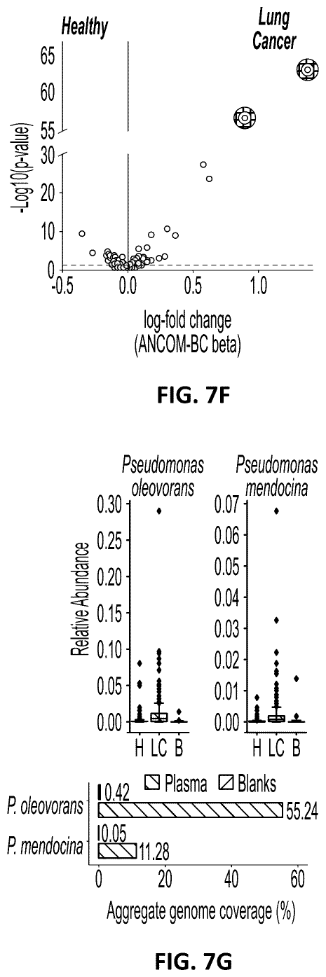

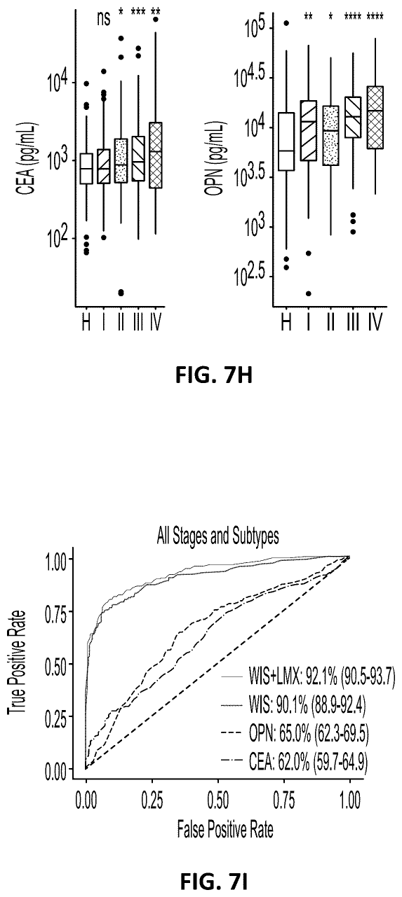

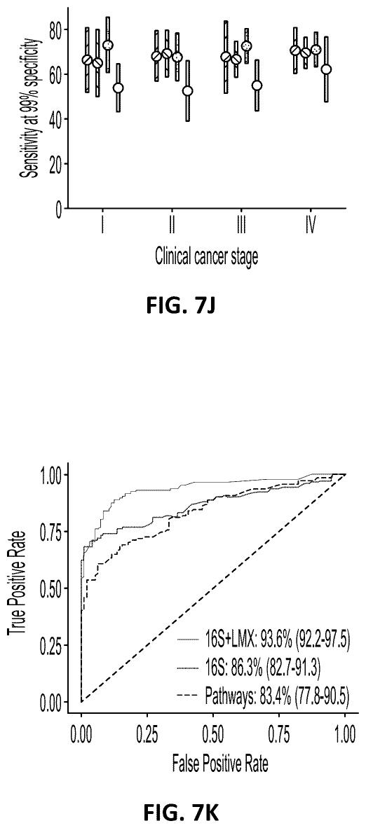

The availability of matched metagenomic, proteomic, and clinical metadata prompted development of a multi-species (host and microbial) stacked machine learning (ML) strategy (FIG. 7A). Specifically, after filtering out low varying features and center-log-ratio transforming microbial counts, concatenated data were fed into an ensemble classifier comprising elastic net, random forest, and gradient boosting algorithms acting in parallel using matched cross-validation (CV) folds. Scores from the three algorithms were then weighted using a logistic regression ensemble model. A ten-fold CV scheme was employed to optimize model hyperparameters within an 820 step-wise grid search. Predictions on the independent holdout cross-validation folds were saved to estimate modeling performance on the discovery cohort, and the final, clinico-proteo-metagenomic model tuning occurred on 454 CODICES samples that had all three sets of available information, prior to application on the 106-patient blinded validation cohort (FIG. 7A).

### Evaluating Known Metagenomic Features for Lung Cancer Diagnosis in the Clinical Low-Risk Setting

With the sequencing data and ML architecture in place, the diagnostic capacity of known metagenomic features in the low-risk clinical setting of 407 healthy versus 401 treatment-naive, age and sex-matched, cancer-bearing individuals across all stages (FIG. 7B) was evaluated. Specifically, all 9.57×108 cell-free DNA reads that did not map to the human reference genome (1.82% of total) were aligned against the against the RefSeq release 206 database (“rep206”) containing bacterial, fungal, and viral genomes, with a total of 9.60×107 reads aligning to microbes (0.18% of total). putative contaminants were then inferred using 98 total extraction blanks that were processed in parallel and included on every sequencing run. Prevalence-based filtering using these blanks identified and removed 5630 species (46.59% of taxa, n=6455 taxa remaining), which were predominantly low-abundance taxa, accounting for just 0.88% of total microbial reads (FIG. 11A). Since rep206 genomes are well defined, the aggregated genomic coverage was calculated using every sample in the CODICES cohort, across every microbe in the database with Zebra (FIG. 11B). Then a new subset of microbial taxa having ≥1% aggregate genomic coverage (n=2172, 17.97% of taxa) to compare downstream ML performances was created. Repeating these steps using microbes identified in experimental blanks revealed that many well-covered microbes in plasma had low coverage in blanks (FIG. 11C).

Next the rep206-identified microbes were intersected with those previously found in lung cancer-associated bronchoalveolar lavage (BAL) samples (n=6530 taxa), as well as bacterial and fungal species identified in two highly decontaminated, pan-cancer, tissue-centric studies from the Weizmann Institute of Science (WIS) (n=300 taxa). Notably, when calculating Fisher's exact test for enrichment in the ≥1% aggregate genome coverage set, both the BAL sample features (p=3.37×10-147, X2=667.57) and WIS features (p=2.44×10-59, X2=263.89; FIG. 11B) demonstrated strong overlap despite their BAL and tissue origin, respectively, suggesting that plasma-derived microbial features may compositionally reflect their intratissue and even peri-tissue counterparts.

The diagnostic performance of these four microbial feature sets using our stacked ML strategy (FIG. 7A and FIGS. 7C-1 and 7C-2) was then compared. Remarkably, although these feature sets varied 21.8-fold in size between the largest and smallest (6530 vs. 300 taxa), all of them provided strong and similar AUROCs (range: 90.1-92.8%), with overlapping 95% confidence intervals (FIG. 7C-1, upper left). Subsetting predictions to varying histological subtypes and stages further showed consistently strong diagnostic performances (min AUROC: 87.3%, max: 99.0%), with the strongest performance for small cell lung cancers (SCLC, AUROCavg=97.03%; FIG. 7C-2, upper right). Unexpectedly, when examining predictions across all cancer subtypes in individual stages, the best diagnostic performance was observed when distinguishing clinical stage I lung cancers against non-cancer controls (Stage I AUROCavg=92.93%; FIG. 7C-1, bottom left), followed by a slight but stepwise decline in performance at higher stages (Stage IV AUROCavg=88.4%; FIG. 7C-2, bottom right). It is possible that this trend either reflects fewer samples available at higher stages in the CODICES cohort (FIG. 7B) or a biological phenomenon, wherein late-stage cancers may sufficiently disrupt the vascular barrier to allow non-cancer-associated microbial DNA into circulation. Nonetheless, even in treatment-naive contexts, these analyses validate and extend our original observations that cell-free microbial DNA (cf-mbDNA) provides a novel class of cancer diagnostics to 8.6-fold more samples than previously examined.

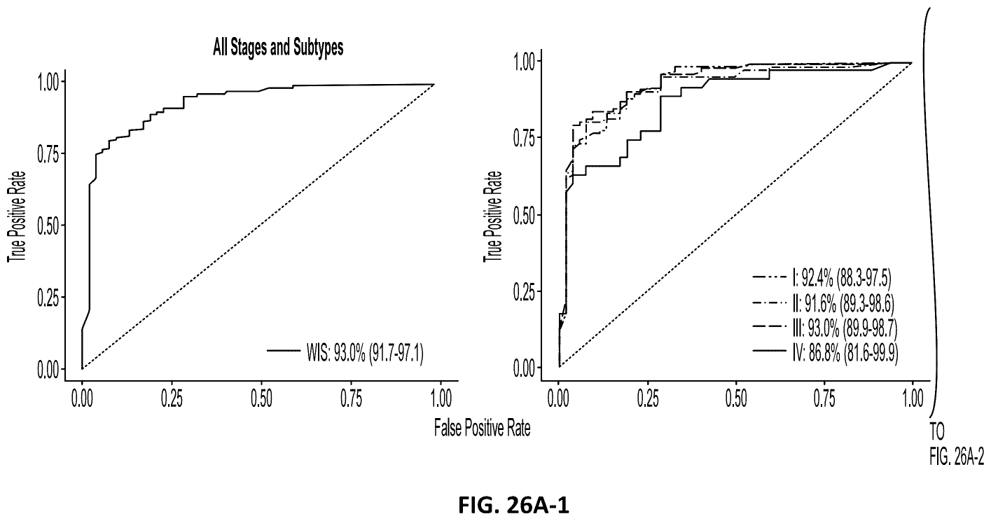

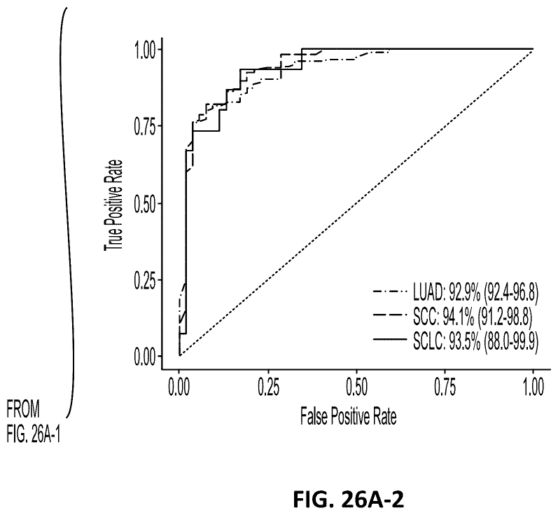

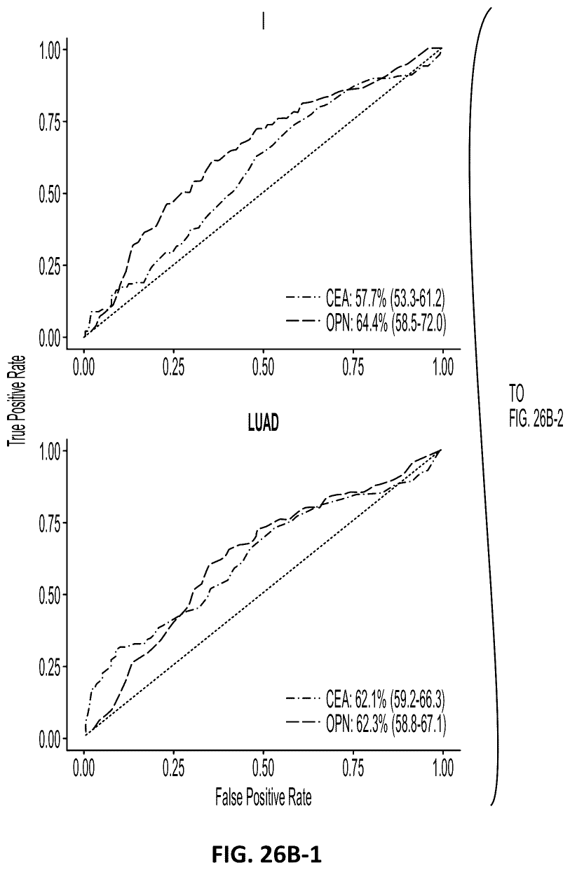

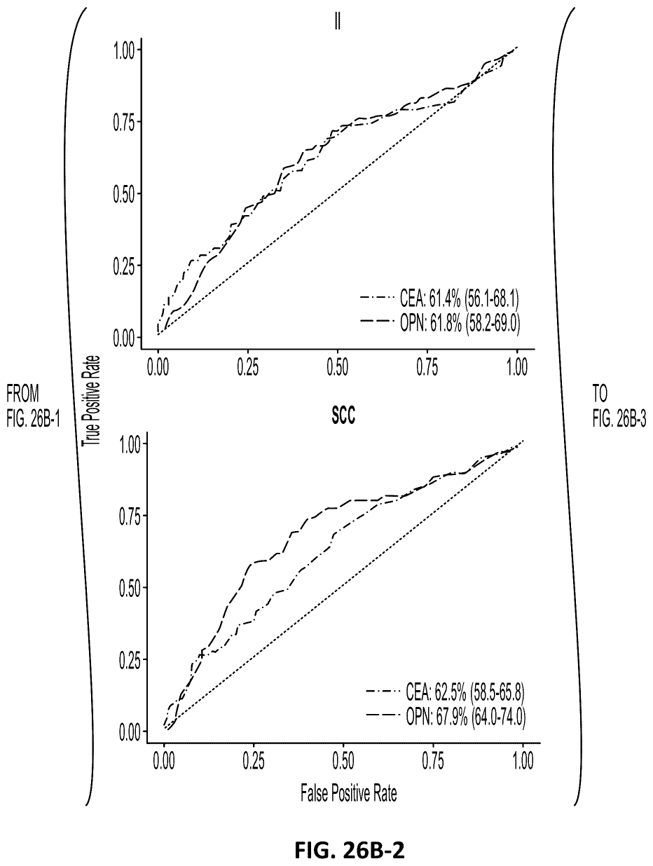

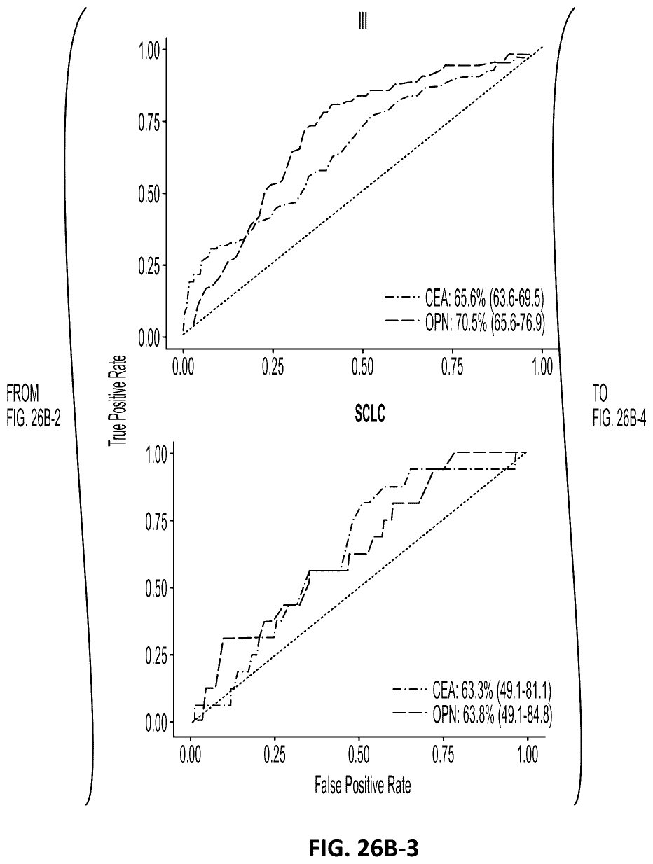

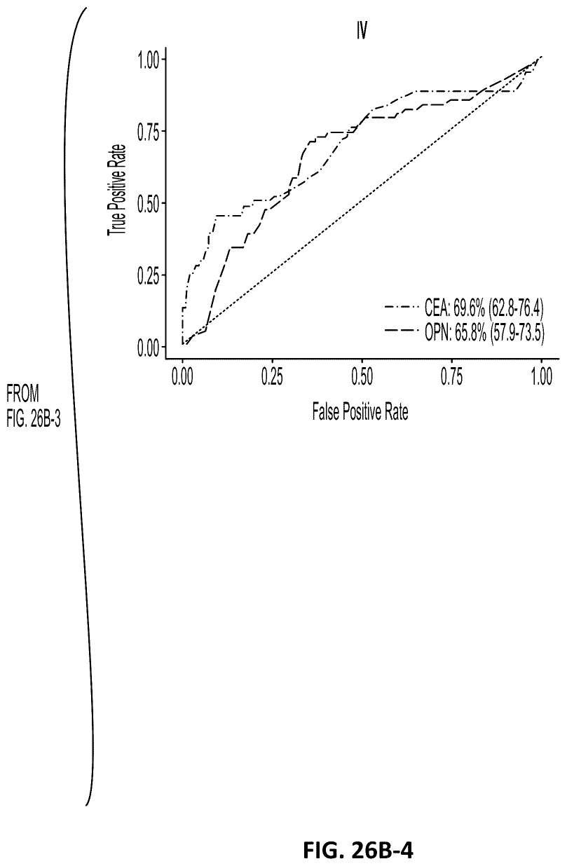

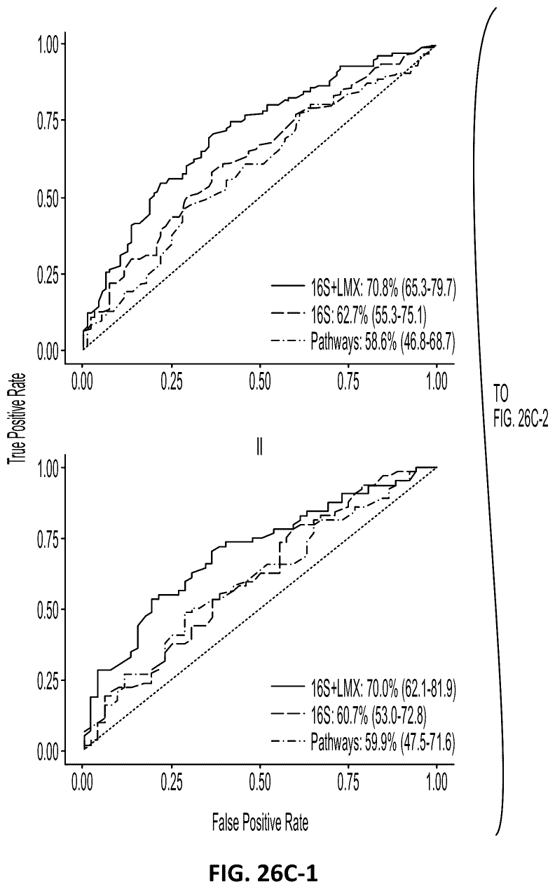

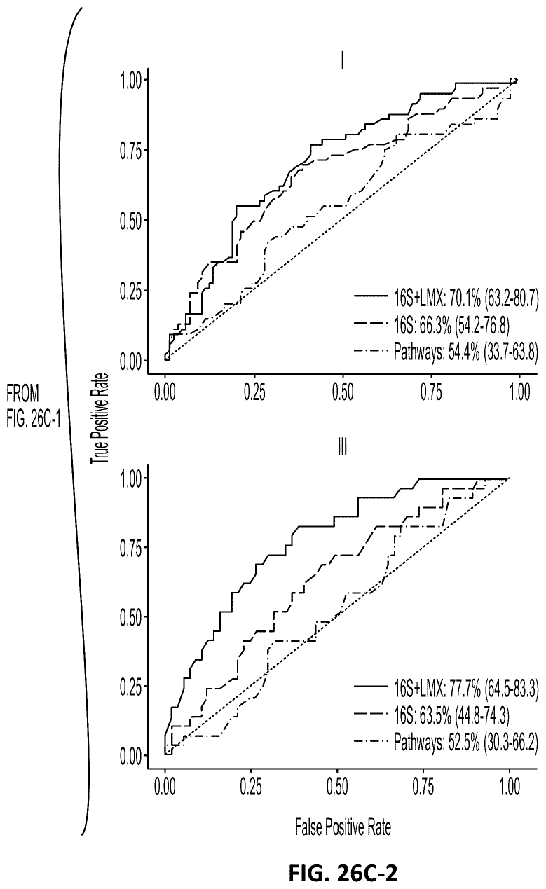

Since the WIS-overlapping bacterial and fungal feature set provided similar diagnostic performance (FIGS. 7C-1 and 7C-2) with substantially fewer taxa, and with putative applicability to other cancer types, the 300 species for additional analyses were then used. It was recognized that WIS-overlapping bacteria previously demonstrated strong associations between smokers and non-smokers, leading us to question whether a difference in smoking histories was potentially driving the observed diagnostic performance, since 82.79% (332/401) of our lung cancer samples had known smoking exposure. Hence, after subsetting healthy and cancer-bearing CODICES subjects to those with known smoking histories, the stacked ML was repeated. Surprisingly, it was found that the diagnostic performance was higher than before among only-smokers (AUROCAll=93.0%; FIGS. 26A-1 and 26A-2), suggesting that smoke-associated pathology potentially permits additional leakage of microbial DNA into circulation, creating a stronger signal for diagnosis in the setting of cancer.

### Classic Metagenomic Analyses of Plasma-Derived Microbiomes

Similarly, the presence of damaged tissue and concomitant vascular barriers among patients with lung cancer led us to hypothesize that alpha diversity would be higher in plasma from cases than controls, even though lung tumor tissue has lower alpha diversity compared to adjacent normal tissue within the same subject. Indeed, after rarefying WIS-overlapping markers, a significantly higher alpha diversity among lung cancer plasma samples compared to healthy controls (FIG. 7D) was observed. Calculating robust Aitchison beta diversity distances confirmed the strength of separation identified in the stacked ML, with a pseudo-F PERMANOVA statistic of 115.92 (p=0.001; FIG. 7E). Microbial differential abundance modeling with ANCOM-BC then demonstrated more lung cancer than healthy-associated biomarkers with two WIS-overlapping bacteria particularly driving strong separation, Pseudomonas oleovorans and Pseudomonas mendocina (FIG. 7F, circled dots). Notably, both of these bacteria had higher relative abundances in lung cancer samples than either healthy samples or blanks (FIG. 7G, top), and substantially greater aggregate genome coverage in plasma samples compared to blanks, with P. oleovorans demonstrating 55.24% coverage in the plasma but just 0.42% coverage among blanks. Thus, classical metagenomic analyses of cancer-associated microbial biomarkers in plasma confirm the diagnostic capacity of cf-mbDNA in a large cohort of treatment-naive individuals.

Next whether CEA and OPN proteomic markers could enhance the diagnostic performance of metagenomes was explored. After verifying their stage-wise concentration increases, with OPN providing better performance in early-stage comparisons than CEA (FIG. 7H), it was found that the combined proteometagenomic classifier increased cancer discrimination to that originally observed when using all putatively decontaminated rep206 features (AUROC=92.1%; FIG. 7C-1, upper left; FIG. 7I) a feature set 21.5-fold greater in size. Moreover, although protein features individually provided only moderate cancer detection with the same stacked ML approach (FIGS. 26B-1 to 26B-4), their performance improved at higher stages, counterbalancing the reversed trend of our microbial classifier. This improvement was also observable when evaluating sensitivity at 99% specificity (FIG. 7J), which in itself demonstrated state-of-the-art sensitivity for detecting stage I lung cancers (˜50% at 99% specificity) compared to existing methods that have evaluated epigenetic or combined ctDNA and proteomic marker panels.

To further validate that this diagnostic performance was not a result of metagenomic read misalignments, the short V6 region of 16S rRNA in a subset of 335 plasma samples processed on a single sequencing run with 16 extraction blanks and four positive control mock communities, including samples from 142 lung cancer-bearing patients and 96 healthy subjects was then amplified. After processing sequenced amplicons with Deblur for taxonomic compositions and decontaminating, as well as PICRUSt2 identification, their abundances were then inputted into the stacked ML pipeline. Remarkably, both amplicon-based taxonomic compositions and functional pathways strongly distinguished lung cancers from healthy controls (AUROC range: 83.4-86.3%; FIG. 7K) and provided similar diagnostic performance to shotgun metagenomes when combined with proteomic information (AUROC: 93.6%). While simultaneously confirming the validity of the shotgun metagenomic findings, this additionally demonstrates (i) the existence and utility of plasma-derived amplicon features for cancer diagnosis and (ii) the applicability of functional microbial biomarkers for cancer diagnosis. The proteo-amplicon-based strategy would further be highly cost efficient, utilize minimal plasma volumes, and provide strong diagnostic performances.

### Discriminating Lung Cancer Against Risk-Matched, Diseased Controls

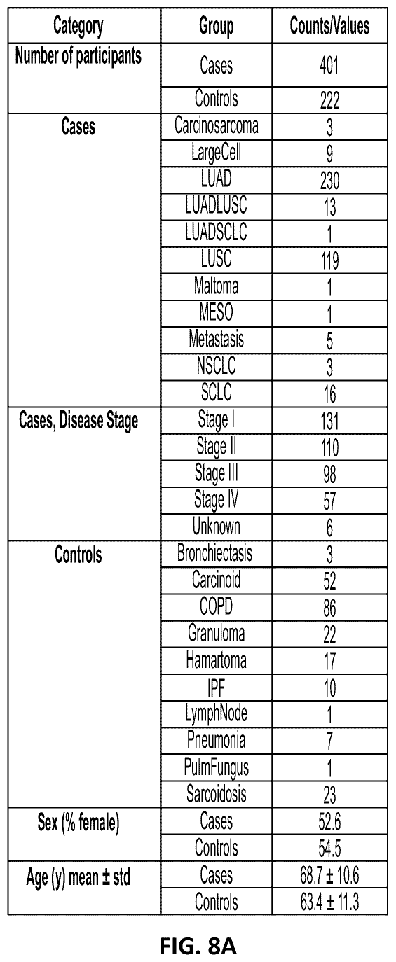

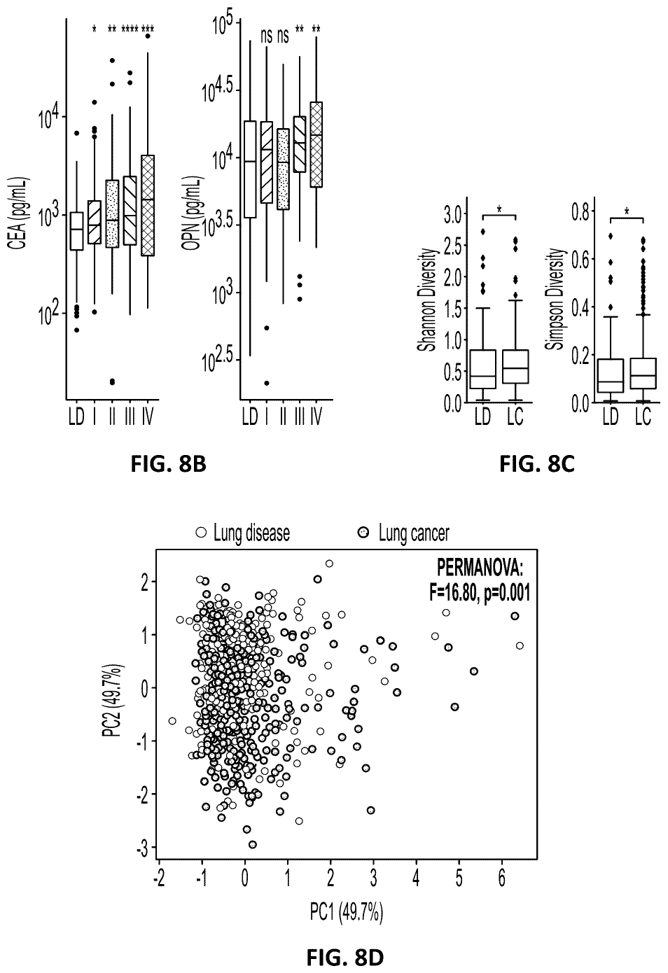

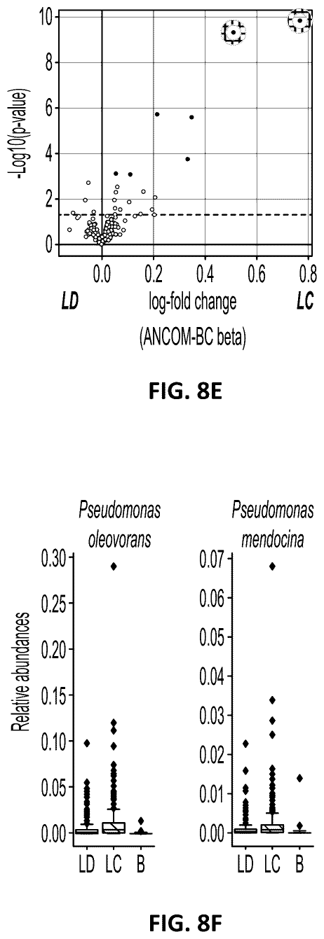

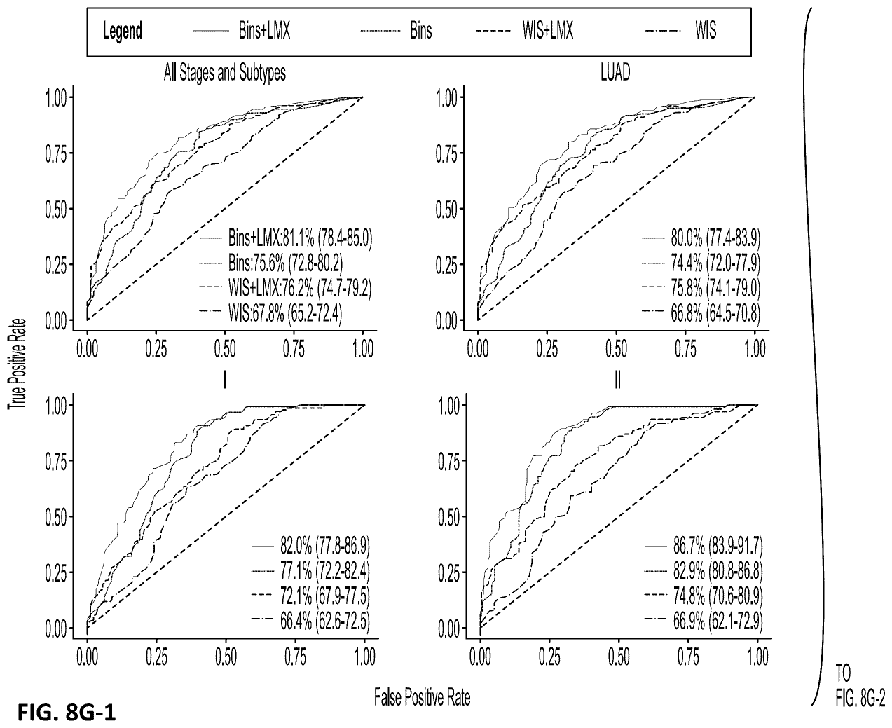

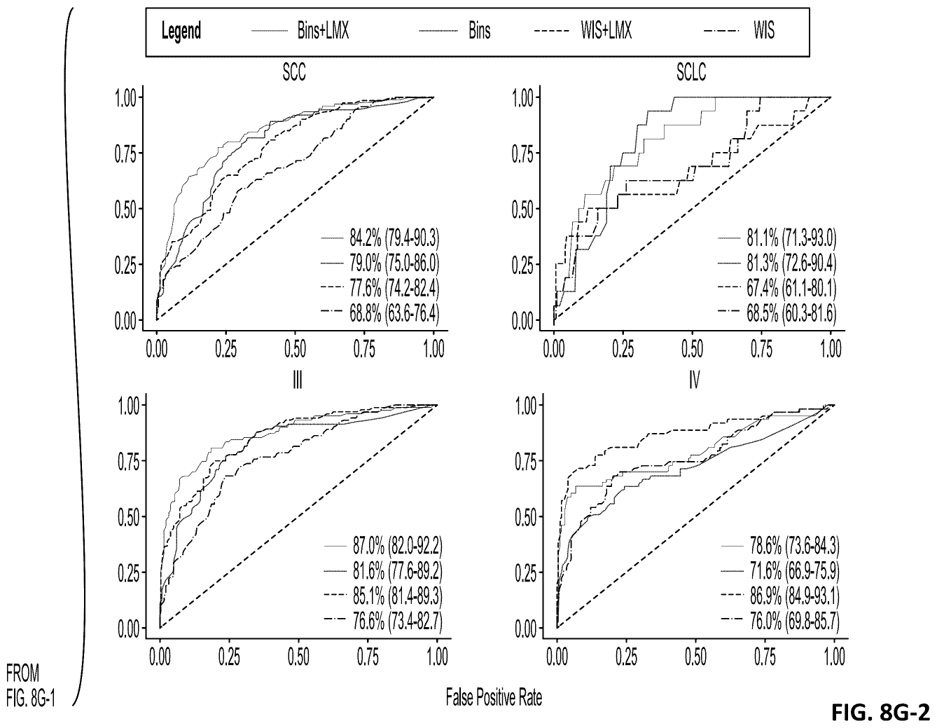

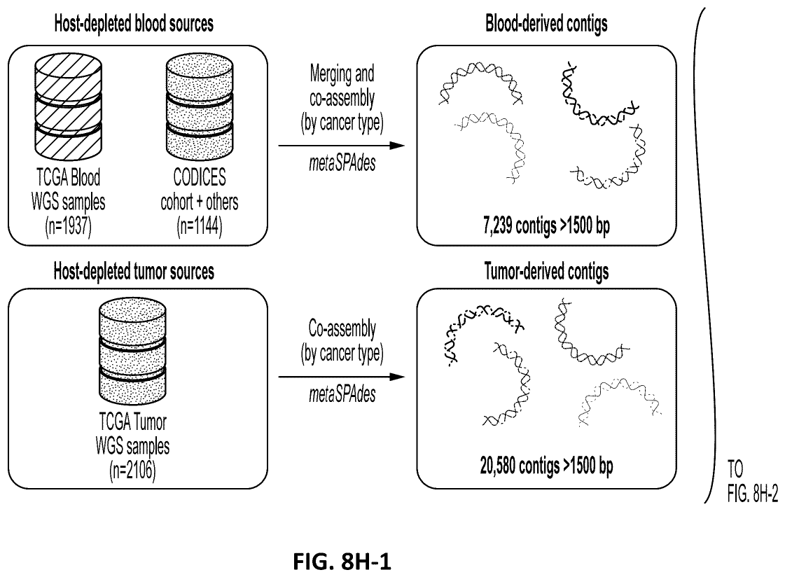

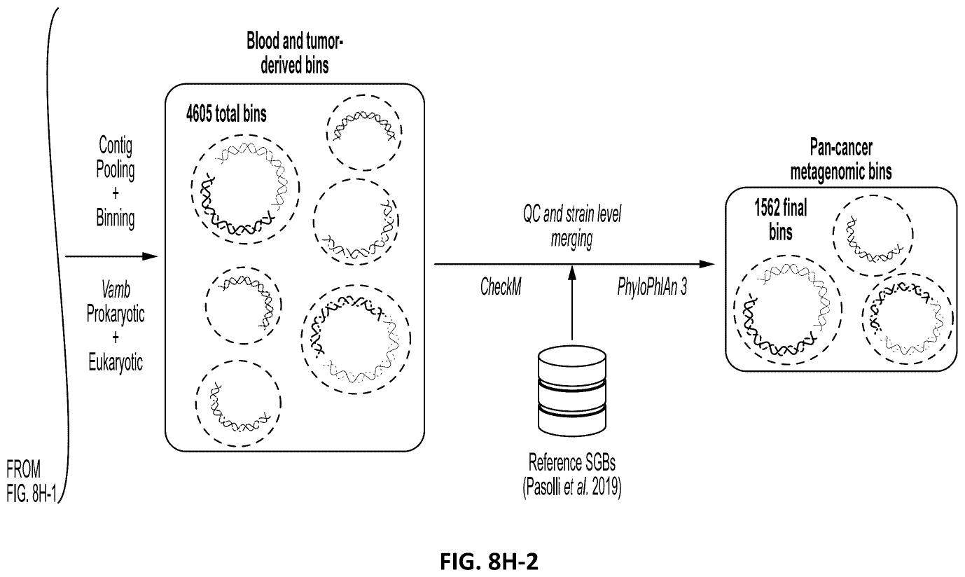

Nonetheless, with perhaps the exception of multi-cancer tests, it is currently rare for lung cancer screening to be conducted in low-risk settings. Thus, next the diagnostic performance of the same lung cancer (LC) patients against 222 age-, sex-, and smoking history-matched (n=161 smokers, or 72.5%) adults with lung diseases (LDs) of diverse etiologies (FIG. 8A) was evaluated, as recommended by other studies. First plasma-derived CEA and OPN again had stepwise, stage-specific, significant increases compared in LCs relative to LDs (FIG. 8B) was verified, followed by classic metagenomic analyses of WIS-overlapping bacterial and fungal features (FIGS. 8C-8F). Notably, the separation of LC and LD samples was weaker albeit still statistically significant compared to LC versus healthy samples for every comparison: more similar alpha diversities (FIG. 8C), a weaker pseudo-F PERMANOVA statistic with robust Aitchison distances (F=16.80, p=0.001; FIG. 8D), and fewer differentially abundant features (FIG. 8E). This change was also noticeable on the lung cancer-associated P. oleovorans and P. mendocina, with an increase in LD relative abundances compared to healthy controls in the direction of LC samples (FIGS. 8E-8F).

Analogously, stacked ML of LC versus LD of WIS-overlapping microbes provided worse discrimination than its LC versus healthy counterpart, even when adding CEA and OPN to the analysis (AUROC=76.2%; FIGS. 8G-1 and 8G-2). Moreover, when subsetting predictions across histological subtypes and stages, stage IV detection was the only comparison with similar performance as in LC versus healthy samples (FIG. 8G-2, lower right; FIG. 7C-2, lower right). Repeating the amplicon-based 16S rRNA approach between the same 142 LC and new 97 LD samples replicated the reduction in performance (AUROCAll=70.8%; FIGS. 26C-1 and 26C-2). These analyses suggested the limitations of using known cancer-associated microbial biomarkers, particularly in the context of risk-matched controls, which have been infrequently employed in large cancer or cancer microbiome studies. They also suggest that proteo-amplicon-based strategies may not provide sufficient diagnostic performance unless additional features can be added to the test.

Since WIS-overlapping bacteria and fungi were identified outside the context of risk-matched controls and further did not examine plasma samples, which are known to contain metagenomic features that are poorly-represented in existing microbial databases, de novo metagenome assembly using all blood and primary tumor whole genome sequenced (WGS) samples from The Cancer Genome Atlas (TCGA) alongside samples from the CODICES cohort (FIGS. 8H-1 and 8H-2) was then performed. Specifically, blood samples from all 25 available TCGA cancer types (n=1937 samples) with matched cancer types in the plasma-derived CODICES cohort while separating LD, healthy, and blank samples into distinct groups, followed by metagenomic co-assemblies with metaSPAdes was merged. In parallel, metagenomic co-assemblies on 24 TCGA primary tumor types (n=2106 samples) were also performed. Collectively, these analyses identified 27,819 contigs >1500 bp in length, which were subsequently pooled and binned using a deep variational autoencoder. These 4605 metagenomic bins were quality controlled with CheckM and then merged at the predicted strain level using PhyloPhlAn 3 and a large database of human-associated species-level genome bins (SGBs), resulting in 1562 final, pan-cancer, pan-tissue, and blood, metagenomic bins (FIGS. 8H-1 and 8H-2).

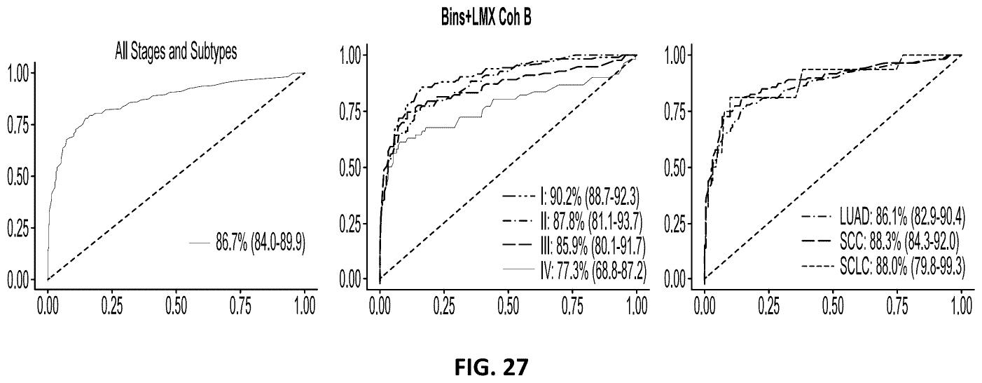

Remarkably, the metagenomic bins outperformed WIS-overlapping, cancer-associated features to distinguish LC from LD samples in every comparison other than stage IV disease (FIG. 8G-2). Combining the metagenomic bins with CEA and OPN further synergistically increased diagnostic performance (FIGS. 8G-1 and 8G-2, ‘Bins+LMX’). Reapplication of the de novo metagenomes paired with CEA and OPN to the low-risk analysis revealed similar performances, particularly in early-stage disease (Stage I AUROC: 90.2%; FIG. 27), although the performance gain was most notable in the high-risk setting. Beyond better AUROC values, the shape of the bin-based ROC curve in clinical stage I and II disease (FIG. 8G-1, lower left and lower right) also suggested higher specificities at high fixed sensitivity cutoffs (e.g., 97% sensitivity), suggesting the metagenomic bins may be particularly useful for ruling out malignant disease while mitigating false positives.

### Evaluation of De Novo Assembled Metagenomic Bins in TCGA

Having created a novel set of metagenome-assembled bins with superior diagnostic performance for identifying LDs in the CODICES cohort, their cancer type specificity and generalizability in two independent cohorts, comprising 16,049 total samples across 35 conditions (34 cancer types and healthy), including in samples that were independent from the metagenome assembly was then validated.

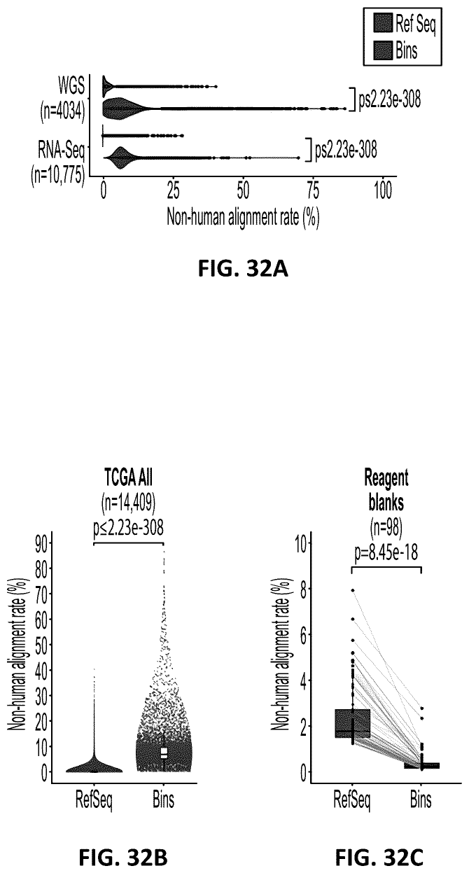

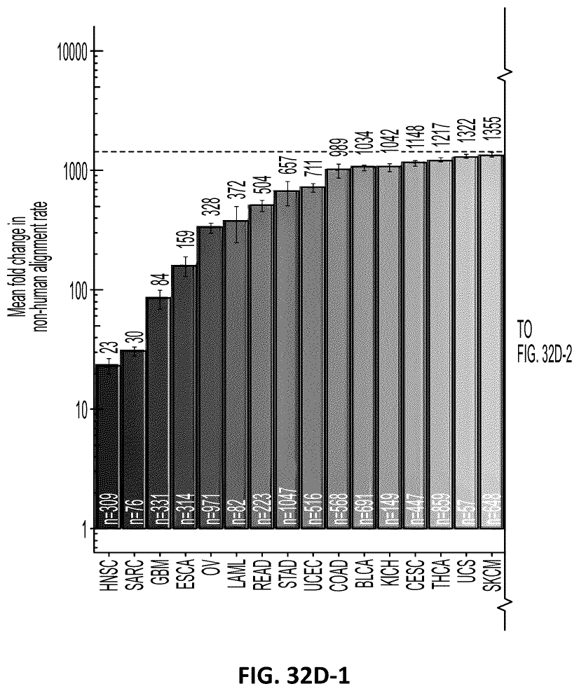

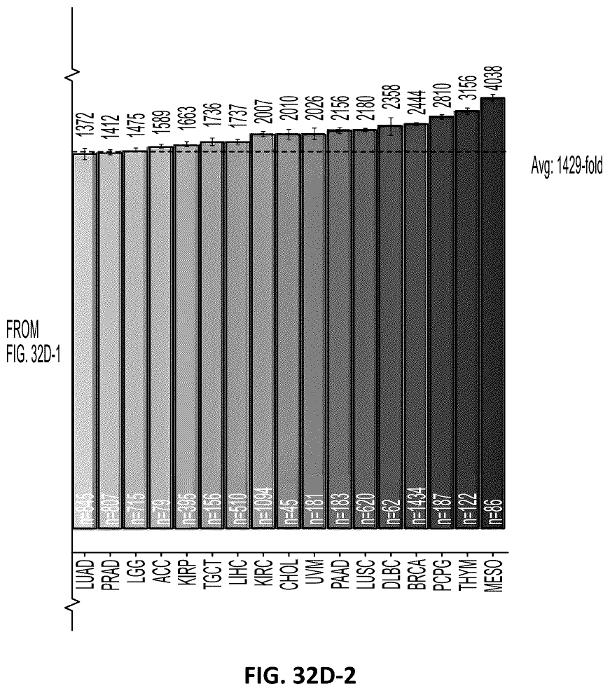

First all 15,512 base-quality filtered, doubly host-depleted, length-restricted (>45 bp), whole-genome sequenced (WGS) and RNA sequenced (RNA-Seq) samples in TCGA against the metagenomic bins were re-aligned. A total of 14,809 samples (95.47%) had non-zero alignments against 1545 of the bins, with normal adjacent tissues (NATs), which were not part of the metagenome assembly, comprising 73.26% (515/703) of the dropped-out samples. Remarkably, the paired alignment rate of non-human reads across all cancers, sample types, and experimental strategies increased by a median of 891-fold to the bins compared to RefSeq (Wilcoxon signed-rank: p≤2.23×10⁻³⁰⁸; FIG. 32B). To confirm that higher alignment rates were not caused by mapping more contamination, and since TCGA did not include sequencing blanks, non-human reads derived from sequencing 98 reagent-only blanks—collected across all CODICES and NYU cohorts' sequencing runs were mapped to RefSeq and the bins, finding that the bins significantly reduced contaminant mapping (Wilcoxon signed-rank: p=8.45×10⁻¹⁸; FIG. 32C). Critically, the bins provided significantly higher non-human mapping rates in TCGA samples than blanks (Wilcoxon: p=6.63×10⁻⁶⁴), with 99.8% of TCGA samples exceeding the blanks' median rate of mapping to the bins (0.26%). These observations were consistent when stratifying TCGA samples by WGS or RNA-Seq. Thus, compared to RefSeq, the bins dramatically increased biological microbial mapping rates while mitigating reagent-based contaminant mapping, despite using 7.65-fold fewer genomic features.

Bin alignment rates by experimental strategy revealed significant increases (Wilcoxon signed-rank: p≤2.23×10-308; FIG. 32A), despite RNA-Seq samples being excluded from the metagenome assembly; moreover, the bins reconciled WGS (95% CI: [8.25, 8.90]%) and RNA-Seq (95% CI: [8.42, 8.61]%) alignment rates, which were disparate in RefSeq (WGS, 95% CI: [1.66, 1.88]%; RNA-Seq, 95% CI: [0.32, 0.39]%). Calculating mean fold changes per cancer type between bins and RefSeq revealed an average 1429-fold increase in non-human mapping rates (FIGS. 32D-1 and 32D-2), with lung adenocarcinoma (LUAD) and lung squamous cell carcinoma (LUSC) increasing 1372-fold and 2180-fold, respectively.

Quality control of metadata (e.g., removing sequencing centers with a single cancer type), and subsetting to a single sequencing platform accounting for 98.00% of the samples, left 14,454 tissue and blood samples (93.18% of total) for downstream analyses.

Although batch effects were still persistent in TCGA metagenomic bin abundances, they were mitigated (FIGS. 12A-B). For example, whereas our original principal variance components analysis (PVCA) of raw, Kraken-derived genera abundances in TCGA revealed that approximately 34% and 36% of the data variance were attributable to sequencing center and experimental strategy, respectively, they were reduced to 20.3% and 3.2% variance, respectively, in the metagenomic bin data. Moreover, when separately analyzing raw WGS and RNA-Seq samples, the disease type-associated variance among WGS samples—those used for metagenomic assembly—exceeded that from other technical variables without any batch correction (FIGS. 12C and D), suggesting that metagenomic assembly may be able to sufficiently mitigate technical batch effects in retrospectively analyzed cancer microbiome datasets.

Nonetheless, batch correction was initially performed to utilize the full TCGA cohort, computing 10-fold cross-validation ML using gradient boosting in a one-cancer-versus-all-others manner to test cancer type specificity (FIGS. 13A-C). This analysis revealed pan-cancer discrimination among 32 primary tumor types (AUROCavg=84.31%, 95% CI: [83.28, 85.34]%); moderate discrimination of tumors versus NATs (AUROCavg=73.14%, 95% CI: [70.56, 75.71]%), potentially due to NAT sample dropout; and very strong performances discriminating among blood samples from 20 cancer types (AUROCavg=96.21%, 95% CI: [95.62, 96.80]%). Importantly, using the metagenomic bins, lung adenocarcinoma-derived blood samples had the highest AUROC and AUPR combination performance in TCGA (AUROCavg=97.92; AUPRavg=89.99%), which was not the case in either our bacterial or fungal-centric analyses, suggesting that the co-assembly with samples from the CODICES cohort improved concomitant cancer type specificity.

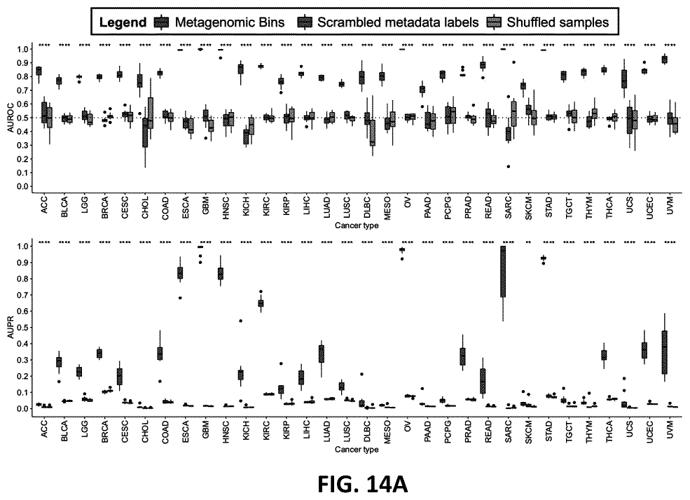

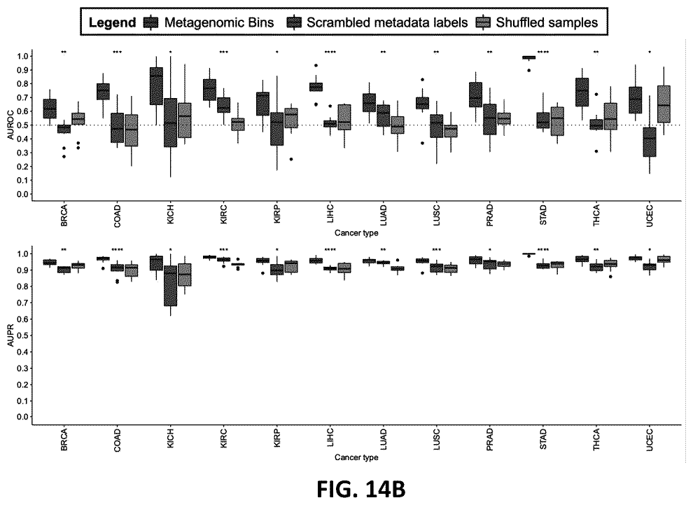

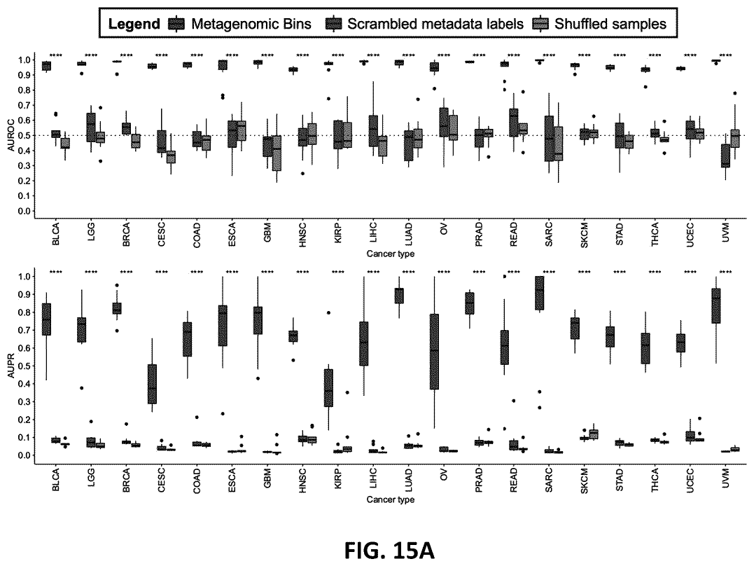

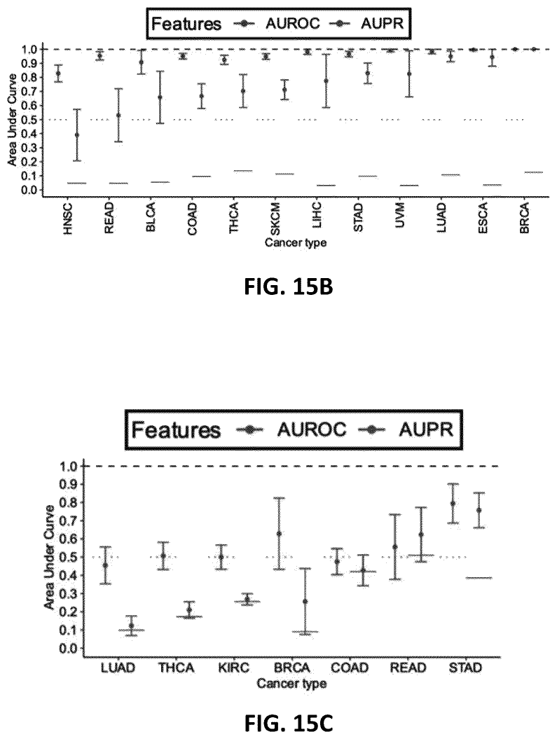

As negative controls, all ML analyses using scrambled metadata or shuffled count data on matched CV folds, followed by comparing the expected null to actual performances, finding statistically significant better cancer type discrimination than null models in all cases (FIGS. 14A-B and FIG. 15A) was then repeated. Moreover, when subsetting to all TCGA blood samples in patients with early-stage (Ia-IIc) cancer, followed by repeating the ML, a found strong pan-cancer discrimination (AUROCavg=95.23%, 95% CI: [94.05, 96.42]%), including specifically for lung adenocarcinoma (AUROCavg=98.31; AUPRavg=94.90e %; FIG. 15B) was found. Similar performances in early and late-stage cancer suggest that it may be difficult to distinguish stage I versus stage IV tumors using metagenomic bins, which indeed was the case (FIG. 15C).

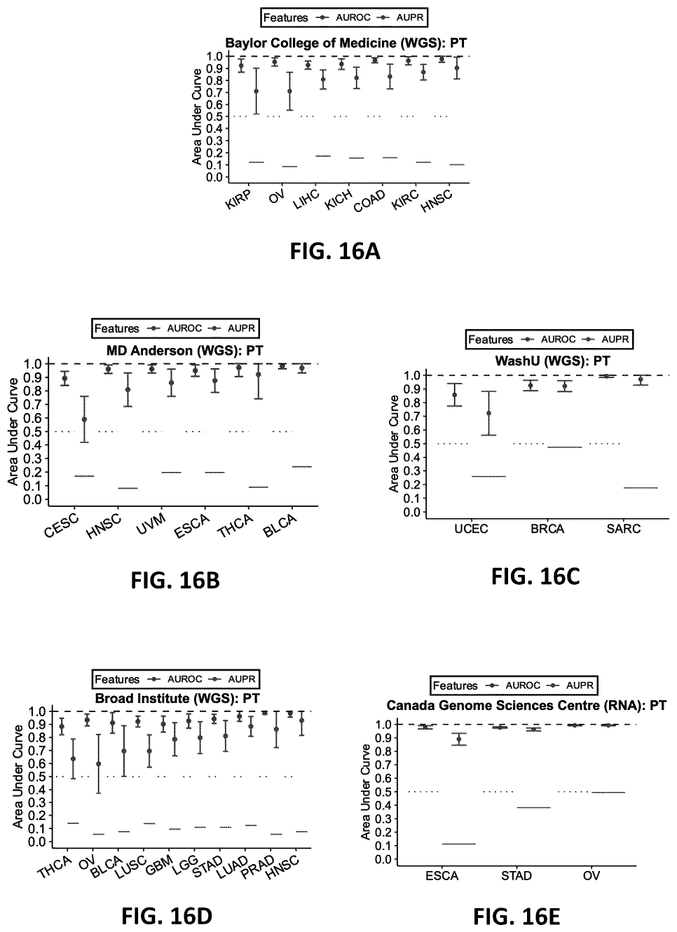

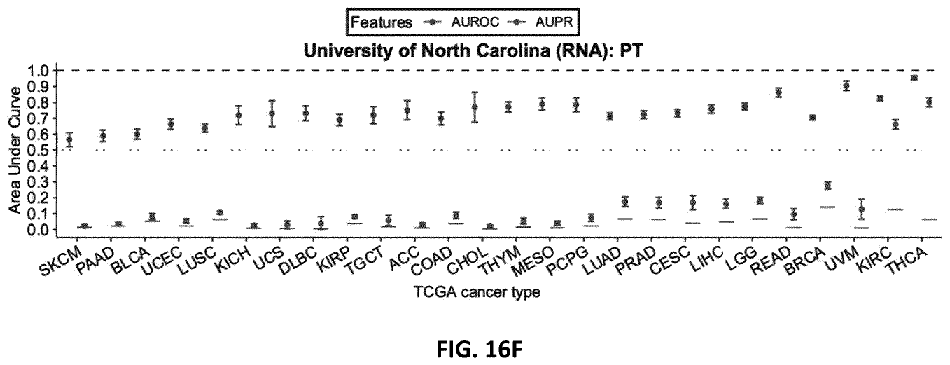

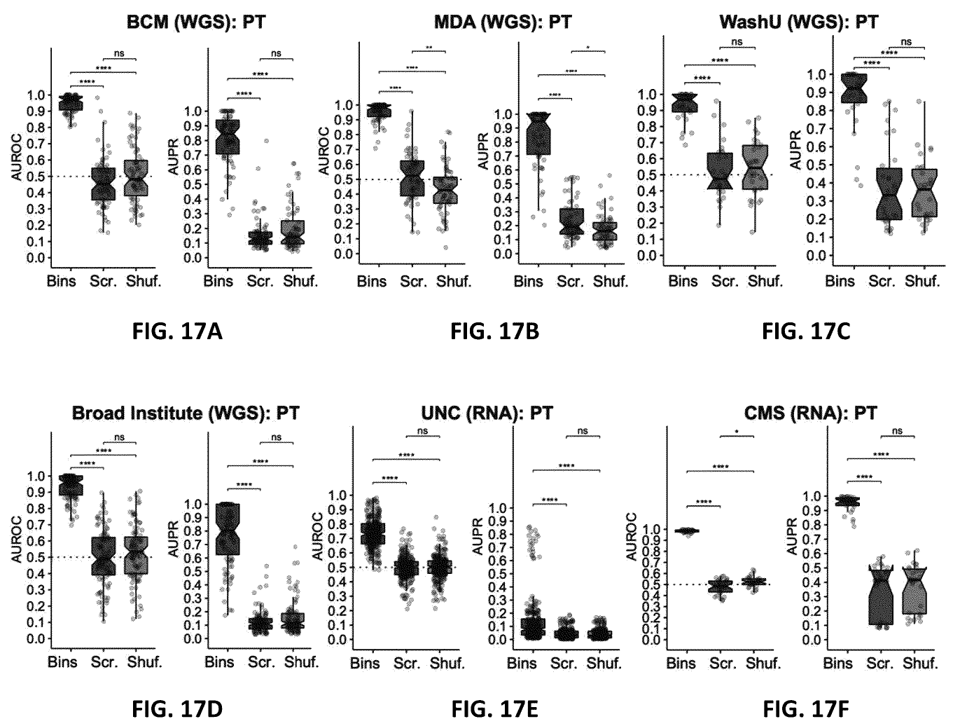

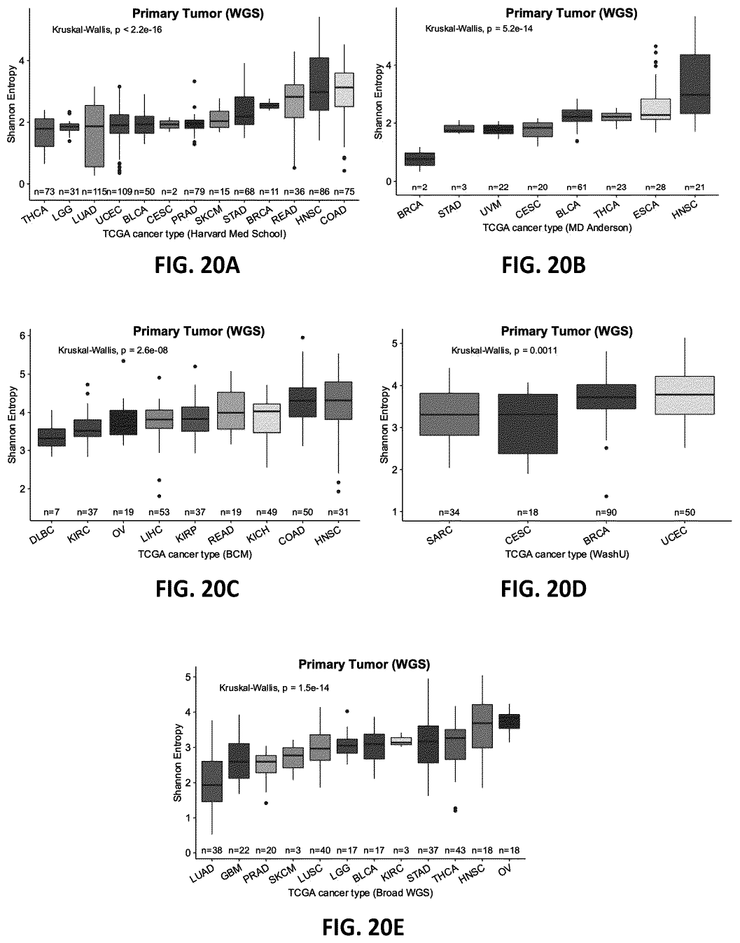

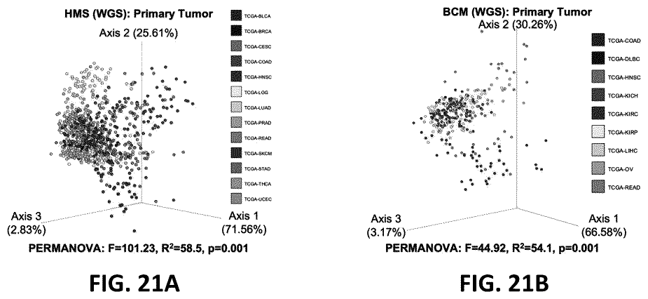

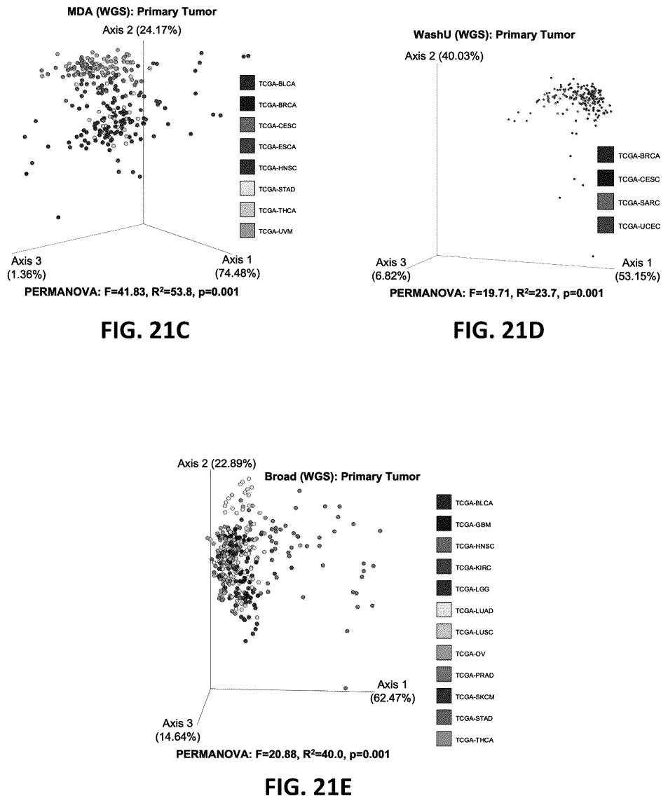

Next, to avoid batch correction altogether, all raw, metagenomic bin abundances to individual sequencing centers, experimental strategies (WGS or RNA-Seq), and sequencing platform (Illumina HiSeq) prior to evaluating 10-fold ML were subsetted. This provided strong tumor type discrimination in Harvard Medical School (HMS) samples (FIG. 9A, AUROCavg=98.27%, AUPRavg=89.56%) and every other sequencing center (FIGS. 16A-16F). Repeating scrambled metadata and shuffled count data control analyses using raw data again revealed significantly better-than-null, pan-cancer, tumor type discrimination in every subset (FIG. 9B; FIGS. 17A-17F). Applying classic metagenomic analyses further revealed significant, cancer type-specific alpha and beta diversity distribution in every testable primary tumor subset, oftentimes showing that cancer type explained more than half of the data variance in metagenomic bin abundances (FIG. 9C; FIGS. 20A-20E; and FIGS. 21A-21E). To further substantiate cancer type specificity of the bins, applied differential abundance modeling using ANCOM-BC was applied to all raw data subsets, again finding differentially abundant bins in every comparison (FIGS. 9D and 9H; representative examples in FIGS. 22A-22G).

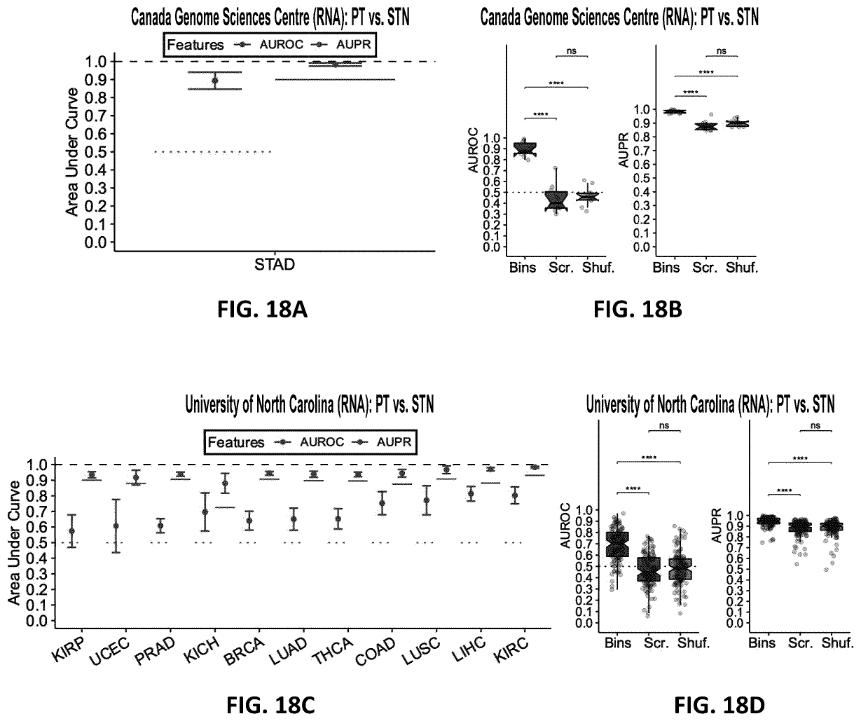

Repeating tumor versus normal ML analyses using raw metagenomic bin subsets confirmed 9 of 12 tumor types, although aggregated comparisons were still significantly better than null models (FIGS. 18A-18D).

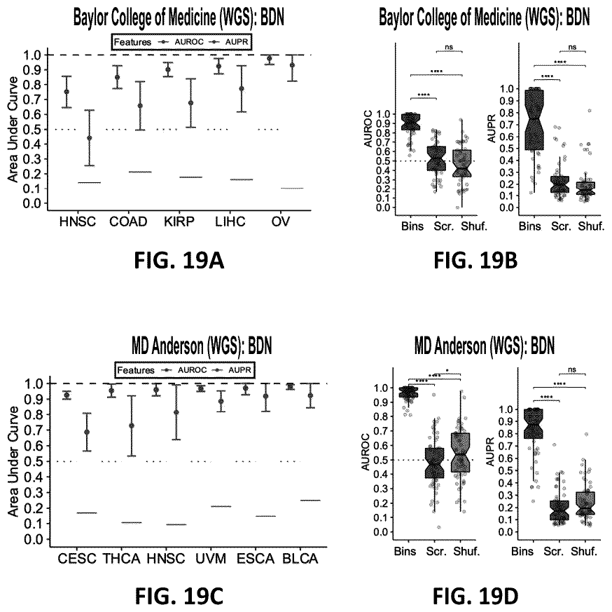

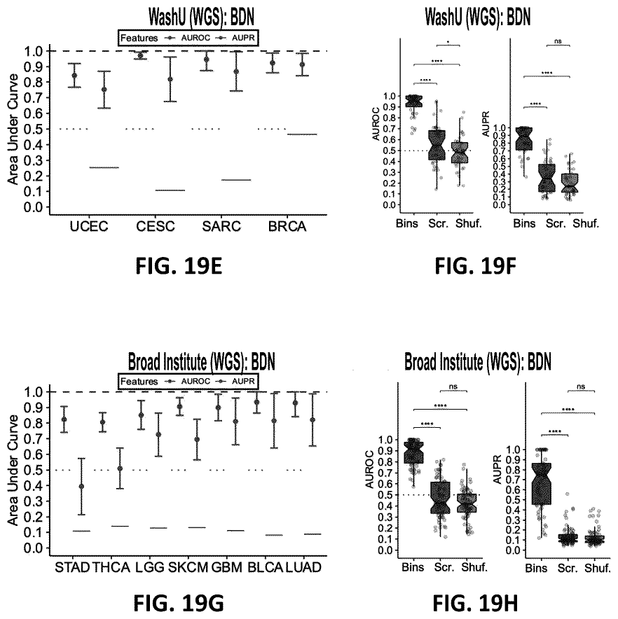

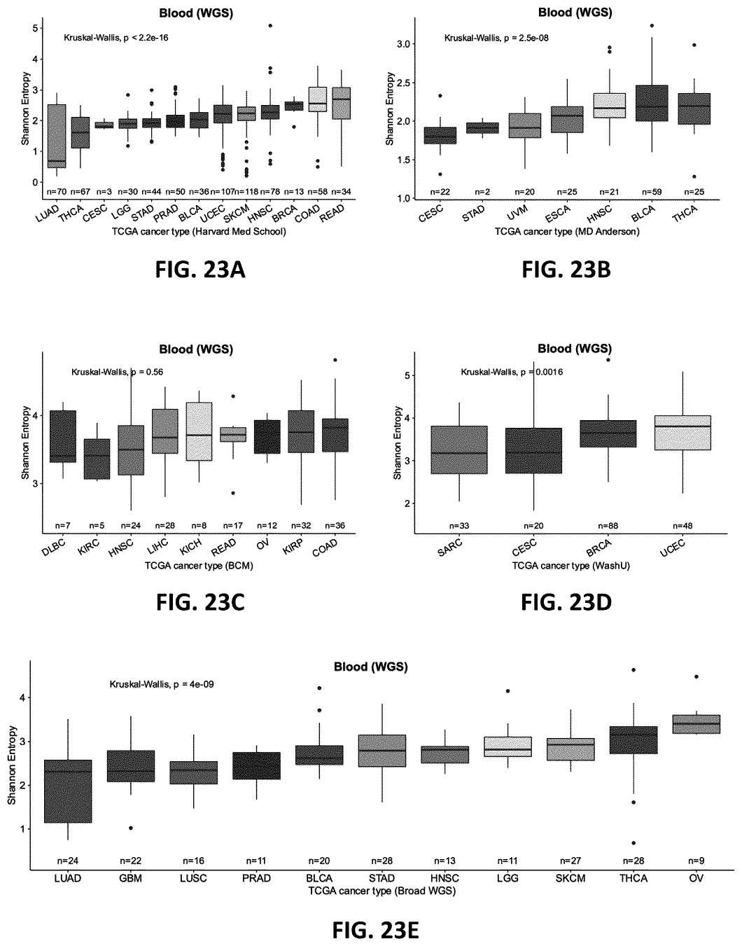

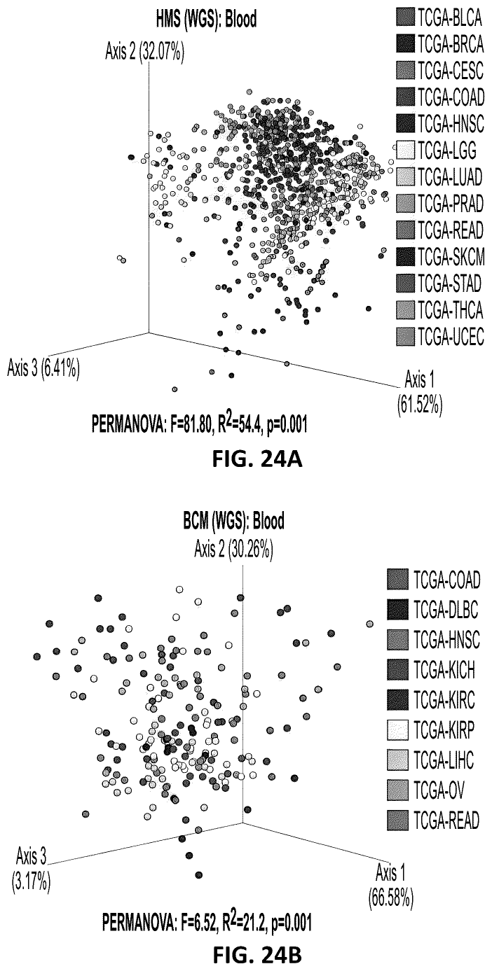

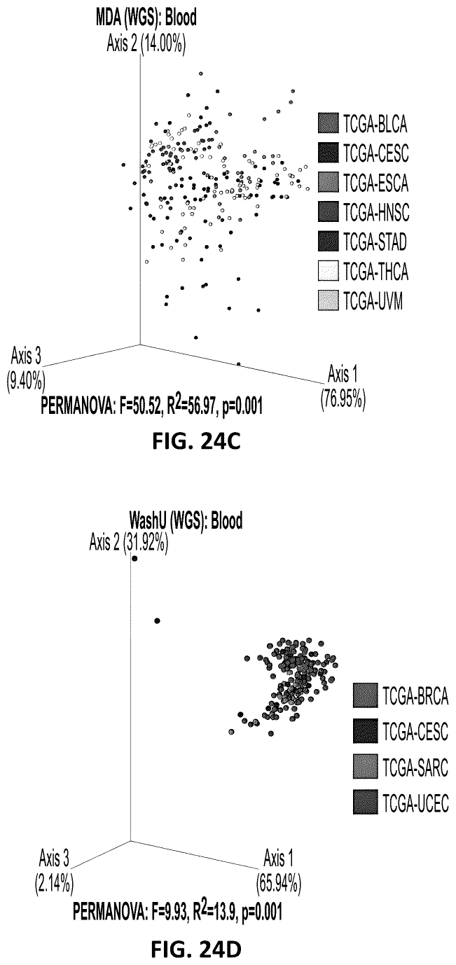

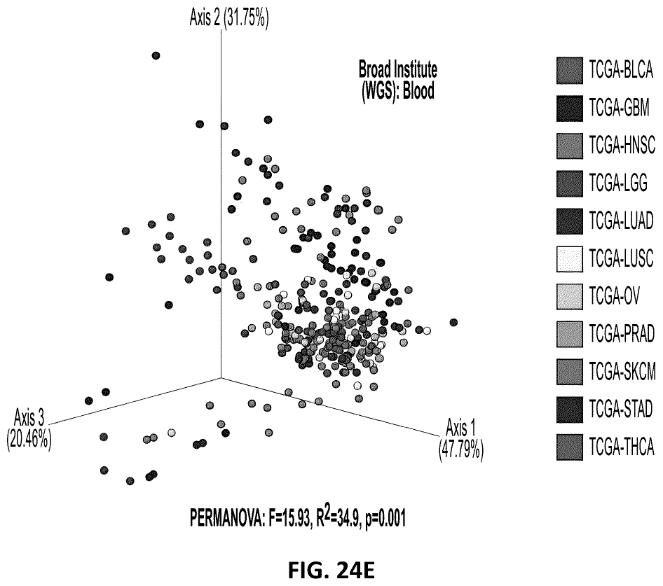

All blood-based cancer type analyses using one-versus-all-others ML (FIG. 9E; FIGS. 19A, 19C, 19E, and 19G), negative control ML analyses (FIG. 9F; and FIGS. 19B, 19D, 19F, and 19H), alpha diversity (FIGS. 23A-23E), beta diversity (FIGS. 24A-24E), and differential abundance modeling (FIGS. 25A-25E) was then replicated. In all of these analyses, with the sole exception of alpha diversity in a single sequencing center subset (Baylor College of Medicine, FIG. 23C), the metagenomic bin-based cancer type variation among TCGA blood samples was significant. Furthermore, the two sequencing centers containing lung adenocarcinoma-derived blood samples (HMS, Broad Institute) consistently showed it had the strongest diagnostic performance among every other cancer type (FIG. 9E; and FIG. 19G), again suggesting that CODICES cohort co-assembly improved lung cancer detection.

Additionally, since one-versus-all-others comparisons can inflate the no information rate (NIR), multiclass ML, wherein all cancer types are simultaneously considered by the gradient boosting algorithm was performed. Applying this to all 32 primary tumor types in batch-corrected data replicated the one-versus-all-others performance (avg. pairwise AUROC=83.19%; FIG. 13D), with a mean balanced accuracy of 65.17% in comparison to a 10.48% NIR (p<2.2×10-308). Blood-based, multiclass ML among 24 cancer types with batch-corrected data performed even better (avg. pairwise AUROC=95.50%; FIGS. 9I-1 and 9I-2), with a mean balanced accuracy of 79.26% in comparison to an 8.92% NIR (p<2.2×10-308). Since all TCGA blood samples were WGS, and since raw WGS samples had less sequencing center variation than disease type using PVCA, multiclass blood ML using raw metagenomic bin abundances, with nearly identical results (avg. pairwise AUROC=95.14%; mean balanced accuracy=79.02%; FIG. 13E) was also tested. Comprehensively, the metagenomic bins are cancer type specific.

### Evaluating De Novo Assembled Metagenomic Bins as Database for Cancer-Associated Microbial Features for Non-Blood Derived Microbial Reads: Fecal Microbiome Data

Having demonstrated that the metagenomic bins are cancer type specific when examined against TCGA tissue and blood samples, the bins were then analyzed to determine if they could serve as a database of cancer-associated microbial genomes against which sequencing reads from non-blood sources could be aligned. Toward this end, it was hypothesized that the bins may provide diagnostic utility for colorectal cancer (CRC). geographically-disparate, fecal metagenomic CRC cohorts (PMIDs: 25432777, 26408641) from France (FR) and China (CN) were then processed and cross-compared in a subsequent meta-analysis (PMID: 30936547), providing foreknowledge of internal cross-validation and external cohort validation performances.

Beta diversity revealed significant presence-absence differences in FR and CN cohorts between CRC-bearing and healthy subjects (Jaccard PERMANOVAs: p=0.001), but variable effect sizes with Aitchison-based measures and no significant changes in Shannon alpha diversity, as reported (PMIDs: 25432777, 26408641). Nonetheless, application of LOOCV ML on each cohort revealed high AUROCs (FR: 88.77%, CN: 85.64%) that exceeded those published by the original authors (FR38: 84%; CN37: 83.61%) or the meta-analysis (PMID: 30936547; FR: 85%; CN: 81%). Remarkably, without batch correction, cross-application of these models revealed improved performance over LOOCV (FR-to-CN: 88.73%; CN-to-FR: 90.75%), surpassing the international meta-analysis up to 9% AUC. Locking cutoffs using internal LOOCV yielded specificities and sensitivities, respectively, up to 82.0% and 79.2%. Since the FR cohort provided stage information, LOOCV and CN-cross-tested predictions were subsetted to early (stage I-II) and late (stage III-IV) samples, finding equivalently strong early-stage performance (92.03-92.62%). Calculating bootstrapped sensitivities at 92% specificity (PMID: 25432777) showed ≥15-point increases and consistency between LOOCV and CN-cross-testing. Although the CN cohort had lower sensitivity at 92% specificity, its LOOCV and FR-cross-tested values were similar. Thus, the bins apply across diverse sample types (i.e., tissue, blood, and stool), an independent cancer type, and geographically-disparate cohorts while improving diagnosis.

Fecal-derived microbiota changes can also inform distal lung cancer diagnosis. In the absence of publicly-available, fecal shotgun data of lung cancer versus controls, 16S rRNA gene amplicon data from Lim et al. (PMID: 34586729) was explored to determine whether it would be compatible with the metagenomic bins (“Lung-gut” cohort). Despite limited 16S rRNA alignments, significant presence-absence and Aitchison-based beta diversity differences existed, and significant decreases in Shannon alpha diversity not previously reported. LOOCV ML was then performed, matching the CRC cohorts' methods, finding an AUROC of 86.52%, or 10% AUC higher than reported (PMID: 34586729). Remarkably, the 51.6% sensitivity at 92% specificity approached CRC results and equaled the CN cohort's LOOCV results. Unfortunately, healthy saliva samples from the same cohort were not publicly available to compare (PMID: 34586729). Nevertheless, the metagenomic bins are broadly generalizable for improving multi-cancer diagnostic performances, with the data here demonstrating that alignment of sequencing reads from fecal samples (shotgun metagenomic reads and 16S targeted amplicon sequencing) to the pan-cancer metagenomic bins of the present invention, enables the development of colorectal and lung cancer-specific diagnostic classifiers.

Complementarity of Metagenomic Bins with Fragmentomic Information

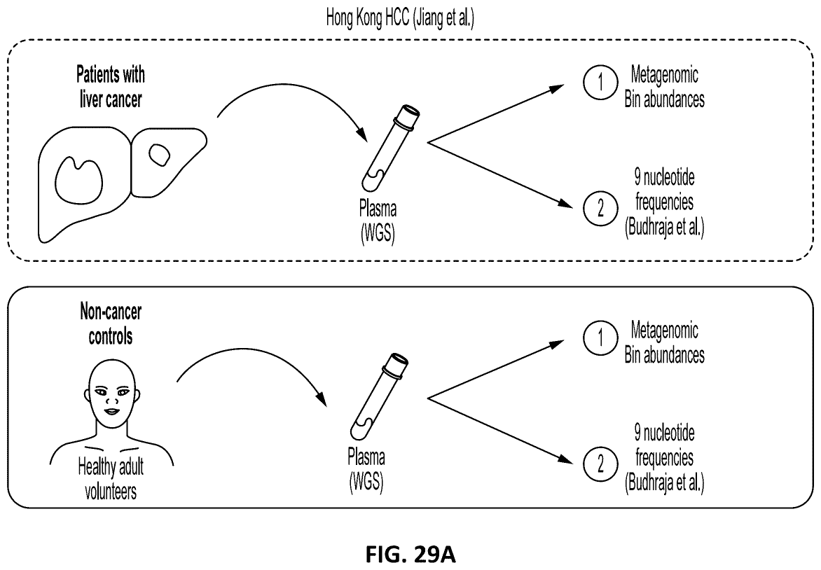

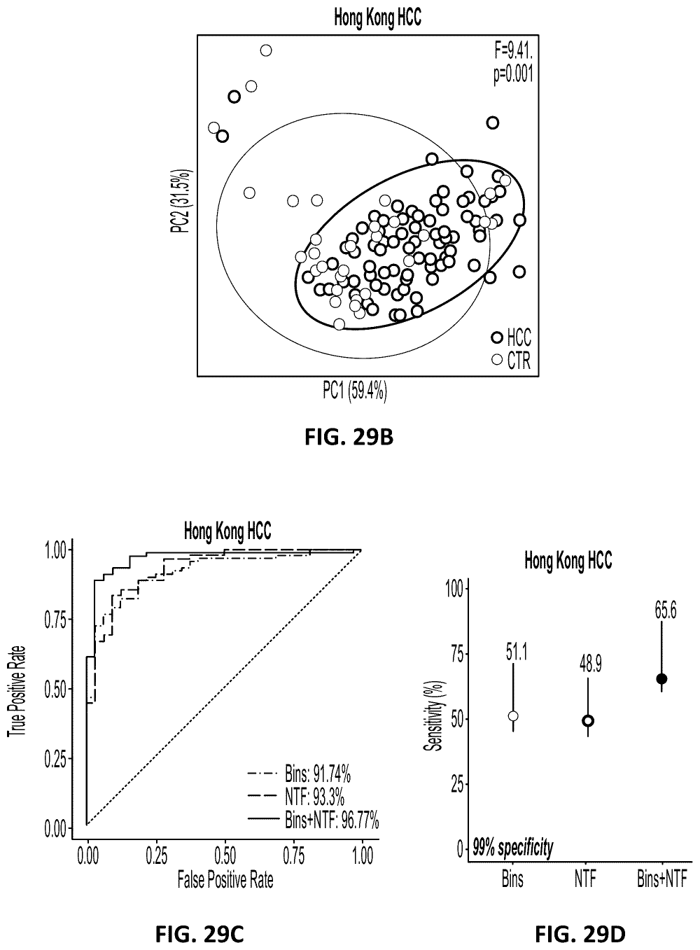

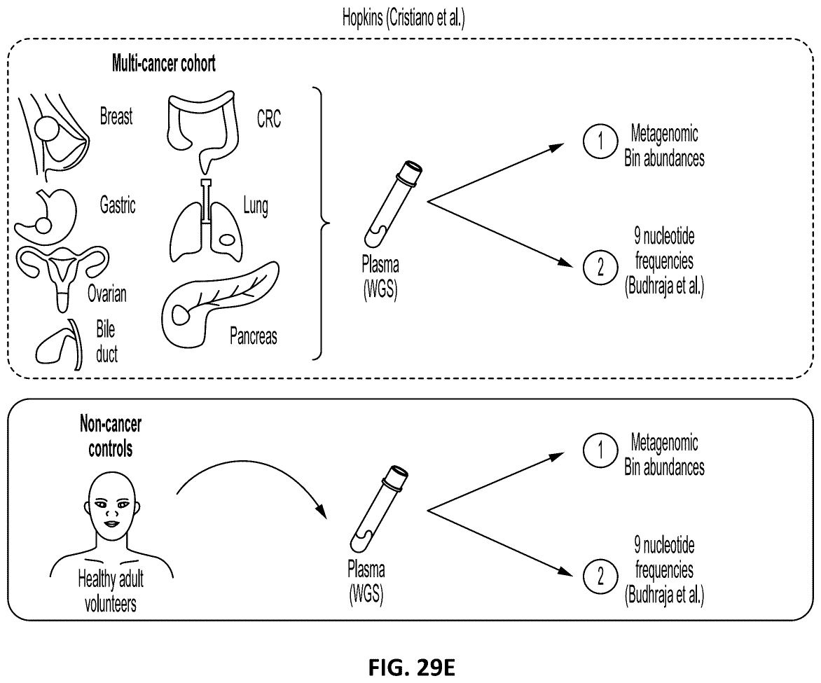

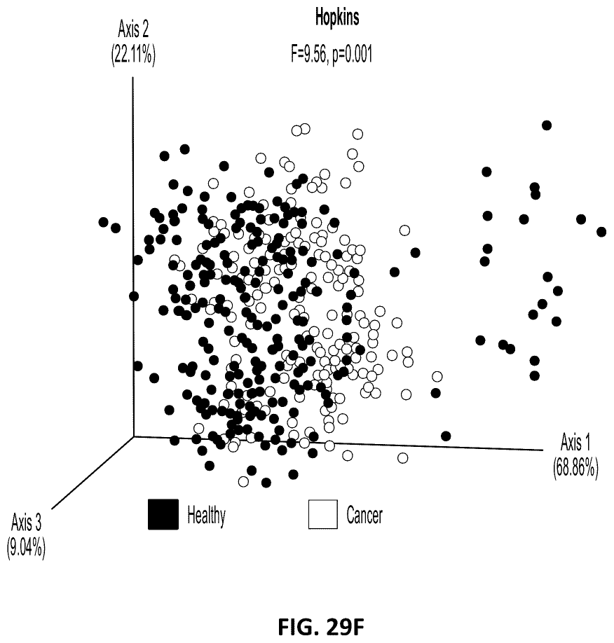

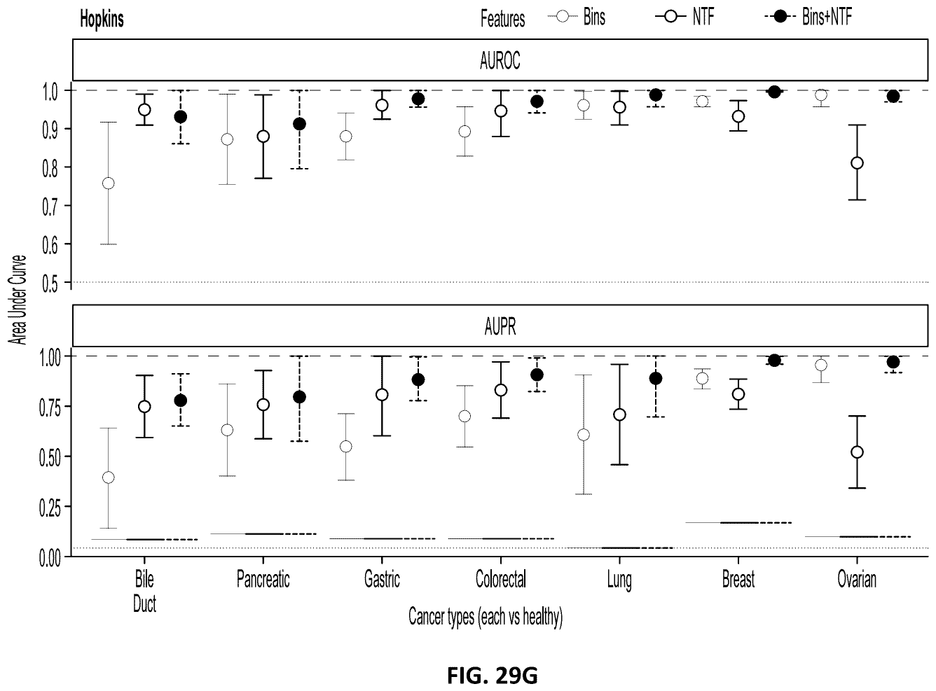

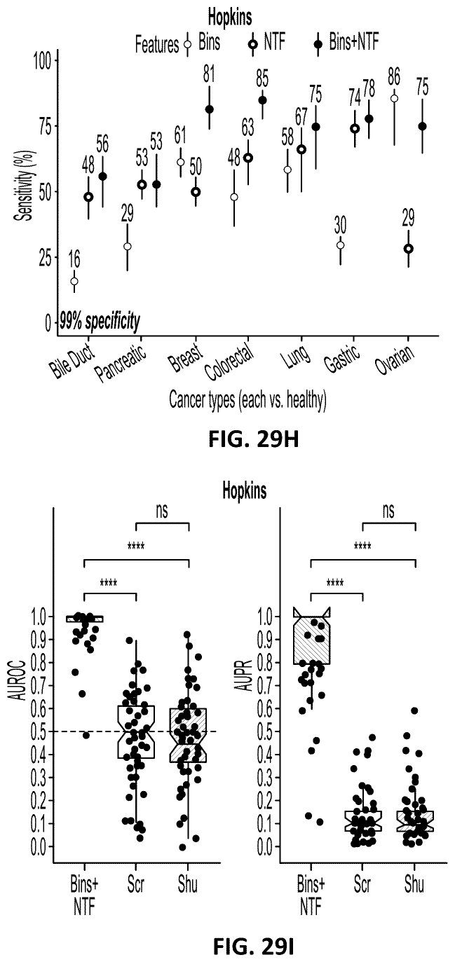

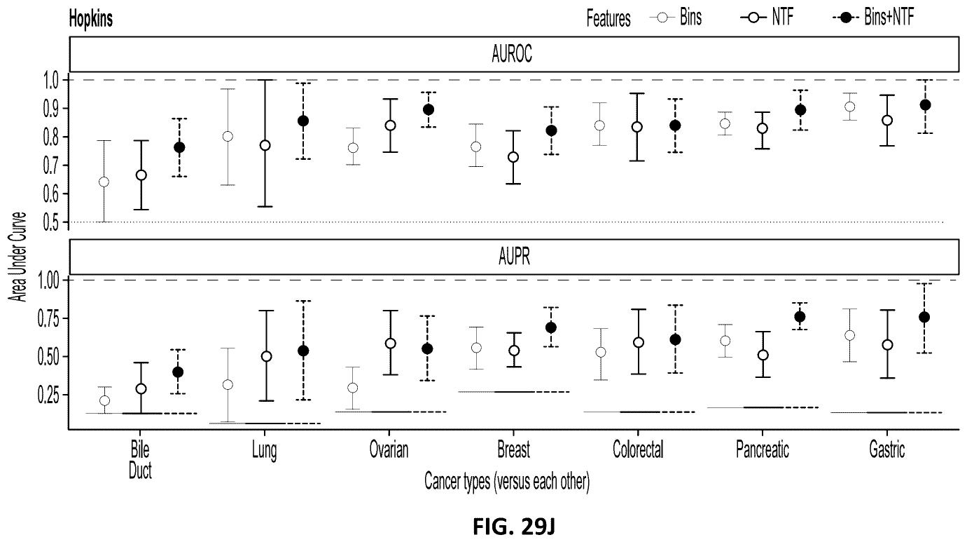

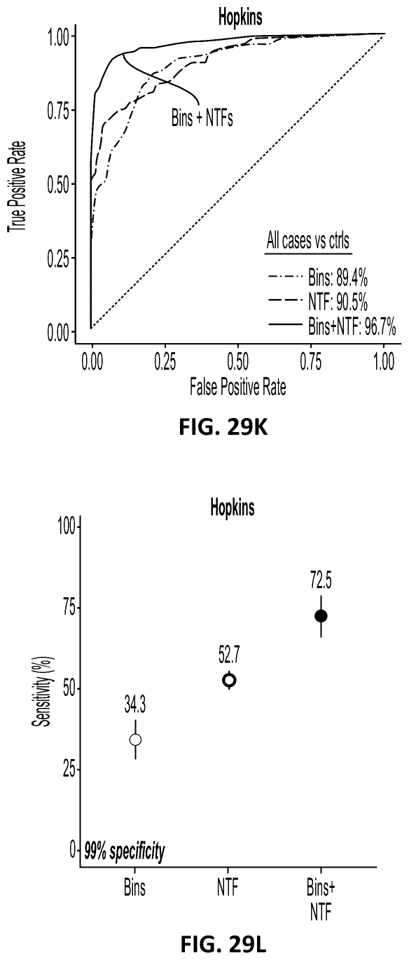

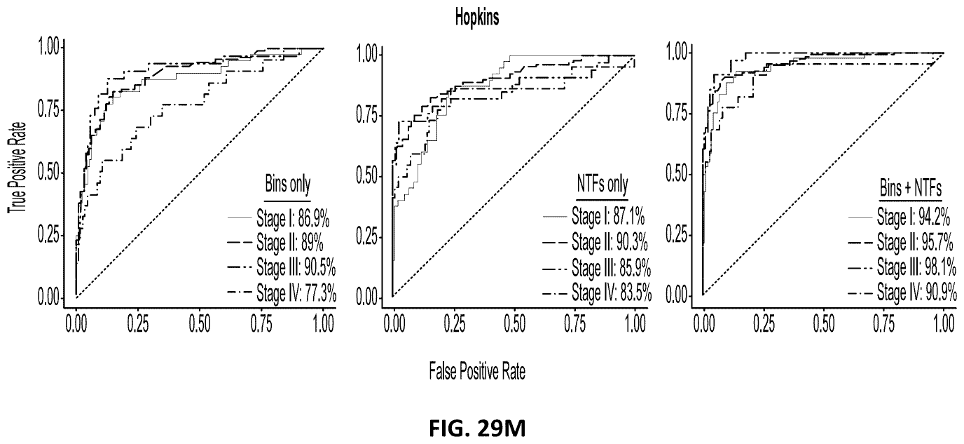

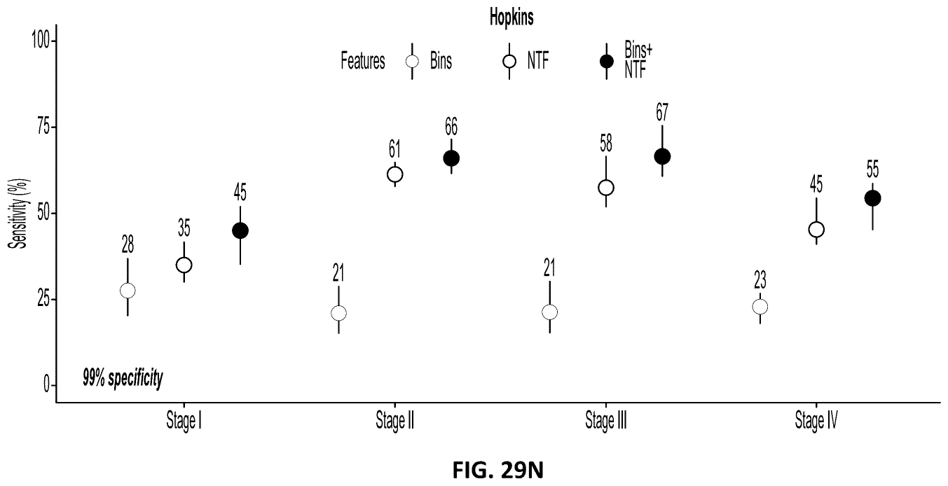

Human cell-free DNA (cfDNA) is commonly detected alongside metagenomic cell-free DNA, but their diagnostic compatibility for cancer detection remains unknown. Thus, two cfDNA cohorts (PMID: 25646427; 31142840, FIG. 29A) independent from the metagenomic assembly, on which we derived nine DNA fragment-end nucleotide frequencies (NTFs; PMID: 36630480) for synergy with metagenomic bins were explored.

HCC-bearing patients significantly differed by alpha and beta diversities (FIG. 29B). Subsequent LOOCV ML, matching CRC and lung-gut cohorts' methods, revealed strong bin-based discrimination (AUROC=91.74%; FIG. 29C, ‘Bins’), which the NTFs synergistically increased (AUROC=96.77%; FIG. 29C, ‘Bins+NTF’), increasing the bootstrapped median sensitivity at 99% specificity by ˜15% to 65.6% (FIG. 29D).

Whole genome sequenced (WGS) multi-cancer plasma samples (FIG. 29E) from Cristiano et al. (hereon “Hopkins”) were then analyzed, finding that cancer samples had significant differences in beta and alpha diversities (FIG. 29F). Per-cancer, 10-fold cross-validation (CV) using bin abundances versus healthy controls showed that the addition of NTFs improved performance in 6 of 7 cancer types and sensitivity in 5 of 7 cancer types (FIG. 29G-J). Aggregating all cancer versus non-cancer controls revealed significant improvement in multi-cancer detection with NTFs and bin abundances together for all stages, with an overall AUROC of 96.7% (Delong's: p≤4.11×10-8; FIG. 29K-N). Collectively, among at least 8 cancer types, the bins are diagnostically useful and broadly synergistic with human fragmentomic features.

### Evaluation of De Novo Assembled Metagenomic Bins in an Independent Cohort

Notwithstanding these findings, the diagnostic performance of the bins in a plasma dataset that (i) was independent from the metagenome assembly and (ii) utilized non-cancer controls were also evaluated. All 491 base-quality filtered, doubly host-depleted, length-restricted (>45 bp), shallow whole-genome sequenced (WGS) plasma samples from Cristiano were re-aligned. ML comparisons using raw metagenomic bin abundances for all cancer types versus 260 healthy controls, finding better-than-null performance for each of them (FIG. 9J), including lung cancer was then performed. Scrambled and shuffled negative control ML analyses showed expected null results (FIG. 9K). One-versus-all-others cancer type comparisons also confirmed better-than-null classification for 7 of 8 cancer types, with only bile duct cancer (n=25) failing to reach sufficient AUPR (FIG. 25F).

Calculation of robust Aitchison beta diversity distances additionally revealed clear separation along Axis 1 between healthy and pan-cancer samples (FIG. 9L; PERMANOVA: F=4.43, p=0.001). Subsequent ML comparisons between pan-cancer or grouped stages (FIGS. 9M-1 and 9M-2) intriguingly replicated a pattern originally observed in our CODICES cohort data (FIG. 27), wherein the metagenomic bins provided approximately similar diagnostic performance among clinical stages I-III samples (AUROC range: 86.35-92.86%) before showing the lowest performance on clinical stage IV samples (AUROC 95% CI: [76.16, 79.9]). The mechanism driving this pattern is unclear; it may artifactually stem from lower stage IV sample counts in both the Hopkins (n=22 samples) and CODICES cohorts, or potentially biologically denote non-cancer-associated microbial translocation due to gross vascular barrier defects. Regardless, all of these analyses strongly support the cancer type-specificity and diagnostic utility of these novel, metagenomic bins in several independent cohorts.

### Construction and Preliminary Evaluation of a Multi-Omic, Multi-Species Test for Lung Cancer Screening

To integrate heterogeneous multi-omic, multi-species data into a single test, a stacked ML strategy (FIG. 7A) was designed. It was intended to design a blood test that, if positive, that would trigger follow up confirmatory LDCT or PET-CT imaging (FIGS. 10A-1 and 10A-2, lower diagram “2”).

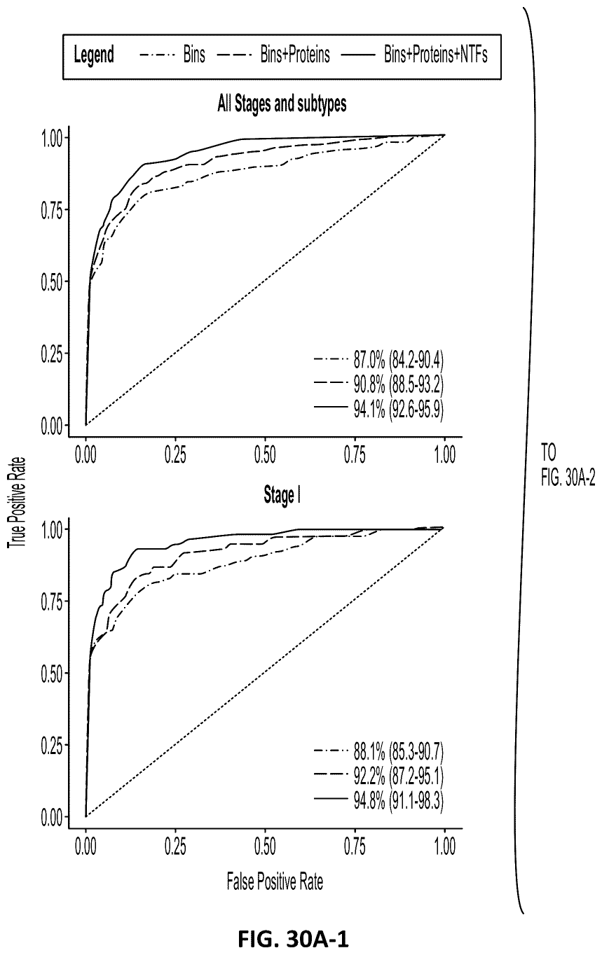

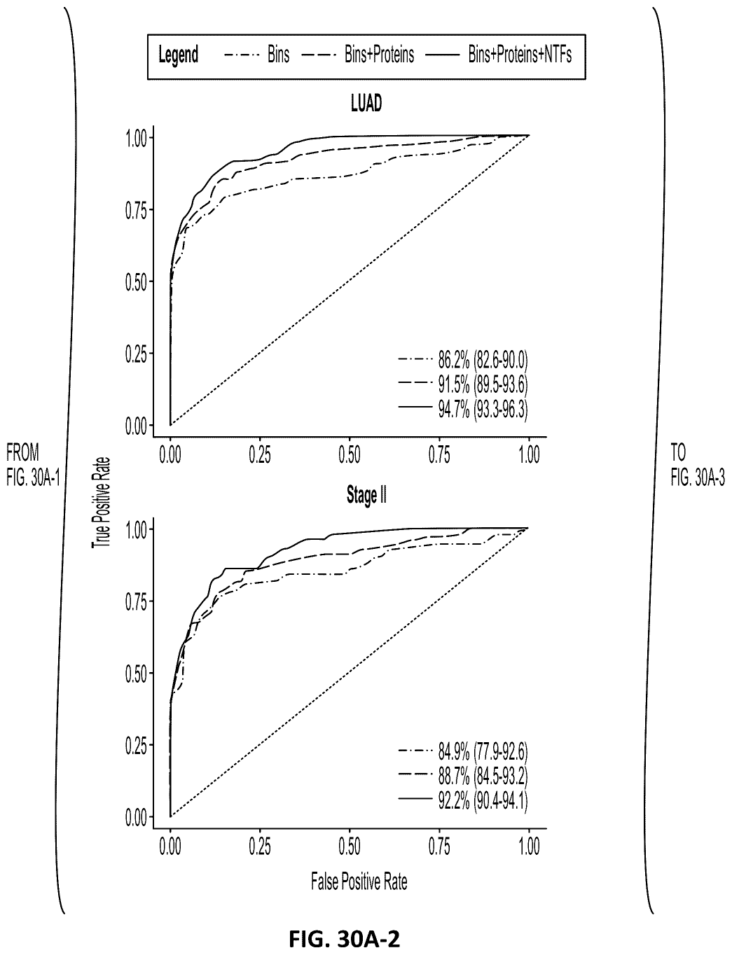

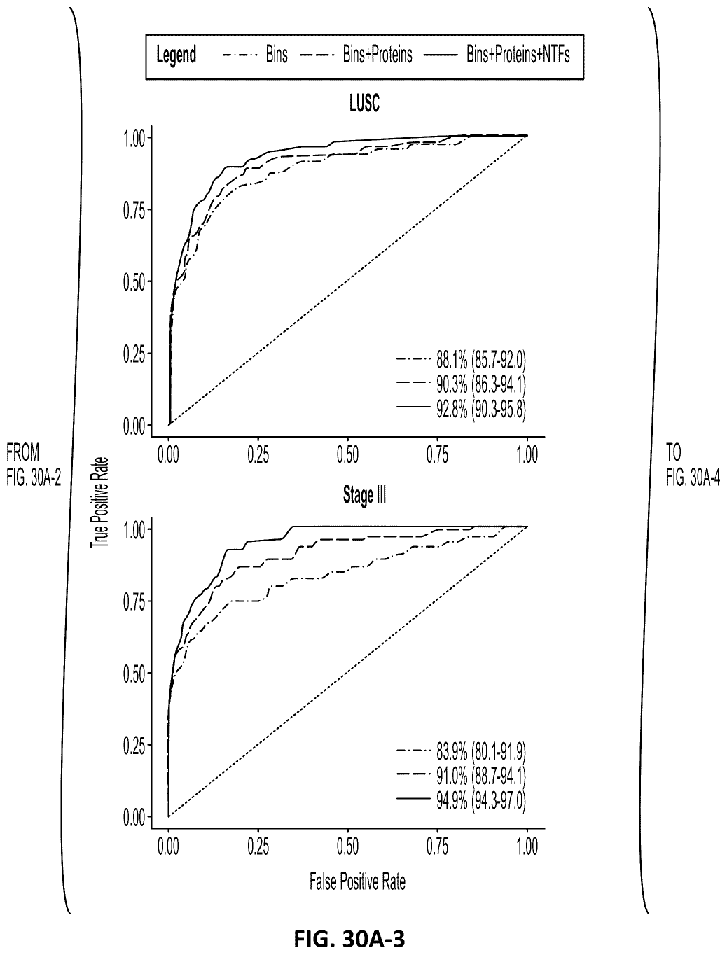

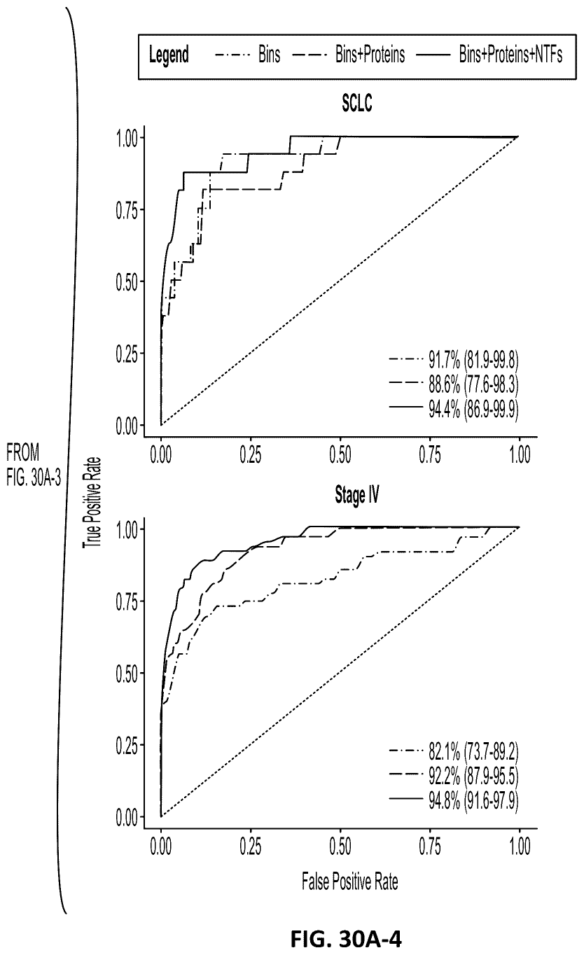

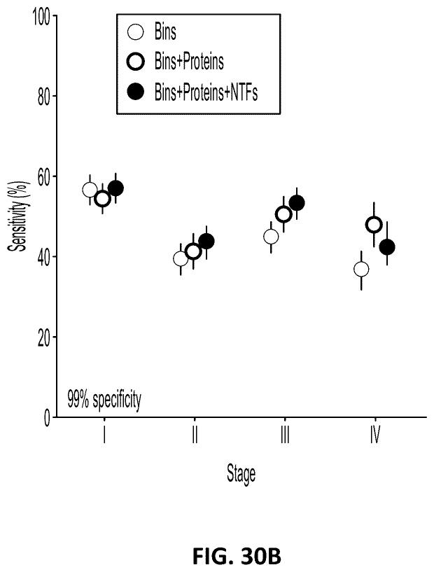

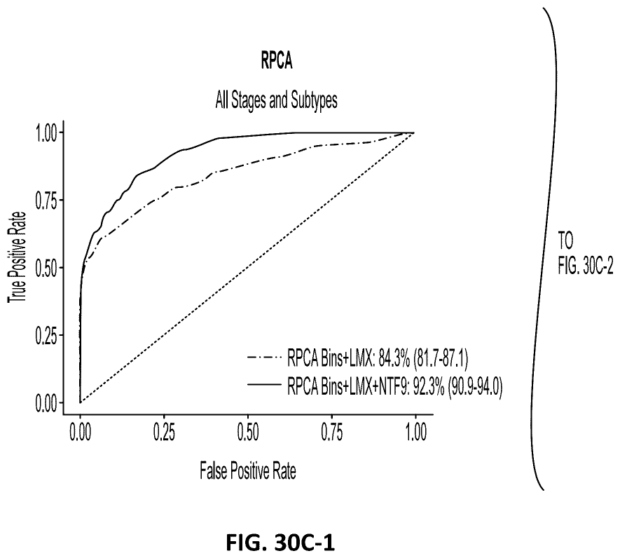

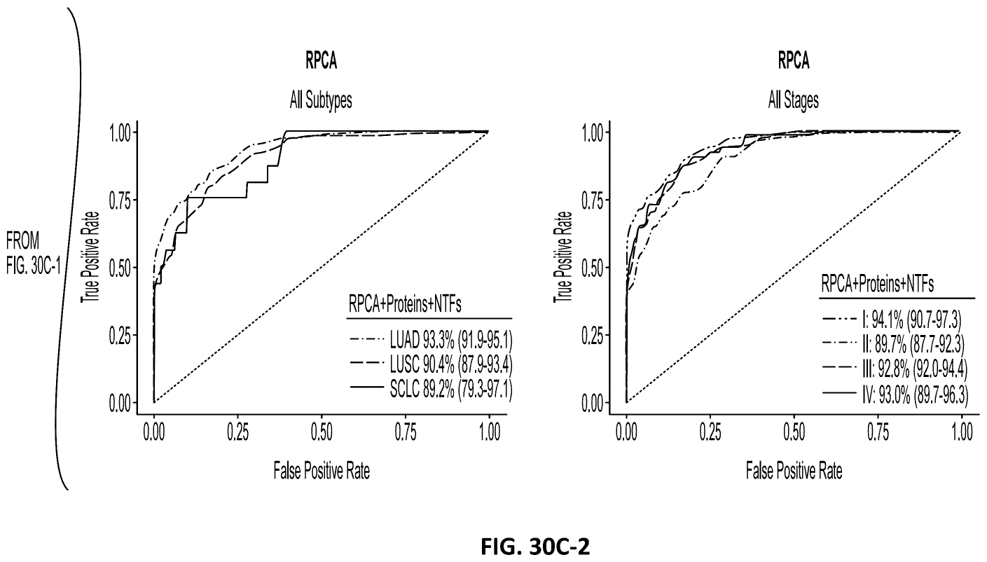

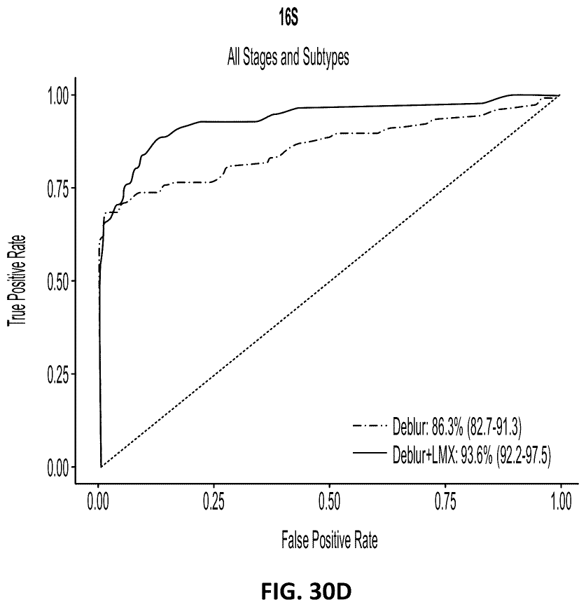

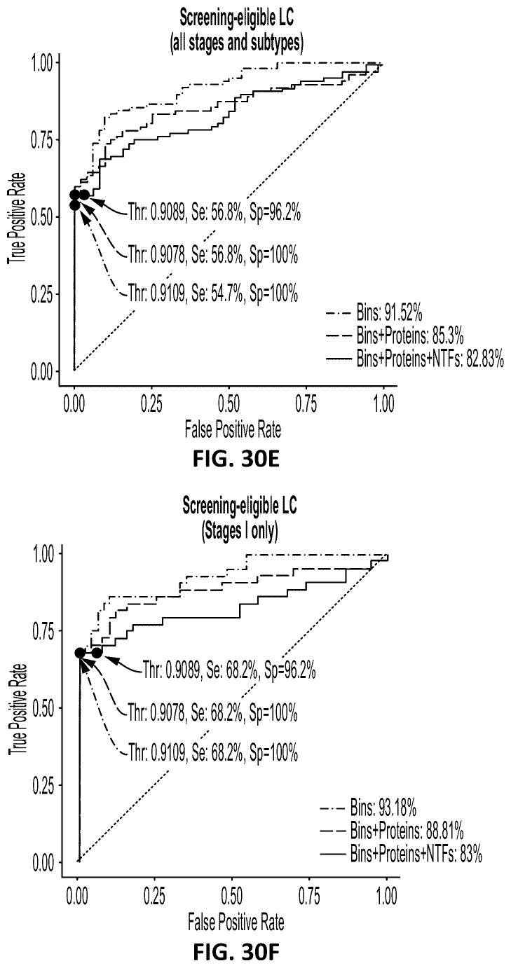

Stacked ML learning was applied using the bin abundances with or without human information (i.e., proteins and/or NTFs) to all LC and healthy samples (Overall AUROC=94.1%; FIGS. 30A-1 to 30A-4). The combined feature set significantly increased performance over any one individually (Delong's tests: q≤1.74×10-9). Bins and NTFs alone exhibited lower performance at stage IV, and 100 iterations of ML with stratified random sampling ruled out sample number bias; however, repeating this with combined feature sets provided the expected trend. Bootstrapping sensitivity at 99% specificity demonstrated stage I detection approaching 60% median sensitivity (FIG. 30B).

To ensure our cross-validated results were not influenced by overfitting, these analyses were repeated while using robust Aitchison PCA-projection (RPCA; PMID: 30801021) to transform bin abundances into just three principal component vectors, in combination with protein and NTF features (14 features total)—finding similar performance (AUROC=92.3%; FIGS. 30C-1 and 30C-2). Additionally, 142 LC and 96 healthy subjects processed in parallel for plasma-derived 16S rRNA amplicons (targeted microbial amplicon sequencing) independently replicated the results (AUROC=93.6%; FIG. 30D).

Since lung cancer screening by definition requires asymptomatic subjects with defined smoking histories and ages, all screening-eligible LC-bearing patients (n=95) and age-matched healthy smokers (n=46) (cases: 67.64±6.94 years; controls: 66.37±6.58 years) were subsetted as an internal validation cohort. After re-training stacked ML models on all other LC-bearing and healthy subjects, and deriving 99%-specificity-associated cutoffs, the re-trained stacked ML models were then applied on the held-out internal validation cohort (FIG. 30E). Although AUROCs decreased for the combined models, the specificities and sensitivities were similar (specificity: 96.2-100%, sensitivity: 54.7-56.8%); the bin-only model performed best (AUROC=91.52%)). Subsetting these predictions to stage I-only, screening-eligible LC patients while applying the same cutoffs revealed consistent 68.2% sensitivity at 96.2-100% observed specificity (FIG. 30F). Collectively, these data demonstrate that the combination of metagenomic (human, NTF, and microbial, bins) data with plasma proteins can yield a diagnostic classifier capable of discriminating subjects with lung cancer from healthy subjects.

### Development and Validation of a Clinico-Proteo-Metagenomic Diagnostic

Metagenomic bins significantly improved LD discrimination from LC over WIS-overlapping biomarkers, but did not alone, or with proteins, provide state-of-the-art diagnostic performance (FIGS. 8G-1 and 8G-2). The proteometagenomic classifier was then improved through the addition of clinically collected and radiographic-related metadata. Notably, the application of this diagnostic would occur after low-dose computed tomography (LDCT) in a high-risk clinical setting while maximizing test sensitivity to possibly rule out the need to biopsy a putatively non-malignant nodule (FIGS. 10A-1 and 10A-2, top); in contrast, the low-risk diagnostic described before, which relies only on metagenomic or proteometagenomic markers, would be applied to relatively healthy populations while maximizing specificity (FIGS. 10A-1 and 10A-2, bottom), as done by others to mitigate false positives. They represent two distinct approaches, both of which may be enhanced by metagenomic information.

More than 400 LC and LD samples in the CODICES cohort had matching clinical metadata (FIGS. 10B-1 and 10B-2), including most with lesion diameters, shapes (e.g., spiculation), solidity, location (e.g., upper lung), and nodule clinical risk scores such as Brock probability. Notably, the median lesion diameter in this cohort was just 2.5 centimeters (avg±SD=2.95±1.91 cm, n=431). Integrating these clinical variables with metagenomic bin abundances, as well as CEA and OPN concentrations, through stacked ML on all 454 samples substantially increased malignant nodule discrimination (AUROC=90.0%), such that it was greater than equivalent models built with matched clinical risk scores (Brock, pCA) or PET-CT-derived standard uptake values (SUVs) (AUROC range: 68.9-75.0%; FIG. 10C, left). Optionally adding PET-CT SUVs to this clinico-proteo-metagenomic diagnostic synergistically increased diagnostic performance (AUROC=91.8%). Remarkably, subsetting these predictions to lesions with known diameters ≤3 centimeters and low clinical risk scores of malignancy (pCA≤50%) led to increased performance for the diagnostic model but not for equivalent models built using Brock, pCA, or PET-CT SUVs (FIG. 10C, right).

The boost in proteometagenomic diagnostic performance through adding clinical metadata suggested that proteo-amplicon-based strategies may similarly benefit. We thus repeated the original LC (n=142) versus LD (n=97) proteo-amplicon analysis (FIGS. 26C-1 and 26C-2) with additional clinical information, finding synergistically improved performances for both taxonomic compositions (AUROC=91.8%) and functional pathway abundances (AUROC=91.3%) that outperformed clinical risk scores and PET-CT SUVs (FIG. 10D). Notably, the shape of these ROC curves (FIGS. 10C-10D) suggested that metagenomic features may aid a highly sensitive test (e.g., 97% sensitivity) while still maintaining sufficient specificity.

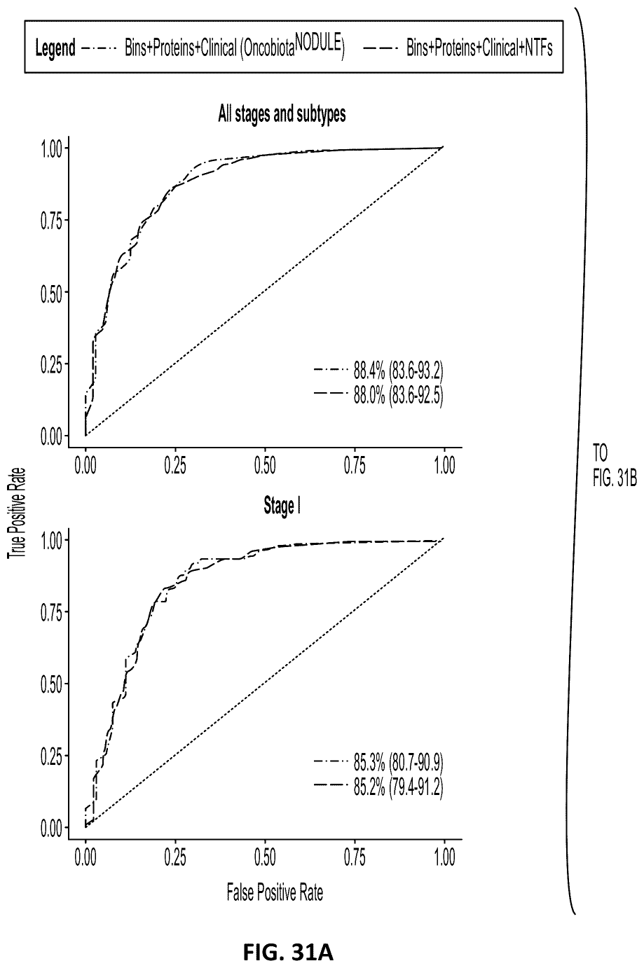

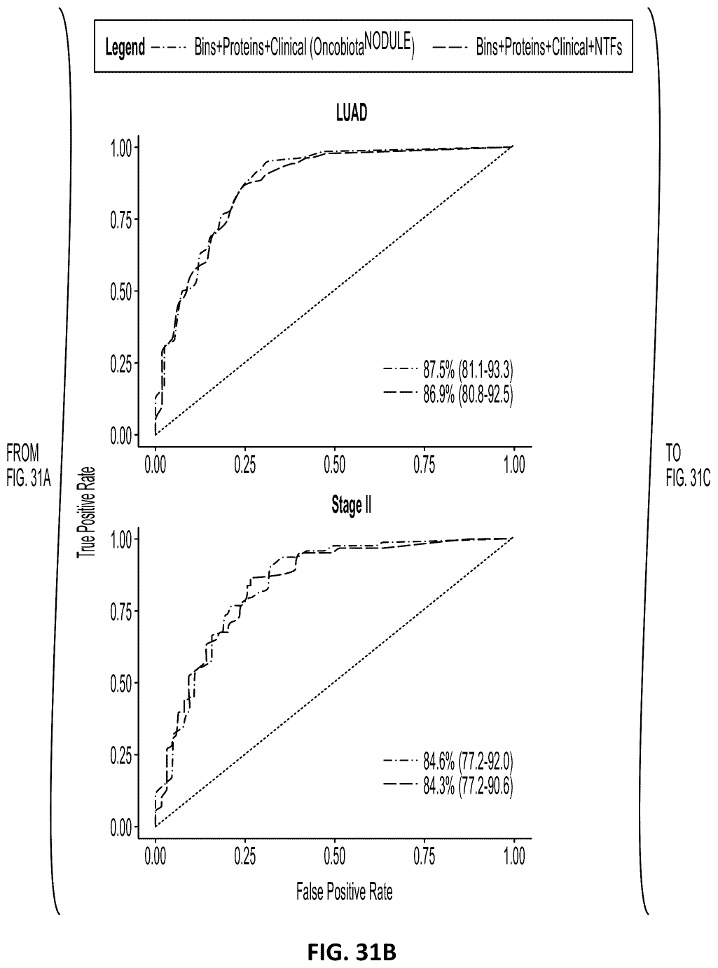

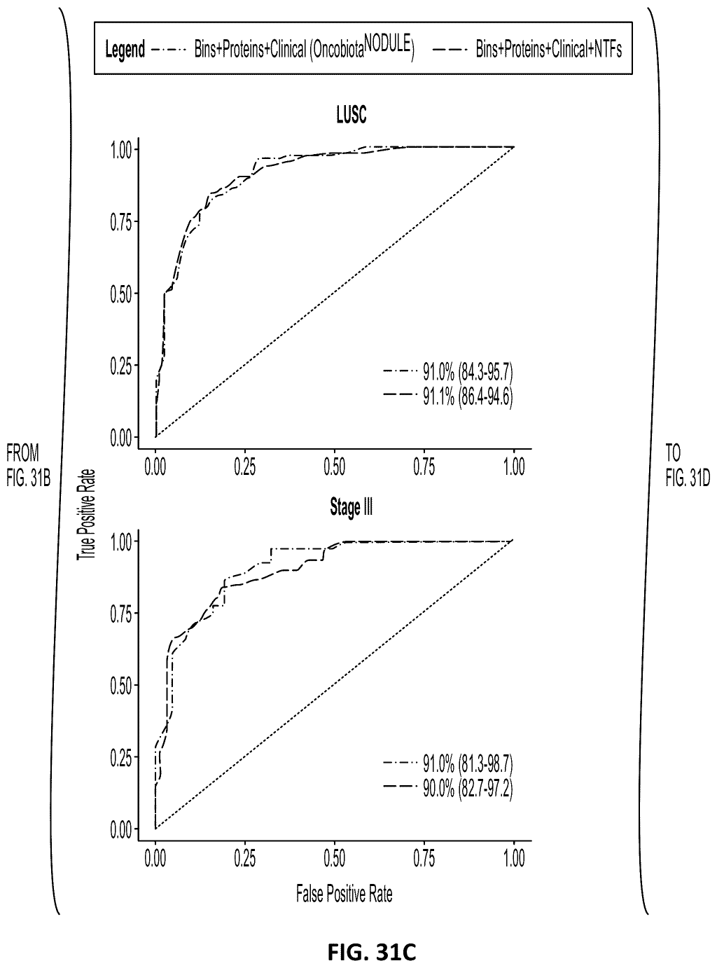

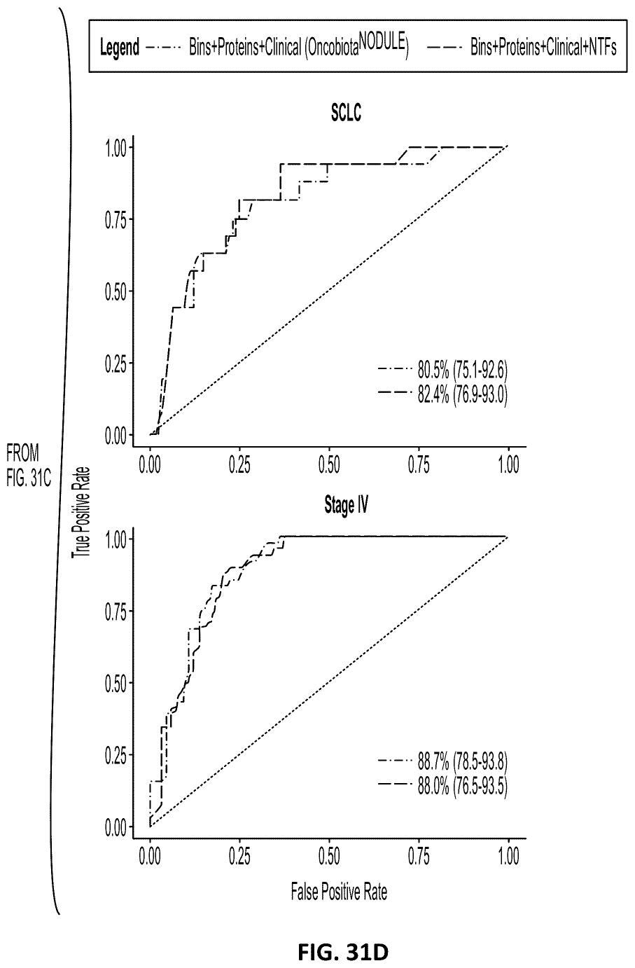

Encouraged by these improvements and intrigued by the cancer type specificity of metagenomic bins in Hopkins and TCGA cohorts, including between lung cancer histotypes, we hypothesized that the diagnostic model may be able to distinguish subtypes of lung cancer in sufficiently large cohorts. If realizable, blood-based discrimination between these two subtypes could valuably guide clinical management, since their histologies affect therapies differently (e.g., pemetrexed works in adenocarcinoma but not squamous counterparts). Since 293 (64.5%) of the 454 samples derived from either lung adenocarcinoma (LUAD) or lung squamous cell carcinoma (LUSC) (FIGS. 10B-1 and 10B-2), we constructed a LUAD versus LUSC classifier using the same clinico-proteo-metagenomic features as the diagnostic model (FIG. 10E). Notably, despite small nodules sizes (median=2.9 cm, mean=3.4 cm), the diagnostic model revealed strong discrimination (AUROC=91.5%), whereas equivalent models built using only PET-CT SUVs exhibited significantly worse performance (AUROC=68.6%), and pseudo-random performances when only using clinical risk scores or protein information (AUROC range: 47.9%-59.7%). Such findings suggest that plasma-centric metagenomic features may be able to guide minimally invasive histological subtype determination. Comparisons of lung cancer vs. lung disease classifier performance with and without NTF information revealed that, in this specific classification scenario, addition of NTFs did not lead to classifier improvements (FIGS. 31A-31D).

### Application of the Diagnostic Model on a Blinded Validation Cohort

Having thoroughly explored the CODICES discovery cohort, a final clinico-proteo-metagenomic diagnostic classifier using all 454 LC and LD samples across varying histotypes, and stages was then tuned. Other than metagenomic bins, CEA, and OPN, this classifier included, when available, clinical risk scores (Brock, pCA), lesion diameters, lesion spiculation, lesion solidity, upper lung nodule status, emphysema status, and smoking history. For comparability on later samples, equivalently stacked models using only clinical risk scores or PET-CT SUVs were also built.

A blinded validation cohort from collaborators comprising 106 plasma samples from patients, with an unknown number of clinical stage I lung cancer and lung diseases of diverse etiologies was then acquired. After extracting DNA from 400 μL of plasma for an independent sequencing run containing standard positive (mock community) and negative blank controls, and sequencing, non-human reads were aligned against the metagenomic bins. Metagenomic and proteomic features were then normalized for run-to-run variation, followed by feature standardization to match the diagnostic model inputs, including clinical metadata, when available. Notably, the average lesion diameter in this cohort was just 1.91 centimeters, with multiple lesions as small as 6 and 8 millimeters (FIG. 10F).

The final diagnostic model and an equivalent model using PET-CT information were then applied to the blinded cohort data, producing predictions in a one-way blinded manner. Remarkably, the diagnostic model significantly outperformed gold-standard PET-CT information (AUROC: 79.1% vs. 64.5%; Delong's test: p=0.019). Given the shape of the ROC curve from the 454 samples in the CODICES cohort (FIG. 10C), and in the blinded validation cohort (FIG. 10G), it was further hypothesized that the model test should provide significantly better specificity at a fixed 97% sensitivity. Indeed, this was the case when using comparative McNemar's tests between the model predictions fixed at 97% sensitivity and SUVs, as well as Brock and pCA (p<0.001 all tests), which additionally had inferior AUROCs (Brock: AUROC=0.724; pCA: AUROC=0.738). Additionally, sensitivity comparisons when fixed at 80% specificity were significantly better than PET-CT SUVs (p=0.00112) and appear to match or outperform fragmentomic approaches among stage I lung cancers. Thus, the model was validated in an independent, blinded validation cohort in the most challenging conditions of clinical stage I lung cancer versus lung disease while providing state-of-the-art diagnostic performance.

As described in Example 1, blood and tissue metagenomes in 17,520 samples from four independent cohorts across 34 cancer types, plus healthy controls and 11 diverse etiologies of lung diseases were characterized. Beginning with known cancer-associated bacterial and fungal biomarkers, it was demonstrated that as few as 300 plasma-derived microbial biomarkers can provide state-of-the-art lung cancer detection (˜50% sensitivity at 99% specificity) in the low-risk screening setting of newly collected 808 treatment-naive individuals across all stages and histological subtypes (FIGS. 7C-1 and 7C-2). It was further found that this parsimonious set of microbial biomarkers could be synergistically complemented by orthogonal, host-centric, plasma proteomic markers, CEA and OPN. Should performance outweigh parsimony, it was also found that BAL-associated metagenomic features provided up to ˜70% sensitivity at 99% specificity among clinical stage I disease (FIG. 7J). Notably, since these diagnostic models were constructed independently of ctDNA, fragmentomic, and epigenetic markers, the performances presented here likely represent the lower bound of metagenomic-augmented cancer diagnostic performances.

Notably, both WIS and BAL-associated biomarkers, which derived from intratissue or peri-tissue samples, respectively, were significantly enriched in microbes having ≥1% aggregate genomic coverage in the plasma-derived CODICES cohort (p<1×10-58 for both, Fisher's exact test). These data suggest that a substantial portion of the plasma metagenome is tumor tissue derived. The fact that diagnostic models improve when restricting to subjects with positive smoking histories also supports the hypothesis that chronic tissue damage may increase tissue-derived representation of concomitant metagenomes in plasma. This theory also has analogies in the gastrointestinal setting of colon cancer, in which degradation of the gut-vascular barrier enables bacterial translocation to the liver, which had implications for colorectal metastasis.

To address potential concerns around metagenomic read mis mapping of human DNA, the existence and utility of plasma-derived amplicon markers (FIG. 7K) were also evaluated. Indeed, either taxonomic compositions or inferred functional pathways from 16S rRNA data surprisingly showed similar diagnostic performance to shotgun methods, with equivalent performances when combined with CEA and OPN (FIG. 7K). Moreover, when shotgun-based diagnostic performances declined in LC versus LD comparisons, amplicon-based performance also declined (FIGS. 26C-1 and 26C-2), and both of them similarly increased in the context of additional clinico-proteomic features (FIG. 10D). Thus, plasma-derived, amplicon-based methods may provide a cost-, time-, and volume-efficient surrogate for shotgun-based metagenomic evaluation, warranting further investigation in lung cancer among others.

The observed decline in diagnostic performance between LCs and age-, sex-, and risk-matched, LD controls (FIGS. 8G-1 and 8G-2) additionally highlighted the importance of including these sample types in cell-free DNA screening studies to characterize true diagnostic performance in clinically realistic scenarios. This challenge also motivated metagenome (co-)assembly among 5187 whole-genome sequenced, cancer-associated, blood and tissue samples. By re-aligning identified metagenomic bins against 17,185 host-depleted samples, followed by comprehensive statistical and ML analyses, it was thoroughly demonstrated their cancer type specificity, diagnostic capacity, ability to mitigate batch effects in retrospectively collected data, and potential to non-invasively differentiate histological subtypes of lung cancer in small nodules. The broad generalizability of this metagenomic approach to other retrospectively collected cancer genomics cohorts may crucially help characterize functional repertoires of cancer-associated microbes.

Through the development of a multi-omic, multi-species (host and microbial), integrated diagnostic model, it was found that metagenomic-augmented approaches can provide superior sensitivity in early-stage disease than existing clinical risk scores and PET-CT imaging intensities (FIG. 10C). In particular, the validation and state-of-the-art performance of the diagnostic model on an exceptionally challenging, blinded validation cohort of clinical stage I cancers versus diverse lung diseases (FIGS. 10F-10H), underscores the broad utility that plasma metagenomes can have on future cancer diagnostics.

Our study has several caveats. Despite the large sample sizes of new and retrospective data, all plasma-derived microbial information is inherently low-abundance and difficult to sequence at high coverage. Attempts to calculate aggregated coverages can provide a proxy for which microbes may be present among sets of samples in our cohort, but they do not address microbial under sampling occurring in available data. Similarly, high aggregate coverages cannot distinguish between contaminants and non-contaminants.

Although multiple methods to control for possible contaminations were utilized, including numerous extraction blanks and positive controls on every sequencing run, it is not possible to rule out all false positive results. Nonetheless, independent replication of diagnostic performances with (i) separately processed, sequenced, and decontaminated amplicon-based approaches; (ii) independently-validated biomarker subsets (WIS and BAL); (iii) orthogonally-developed metagenomic bins; (iv) separate datasets, including those held out from the metagenome assemblies; and (v) a blinded validation cohort, all comprehensively suggest that the degree or rate of contamination does not preclude generalizable, clinically useful conclusions.

This comprehensive analysis of plasma metagenomes for the early-stage detection of lung cancer provides a generalizable approach for developing or augmenting cancer diagnostics with metagenomic information to benefit patients worldwide.

What is claimed is:

1. A method of determining a disease of a subject, comprising:
(a) receiving a biological sample, electronic medical record information, and radiologic data of said subject;
(b) sequencing a plurality of non-human nucleic acid molecules of said biological sample thereby generating a plurality of microbial sequencing reads; and
(c) processing said plurality of microbial sequencing reads, electronic medical record information, and radiologic data with a set of trained predictive models, thereby determining said disease of said subject with at least about 80% accuracy,
wherein said set of trained predictive models comprises a first predictive model and a second predictive model, wherein said first predictive model is trained with a plurality of microbial abundances and corresponding health states, and
wherein said second predictive model processes an output of said first predictive model.

2. The method of claim 1, wherein said first predictive model comprises a machine learning, neural network, a naïve Bayes, convolutional neural network, random forest, support vector machines, or any combination thereof models, and wherein said second predictive model comprises a logistic regression model.

3. The method of claim 1, wherein said sample comprises a liquid biopsy, and wherein said liquid biopsy comprises plasma, serum, whole blood, urine, stool, cerebral spinal fluid, saliva, sweat, tears, exhaled breath condensate, or any combination thereof.

4. The method of claim 1, wherein said sequencing comprises shotgun sequencing.

5. The method of claim 1, comprising receiving a concentration of one or more plasma proteins.

6. The method of claim 1, comprising aligning a plurality of nucleic acid molecule sequencing reads of said biological sample with a human reference genome to identify a plurality of non-human nucleic acid molecule sequencing reads.

7. The method of claim 6, further comprising aligning said plurality of non-human nucleic acid molecule sequencing reads to a database of microbial genomes to identify said plurality of microbial sequencing reads.

8. The method of claim 7, wherein said database comprises a de novo metagenomic assembly comprising genomic contigs.

9. The method of claim 8, wherein said genomic contigs comprise one or more metagenomic bins.

10. The method of claim 9, wherein aligning said plurality of non-human nucleic acid molecule sequencing reads to said de novo metagenomic assembly produces an aligned bin abundances of said plurality of non-human nucleic acid molecule sequencing reads.

11. The method of claim 10, wherein said trained predictive model is configured to process said aligned bin abundances of said subject's plurality of non-human nucleic acid molecule sequencing reads.

12. The method of claim 1, comprising determining one or more features of said radiologic data, wherein said one or more features of said radiologic data are processed by said trained predictive model, and wherein said one or more features of said radiologic data comprise Brock cancer probability score, Mayo clinic risk score for nodule malignancy, cancer lesion diameter, cancer lesion spiculation, cancer lesion solidity, or any combination thereof.

13. The method of claim 1, wherein said disease comprises lung cancer, and wherein said lung cancer comprises a tumor mass with a diameter less than about 3 centimeters or less than about 8 millimeters.

14. The method of claim 13, wherein said trained predictive model is configured to determine a stage of said lung cancer, anatomical origin of said lung cancer, or a combination thereof.

15. The method of claim 1, wherein said health state comprises cancer, non-cancerous disease, or healthy.

16. A system configured to determine a disease of a subject, comprising:
(a) one or more processors; and
(b) a non-transient computer readable storage medium including software, wherein the software comprises executable instructions that, as a result of execution, cause the one or more processors of the computer system to:
(i) receive a plurality of non-human nucleic acid molecule sequencing reads of a biological sample, electronic medical record information, and radiologic data of said subject; and
(ii) process said plurality of microbial nucleic acid molecule sequencing reads, electronic medical record information, and radiologic data of said subject with a set of trained predictive models, thereby determining said disease of said subject with at least about 80% accuracy,
wherein said set of trained predictive models comprises a first predictive model and a second predictive model, wherein said first predictive model is trained with a plurality of microbial abundances and corresponding health states, and
wherein said second predictive model processes an output of said first predictive model.

17. The system of claim 16, wherein said first predictive model comprises a machine learning, neural network, a naïve Bayes, convolutional neural network, random forest, support vector machines, or any combination thereof models, and wherein said second predictive model comprises a logistic regression model.

18. The system of claim 16, wherein said sample comprises a liquid biopsy, and wherein said liquid biopsy comprises plasma, serum, whole blood, urine, stool, cerebral spinal fluid, saliva, sweat, tears, exhaled breath condensate, or any combination thereof.

19. The system of claim 16, wherein said plurality of microbial nucleic acid molecule sequencing reads are generated by shotgun sequencing.

20. The system of claim 16, wherein said executable instructions comprises receiving a concentration of one or more plasma proteins.

21. The system of claim 16, wherein said executable instructions cause said one or more processors to align a plurality of nucleic acid molecule sequencing reads of said biological sample with a human reference genome library to identify said plurality of microbial nucleic acid molecule sequencing reads.

22. The system of claim 16, wherein said executable instructions cause said one or more processors to align a plurality of non-human nucleic acid molecule sequencing reads of said plurality of non-human nucleic acid molecules to a database of microbial genomes to identify said plurality of microbial sequencing reads.

23. The system of claim 22, wherein said database comprises a de novo metagenomic assembly comprising genomic contigs.

24. The system of claim 23, wherein said genomic contigs comprise one or more metagenomic bins.

25. The system of claim 23, wherein aligning said plurality of non-human nucleic acid molecule sequencing reads to said de novo metagenomic assembly produces an aligned bin abundances of said plurality of non-human nucleic acid molecule sequencing reads.

26. The system of claim 25, wherein said trained predictive model is configured to process said aligned bin abundances of said subject's plurality of non-human nucleic acid molecule sequencing reads.

27. The system of claim 16, wherein said executable instructions cause said one or more processors to determine one or more features of said radiologic data, wherein said one or more features of said radiologic data are processed by said trained predictive model, and wherein said one or more features of said radiologic data comprise Brock cancer probability score, Mayo clinic risk score for nodule malignancy, cancer lesion diameter, cancer lesion spiculation, cancer lesion solidity, or any combination thereof.

28. The system of claim 16, wherein said disease comprises lung cancer, and wherein said lung cancer comprises a tumor mass with a diameter up to about 3 centimeters or up to about 8 millimeters.

29. The system of claim 28, wherein said trained predictive model is configured to determine a stage of said lung cancer, anatomical origin of said lung cancer, or a combination thereof.

30. The system of claim 16, wherein said health state comprises cancer, non-cancerous disease, or healthy.
